(19) United States
(12) Patent Application Publication
(10) Pub. No.: US 2026/0199464 A1
(43) Pub. Date: Jul. 16, 2026

(54) COMPOSITIONS AND METHODS FOR ENHANCING CAR-T CELL PERFORMANCE AND FOR CANCER IMMUNOTHERAPY

(71) Applicant: The Regents of the University of California, Oakland, CA (US)

(72) Inventors: Ananda GOLDRATH, San Diego, CA (US); Miguel REINA-CAMPOS, San Diego, CA (US); Giovanni GALLETTI, San Diego, CA (US)

(21) Appl. No.: 19/130,906

(22) PCT Filed: Nov. 17, 2023
(86) PCT No.: PCT/US2023/080254

§ 371 Date: May 18, 2025

Related U.S. Application Data
(60) Provisional application No. 63/426,327, filed on Nov. 17, 2022.
(60) Provisional application No. 63/527,989, filed on Jul. 20, 2023.

(51) Int. Cl. A61K 40/11; A61K 31/353; A61K 31/357; A61K 31/366; A61K 31/553; A61K 35/17; A61K 38/00; A61K 40/31; A61P 35/00; C07K 16/28; C07K 16/40; C12N 5/0783; C12N 5/10; C12N 9/10

(52) U.S. Cl. CPC A61K 40/11; A61K 31/353; A61K 31/357; A61K 31/366; A61K 31/553; A61K 35/17; A61K 38/00; A61K 40/31; A61P 35/00; C07K 16/2818; C07K 16/40; C12N 5/0636; C12N 5/10; C12N 9/1085; C12Y 205/01021; C12N 2510/00

(57) ABSTRACT

Provided are methods for metabolically enhancing CAR-T cell function in vitro and in vivo, and their persistence in vivo, wherein the enhancing of CAR-T cells can be used for the enhancement of CAR-T cell efficacy in cancer immunotherapy. Provided herein are methods for enhancing the ability of chimeric antigen receptor (CAR)-CD8 T cells to persist longer in solid tumors by inhibiting a squalene synthase (or Fdft1), an enzyme of the mevalonate/cholesterol synthesis pathway. In alternative embodiments, provided herein are CAR-T cells that have been treated in vivo or ex vivo with an agent, to delete or diminish the activity of a squalene synthase in the CAR-T cell. Provided herein are methods for treating a cancer comprising administration of an agent, which for example can be an inhibitory nucleic acid or a small molecule inhibitor, to delete or diminish the activity of a squalene synthase in the cancer cell.

### RELATED APPLICATIONS

This Patent Convention Treaty (PCT) International Application claims the benefit of priority under 35 U.S.C. § 119(e) of U.S. Provisional Patent Application Ser. No. 63/426,327, filed Nov. 17, 2022; and U.S. Ser. No. 63/527,989, filed Jul. 20, 2023. The aforementioned applications are expressly incorporated herein by reference in their entirety and for all purposes. All publications, patents, patent applications cited herein are hereby expressly incorporated by reference for all purposes.

### STATEMENT AS TO FEDERALLY SPONSORED RESEARCH

This invention was made with government support under AI067545, AI150282, AI072117, and AI132122, awarded by the National Institutes of Health. The government has certain rights in the invention.

### TECHNICAL FIELD

This invention generally relates to cell biology and cancer therapies. In alternative embodiments, provided are methods for metabolically enhancing chimeric antigen receptor (CAR)-T cell function and persistence, wherein the enhancing of CAR-T cells or genetically engineered T cells can be used for the enhancement of CAR-T cell or genetically engineered T cell efficacy in cancer immunotherapy. In alternative embodiments, provided are products of manufacture and kits comprising CAR-T cells or genetically engineered T cells genetically altered to have less than wild type or substantially no expression or activity of a squalene synthase (or Fdft1), an enzyme of the mevalonate/cholesterol synthesis pathway. In alternative embodiments, provided are methods for metabolically enhancing chimeric antigen receptor (CAR)-T cell function and persistence comprising administering a CAR-T cell with an inhibitor of a squalene synthase (or Fdft1). In alternative embodiments, provided herein are CAR-T cells or genetically engineered T cells that have been treated in vivo or ex vivo (before being administered in vivo) with an agent, which for example can be an inhibitory nucleic acid or a small molecule inhibitor, to delete or diminish the activity of a squalene synthase (or Fdft1) in the CAR-T cell or genetically engineered T cell. In alternative embodiments, provided herein are methods for treating a cancer comprising administration of an agent, which for example can be an inhibitory nucleic acid or a small molecule inhibitor, to delete or diminish the activity of a squalene synthase (or Fdft1) in the cancer cell. In alternative embodiments, provided herein are methods for treating a cancer comprising administration of a squalene synthase (or Fdft1) together, or in conjunction with, a PK1 inhibitor.

### BACKGROUND

Chimeric antigen receptor (CAR) (CARs are also known as chimeric immunoreceptors, chimeric T cell receptors or artificial T cell receptors) T cell (CAR-T cell) therapies can be very effective in treating cancers and other diseases by their ability to target a specific antigen, for example, a cancer antigen.

CARs are receptor proteins that have been engineered to give T cells (particularly T cells, a type of lymphocyte, that can kill a cancer cell when the T cell specifically binds to that cancer cell, such as for example, a CD8+ T cell) a new ability to target a specific antigen such as a cancer antigen (a molecule just found on a cancer cell). The CAR receptors are chimeric because they combine both antigen-binding and T cell activating (for example, cancer killing) functions into a single receptor. Thus, after CAR T cells are infused into a patient, they act as a “living drug” against cancer cells. When the CAR-T cells come in contact with their targeted antigen on a cancer cell, CAR T cells bind to the cancer cell and become activated, then proceed to proliferate and become cytotoxic to the cancer cell.

Tissue-resident memory CD8 T cells (T_{RM}) offer rapid and long-term protection at sites of re-infection¹. Tumor-infiltrating lymphocytes (TIL) with characteristics of T_{RM} maintain enhanced effector functions, predict responses to immunotherapy, and accompany better prognoses^{2,3}. Thus, an improved understanding of the metabolic strategies that enable tissue residency by T cells could inform new approaches to empower immune responses in tissues and solid tumors.

### SUMMARY

In alternative embodiments, provided are methods for metabolically enhancing chimeric antigen receptor (CAR)-T cells, including genetically engineered CD8 T cells which target cancer cells, thereby enhancing cell function and persistence, wherein the enhancing of CAR-T cells or genetically engineered T cells and genetically engineered CD8 T cells can be used for the enhancement of CAR-T cell efficacy in cancer immunotherapy.

In alternative embodiments, provided are CAR-T cells or genetically engineered CD8 T cells or a pharmaceutical formulation comprising a CAR-T cell or genetically engineered CD8 T cells, wherein the CAR-T cell or genetically engineered CD8 T cell is genetically modified to have no expression of, or substantially no expression or activity of, or less than wild type levels of expression or activity of, a squalene synthase or Fdft1 enzyme or gene; and optionally the CAR-T cell or genetically engineered CD8 T cell is formulated as a pharmaceutical, or formulated for in vivo administration.

In alternative embodiments, provided are CAR-T cells or genetically engineered CD8 T cells or a pharmaceutical formulation comprising a CAR-T cell or genetically engineered CD8 T cell, wherein the CAR-T cell or genetically engineered CD8 T cell comprises or has contained therein a compound or composition capable of generating a phenotype of: no expression of, or substantially no expression or activity of, or less than wild type levels of expression or activity of, a squalene synthase or Fdft1 enzyme or gene; and optionally the CAR-T cell or genetically engineered CD8 T cell is formulated or manufactured as or with a pharmaceutical, an implant, a device or is formulated for in vivo administration.

In alternative embodiments, provided are products of manufacture or kit, or a pharmaceutical formulation, comprising a CAR-T cell or genetically engineered CD8 T cell as provided herein.

In alternative embodiments, provided are methods for:
metabolically enhancing chimeric antigen receptor (CAR)-T cell or genetically engineered CD8 T cell function and persistence in vitro or in vivo, or
enhancing CAR-T cell or genetically engineered CD8 T cell efficacy in cancer immunotherapy,
the method comprising:
(a) administering to an individual in need thereof a CAR-T cell or genetically engineered CD8 T cell with an inhibitor of a squalene synthase or Fdft1,
wherein optionally the inhibitor of a squalene synthase or Fdft1 is a small molecule, a peptide or a polypeptide, or a nucleic acid inhibitor of squalene synthase or Fdft1 expression or activity,
and optionally the CAR-T cell or genetically engineered CD8 T cell is contacted with the squalene synthase or Fdft1 inhibitor before in vivo administration to an individual in need thereof, during or with in vivo administration of the CAR-T cell or genetically engineered CD8 T cell, and/or after in vivo administration of the CAR-T cell or genetically engineered CD8 T cell; and/or
(b) administering to an individual in need thereof a CAR-T cell or genetically engineered CD8 T cell that has been ex vivo exposed to an inhibitor of a squalene synthase or Fdft1,
wherein optionally the inhibitor of a squalene synthase or Fdft1 is a small molecule, a peptide or a polypeptide or a nucleic acid inhibitor of squalene synthase or Fdft1 expression or activity,
and optionally the nucleic acid inhibitor of squalene synthase or Fdft1 expression or activity comprises an antisense or miRNA molecule,
and optionally the polypeptide inhibitor of squalene synthase or Fdft1 expression or activity comprises an anti-squalene synthase or Fdft1 antibody; and/or
(c) (b) administering to an individual in need thereof a CAR-T cell or genetically engineered CD8 T cell that has been genetically modified to have a phenotype comprising: no expression of, or substantially no expression or activity of, or less than wild type levels of expression or activity of, a squalene synthase or Fdft1 enzyme or gene.

In alternative embodiments, provided are uses of a product of manufacture or kit, or a pharmaceutical formulation, comprising a CAR-T cell or genetically engineered CD8 T cell as provided herein, to:
metabolically enhancing chimeric antigen receptor (CAR)-T cell or genetically engineered CD8 T cell function and persistence in vitro or in vivo, or
enhancing CAR-T cell or genetically engineered CD8 T cell efficacy in cancer immunotherapy.

In alternative embodiments, provided are products of manufacture or kit, or a pharmaceutical formulation as provided herein, for use in:
metabolically enhancing chimeric antigen receptor (CAR)-T cell or genetically engineered CD8 T cell function and persistence in vitro or in vivo, or
enhancing CAR-T cell efficacy or genetically engineered CD8 T cell in cancer immunotherapy.

In alternative embodiments, the small molecule Fdft1 inhibitor comprises:
(a) mevastatin, or a compound having the formula:

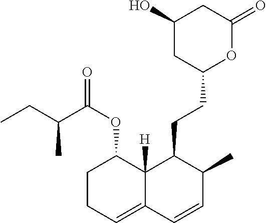 [SMILES: CC[C@H](C)C(=O)O[C@H]1CCC=C2C=C[C@H](C)[C@H](CC[C@@H]3C[C@@H](O)CC(=O)O3)[C@H]21]

(b) zaragozic acid A (ZAA), or a compound having the formula:

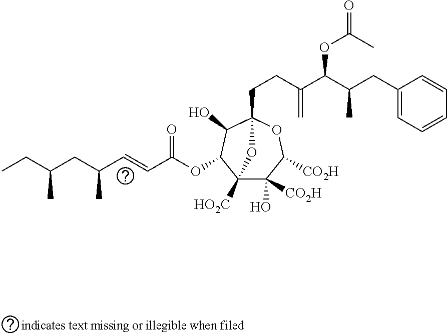

(c) a compound having the formula:

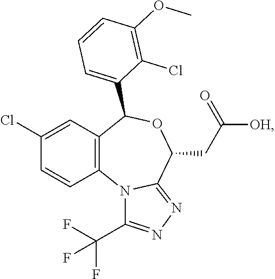 [SMILES: COc1cccc([C@H]2O[C@H](CC(=O)O)c3nnc(C(F)(F)F)n3-c3ccc(Cl)cc32)c1Cl]

(d) bavachinin, or a compound having the formula:

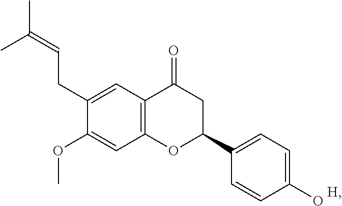 [SMILES: COc1cc2c(cc1CC=C(C)C)C(=O)C[C@@H](c1ccc(O)cc1)O2]

or
(e) a salt, an isomer, deuterated isoform, optical isomer or stereoisomer, a racemate or racemic mixture, an enantiomer, an individual diastereomer or a diastereomeric mixture, an analog, a crystalline product or a crystalline intermediate, a pharmaceutically acceptable salt thereof, a prodrug or a bioisostere of any of (a) to (d).

In alternative embodiments, provided are methods for treating or ameliorating a cancer in an individual in need thereof comprising administering to the individual in need thereof an inhibitor of a squalene synthase or an Fdft1 inhibitor,
wherein optionally the inhibitor of a squalene synthase or Fdft1 is a small molecule, a peptide or a polypeptide, or a nucleic acid inhibitor of squalene synthase or Fdft1 expression or activity,
and optionally the nucleic acid inhibitor of squalene synthase or Fdft1 inhibitor expression or activity comprises an antisense or miRNA molecule, or a CRISPR cassette or nucleic acid module,
and optionally the polypeptide inhibitor of squalene synthase or Fdft1 inhibitor expression or activity comprises an anti-squalene synthase or Fdft1 antibody.

In alternative embodiments of methods for treating or ameliorating a cancer:
the methods further comprise administration, or co-administration, of a programmed cell death protein 1 (PD-1) inhibitor, wherein optionally the PD1 inhibitor is pembrolizumab (or KEYTRUDA™), nivolumab (or OPDIVO™) or cemiplimab (or LIBTAYO™);
the small molecule Fdft1 inhibitor comprises:
(a) mevastatin, or a compound having the formula:

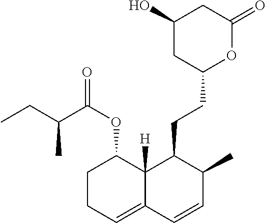 [SMILES: CC[C@H](C)C(=O)O[C@H]1CCC=C2C=C[C@H](C)[C@H](CC[C@@H]3C[C@@H](O)CC(=O)O3)[C@H]21]

(b) zaragozic acid A (ZAA), or a compound having the formula:

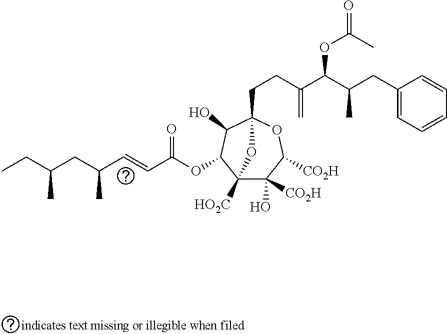

(c) a compound having the formula:

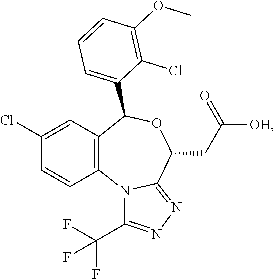 [SMILES: COc1cccc([C@H]2O[C@H](CC(=O)O)c3nnc(C(F)(F)F)n3-c3ccc(Cl)cc32)c1Cl]

(d) bavachinin, or a compound having the formula:

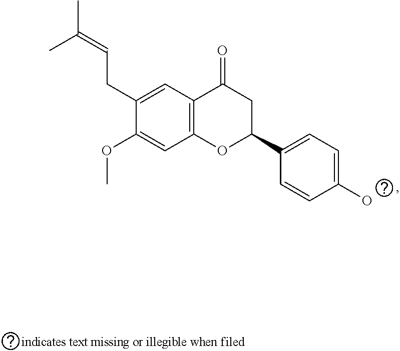 [SMILES: COc1cc2c(cc1CC=C(C)C)C(=O)C[C@@H](c1ccc(O)cc1)O2]

or
(e) as salt, an isomer, deuterated isoform, optical isomer or stereoisomer, a racemate or racemic mixture, an enantiomer, an individual diastereomer or a diastereomeric mixture, an analog, a crystalline product or a crystalline intermediate, a pharmaceutically acceptable salt thereof, a prodrug or a bioisostere of any of (a) to (d).

In alternative embodiments, provided are inhibitors of a squalene synthase or an Fdft1 inhibitor for use in treating cancer,
wherein optionally the inhibitor of a squalene synthase or Fdft1 is a small molecule, a peptide or a polypeptide, or a nucleic acid inhibitor of squalene synthase or Fdft1 expression or activity,
and optionally the nucleic acid inhibitor of squalene synthase or Fdft1 inhibitor expression or activity comprises an antisense or miRNA molecule, or a CRISPR cassette or nucleic acid module,
and optionally the polypeptide inhibitor of squalene synthase or Fdft1 inhibitor expression or activity comprises an anti-squalene synthase or Fdft1 antibody,
and optionally the inhibitor of a squalene synthase or the Fdft1 inhibitor is administered with, or formulated with, a programmed cell death protein 1 (PD-1) inhibitor.

In alternative embodiments, provided are uses of an inhibitor of a squalene synthase or an Fdft1 inhibitor for the manufacture of a medicament for treating cancer wherein optionally the inhibitor of a squalene synthase or Fdft1 is a small molecule, a peptide or a polypeptide, or a nucleic acid inhibitor of squalene synthase or Fdft1 expression or activity,
and optionally the nucleic acid inhibitor of squalene synthase or Fdft1 inhibitor expression or activity comprises an antisense or miRNA molecule, or a CRISPR cassette or nucleic acid module,
and optionally the polypeptide inhibitor of squalene synthase or Fdft1 inhibitor expression or activity comprises an anti-squalene synthase or Fdft1 antibody,
and optionally the inhibitor of a squalene synthase or the Fdft1 inhibitor is administered with, or formulated with, a programmed cell death protein 1 (PD-1) inhibitor.

In alternative embodiments of methods and/or uses as provided herein, for example, for treating or ameliorating a cancer, the cancer is melanoma, advanced melanoma, cutaneous or intraocular melanoma, primary neuroendocrine carcinoma of the skin, breast cancer, a cancer of the head and neck, uterine cancer, rectal and colorectal cancer, a cancer of the head and neck, cancer of the small intestine, a colon cancer, a cancer of the anal region, a stomach cancer, lung cancer, brain cancer, non-small-cell lung cancer, ovarian cancer, angiosarcoma, bone cancer, osteosarcoma, prostate cancer; cancer of the bladder; cancer of the kidney or ureter or renal cell carcinoma, or carcinoma of the renal pelvis; a neoplasm of the central nervous system (CNS) or renal cell carcinoma.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1, FIG. 2 and FIG. 3 schematically illustrate exemplary actionable metabolic interventions to enhance antitumor CD8 T Cell function.

FIG. 4A and FIG. 4B graphically illustrate data showing that loss of Fdft1 promotes TIL persistence and increased antitumor function.

FIG. 5 graphically illustrates data showing an increase in upstream derivates from FPP metabolism (Farnesols): dolichol; ubiquinone; geranylgeranylation; and, isoprenoid chains.

FIG. 6 graphically illustrates data showing that antitumor actions of zaragozic acid A (ZAA) depend on the immune system.

FIG. 7 graphically illustrates data showing that tumor Growth Curves in B16-F10 tumors implanted subcutaneously (s.c.) in B6 mice.

FIG. 8 graphically illustrates data showing that ZAA synergizes with anti-PD1 therapy.

FIG. 9 graphically illustrates data showing that ZAA synergizes with CD45 therapy.

FIG. 10A-I illustrate functional genetics in vivo, metabolomics and transcriptional analysis of ex vivo populations of memory CD8 T cells identify a graded upregulation of a Srebp2-dependent metabolic programming across T_{RM}: FIG. 10A schematically illustrates experimental design and data analysis approach for an in vivo CRISPR/Cas9-mediated loss-of-function screen of metabolic regulators of memory CD8 T cell differentiation in LCMV infection;

FIG. 10B illustrates an unsupervised hierarchical clustering heatmap of averaged gene essentiality scores for significant genes in SI T_{RM} in both libraries;

FIG. 10C illustrates an unsupervised hierarchical clustering of scaled averaged enrichment scores of significant metabolic signatures in SI T_{RM} in both libraries;

FIG. 10D graphically illustrates data showing the PCA of relative metabolite abundances of indicated P14 CD8 T cells 13 days after LCMV infection;

FIG. 10E graphically illustrates data showing the abundance of selected identified annotated metabolites of mevalonate/cholesterol synthesis pathway on indicated P14 CD8 T cells;

FIG. 10F graphically illustrates data showing mevalonate/cholesterol synthesis pathway GSVA scores from RNAseq analysis of indicated P14 CD8 T cells, GSE107278;

FIG. 10G graphically illustrates data showing mevalonate/cholesterol synthesis pathway GSVA scores from RNAseq analysis of indicated memory P14 CD8 T cells, GSE182276. SG, salivary gland. WAT, white adipose tissue;

FIG. 10H graphically illustrates data showing PAGERANK™ scores and gene expression of Srebp2 and Runx3 in memory P14 CD8 T cells, GSE182276; and

FIG. 10I illustrates images of representative detection of Srebp2 and CD103 by immunofluorescence of congenically labeled memory P14 CD8 T cells; as described in detail in Example 1, below.

FIG. 11A-H illustrate data showing that a Srebp2-dependent metabolic program controls SI T_{RM} formation:

FIG. 11A-C schematically and graphically illustrate the ratio of transduced transferred P14 CD8 T cells (FIG. 11A and FIG. 11B), or P14 Cas9eGFP CD8 T cells (FIG. 11B), harvested from indicated tissues at indicated times after LCMV (FIG. 11A and FIG. 11B), or LM-GP33-41 (FIG. 11C) infection, evaluated by flow cytometry;

FIG. 11D schematically and graphically illustrates data showing the number of total Tet⁺ cells in the spleen, and total Tet⁺ T_{RM} cells in the SI, kidney, and liver of Scap^{fl/fl} Cd4-Cre⁺ and Scap^{wt} Cd4-Cre⁺ at indicated times after LCMV infection;

FIG. 11E graphically illustrates data showing LCMV titers measured by qPCR in the SI of WT and Scap knock-out (KO) mice previously infected with LM-GP_{33-41 3} days after LCMV rechallenge. NP, nucleoprotein. GP, glycoprotein;

FIG. 11F schematically and graphically illustrates data showing the number of total P14 CD8 T cells isolated from indicated tissues at days 7 and 21 after LCMV infection in mice treated with vehicle or lovastatin;

FIG. 11G graphically illustrates data showing T_{RM} scores of human CD8 T cells of the blood and SI profiled by scRNAseq grouped by statin use; and

FIG. 11H schematically and graphically illustrates data showing the ratio of total P14 CD8 T cells from indicated tissues in mice fed high versus low-cholesterol-containing diet,
as described in detail in Example 1, below.

FIG. 12A-F illustrate data showing that non-steroidal products of the mevalonate/cholesterol synthesis pathway mediate T_{RM} adaptations: FIG. 12A graphically illustrates an unsupervised hierarchical clustered heatmap of gene essentiality scores for genes related to the mevalonate/cholesterol synthesis pathway in the cellular populations profiled in our in vivo CRISPR/Cas9-mediated loss-of-function screen; blue and red denote positive and negative regulators of memory CD8 T cell formation, respectively;

FIG. 12B-C graphically illustrate data showing the rRatio of indicated transduced transferred P14 Cas9eGFP CD8 T cells, sgHpd (FIG. 12B) and sgFdft1 (FIG. 12C), harvested from indicated tissues at indicated times after LCMV infection, evaluated by flow cytometry;

FIG. 12D schematically and graphically illustrates an exemplary targeted in vivo CRISPR/Cas9-mediated loss-of-function screen of mediators of the effect of Fdft1 deletion on CD8 T cell memory formation, data visualization represents the enrichment as Log₂ FC of sgRNA frequencies in sgCD19 outputs versus sgCd19 input (x axis), and sgFdft1 outputs versus sgFdft1 input (y axis), for each effector and memory CD8 T cell subset;

FIG. 12E graphically illustrates data showing the ratio of transduced transferred P14 CD8 T cells harvested from indicated tissues at indicated times after LCMV infection, evaluated by flow cytometry; and

FIG. 12F graphically illustrates data showing total cell numbers of Empty and Pdss2 OE SI IEL P14 CD8 T cells in mice fed low or high cholesterol-containing diets,
as described in detail in Example 1, below.

FIG. 13A-H illustrate showing that Pdss2 expression is required and sufficient to promote mitochondrial respiration and CoQ synthesis in CD8 T cells:

FIG. 13A graphically illustrates oxygen consumption rate (OCR) of in vitro activated shCd19 and shSrebf2 CD8 T cells subjected to the MITOSTRESS™ test (Seahorse), oligomycin (O), FCCP (F), Rotenone/Antimycin A (R/A);

FIG. 13B graphically illustrates Oxygen consumption rate (OCR) of in vitro activated CD8 T cells treated with statin (simvastatin) in combination with mevalonolactone (MVA) or cholesterol (Chol.) and subjected to the MITOSTRESS™ tests (Seahorse);

FIG. 13C graphically illustrates OCR of in vitro-activated sgCd19 and sgPdss2 Cas9eGFP CD8 T cells subjected to the MITOSTRESS™ test (Seahorse);

FIG. 13D graphically illustrates OCR of in vitro activated sgCd19 and sgFdft1 Cas9eGFP CD8 T cells subjected to the MITOSTRESS™ test (Seahorse);

FIG. 13E graphically illustrates OCR of in vitro activated sgCd19, sgFdft1, and sgFdft1/Pdss2 Cas9eGFP CD8 T cells subjected to the MITOSTRESS™ test (Seahorse);

FIG. 13F graphically illustrates OCR of in vitro activated empty vector control (Empty) and Pdss2 OE CD8 T cells subjected to the MITOSTRESS™ test (Seahorse);

FIG. 13G graphically illustrates quantification of CoQ and demethoxyubiquinone (DMQ) species in in vitro activated sgCd19 and sgFdft1 Cas9eGFP CD8 T cells normalized to total protein content; and

FIG. 13H graphically illustrates quantification of CoQ and DMQ species in in vitro activated Empty and Pdss2 OE CD8 T cells normalized to total protein content, as described in detail in Example 1, below.

FIG. 14A-M illustrate data showing that increased production of non-steroidal products of the mevalonate/cholesterol synthesis pathway is a common requirement for SI T_{RM} and TIL:

FIG. 14A schematically illustrates an exemplary targeted in vivo screen of mevalonate/cholesterol synthesis pathway genes for SI T_{RM} formation in LCMV infection, and CD8 T cell accumulation in dLN and MC38-GP₃₃₋₄₁ tumors;

FIG. 14B schematically illustrates a Venn diagram of significant genes (fdr<0.01 for dLN and TIL, fdr<0.01 and Log₂ FC<−2 for SI T_{RM}) from (FIG. 14A);

FIG. 14C graphically illustrates Log₂ FC of sgRNA frequencies for each screen from FIG. 14A;

FIG. 14D schematically illustrates a metabolic pathway diagram displaying genes required for SI T_{RM} and TIL formation;

FIG. 14E graphically illustrates a ratio of transduced transferred P14 CD8 T cells harvested from the spleen and MC-38-GP₃₃₋₄₁ tumors, evaluated by flow cytometry 5-7 days after adoptive cell transfer;

FIG. 14F graphically illustrates ratio of KO P14 Cas9eGFP CD8 T cells vs control (sgCd19) in MC38-GP₃₃₋₄₁ tumors 7 days after adoptive cell transfer;

FIG. 14G graphically illustrates the ratio of Pdss2 OE vs Empty P14 CD8 T cells in the spleen and MC38-GP₃₃₋₄₁ tumors 5-7 days after adoptive cell transfer;

FIG. 14H graphically illustrates MC38-GP₃₃₋₄₁ tumor growth curves of mice receiving indicated transduced P14 Cas9eGFP CD8 T cells;

FIG. 14I graphically illustrates cell frequencies and total numbers of transduced P14 Cas9eGFP CD8 T cells in MC38-GP₃₃₋₄₁ tumors, dLN, and spleens 4 days after adoptive cell transfer;

FIG. 14J graphically illustrates B16-GP₃₃₋₄₁ tumor growth curves in mice adoptively transferred with indicated transduced P14 CD8 T cells;

FIG. 14K graphically illustrates cell frequencies in Empty and Pdss2 OE P14 CD8 T cells in MC38-GP₃₃₋₄₁ tumors;

FIG. 14L graphically illustrates B16-GP₃₃₋₄₁ tumor growth curves of mice receiving vehicle control, ZAA, αPD-1, or ZAA and αPD-1; and

FIG. 14M schematically illustrates a proposed model of the Srebp2-dependent metabolic programming of SI T_{RM} and TIL, as described in detail in Example 1, below.

FIG. 15A-F illustrate functional genetics in vivo, metabolomics and transcriptional analysis of ex vivo populations of memory CD8 T cells identify a graded upregulation of a Srebp2-dependent metabolic programming across T_{RM}:

FIG. 15A graphically illustrate an unsupervised hierarchical clustering heatmap of scaled GSVA scores for metabolic signatures across samples, p.i., post-infection;

FIG. 15B graphically illustrates sgRNA library heterogeneity reported as Gini Index values;

FIG. 15C graphically illustrates an upset plot showing intersection sizes and set sizes for positive and negative regulators of each P14 CD8 T cell population in the in vivo CRISPR/Cas9-mediated loss-of-function screen;

FIG. 15D schematically illustrates an exemplary sample acquisition and analysis workflow for LC-MS/MS-based untargeted metabolomics of ex vivo populations of CD8 T cells in the context of LCMV infection, gating strategy included for each cell type analyzed;

FIG. 15E illustrates a tandem mass spectra matching against commercial standards of mevalonate and mevalonolactone; tandem mass spectra derived from the most abundant ion for each compound; black spectra are derived from cell lysate, red (lighter, the lower) mirrored spectra from commercial standard; and

FIG. 15F graphically illustrates unsupervised hierarchical clustering of scaled relative abundances of identified annotated metabolites of P14 cells profiled by untargeted metabolomics at day 13 post LCMV infection, as described in detail in Example 1, below.

FIG. 16A-H illustrate upregulation of the mevalonate/cholesterol synthesis pathway in mouse and human T_{RM}: FIG. 16A graphically illustrates mevalonate/cholesterol synthesis GSVA scores from averaged single cell expression of spleen and SI P14 at indicated time points after LCMV infection profiled by scRNASeq, GSE131847;

FIG. 16A graphically illustrates mevalonate/cholesterol synthesis GSVA scores for indicated subsets of CD8 T cells in the spleen or SI in the context of LCMV infection, GSE157072;

FIG. 16C graphically illustrates mevalonate/cholesterol synthesis GSVA scores for indicated subsets of CD8 T cells at the indicated tissues in the context of LCMV and Herpes Simplex Virus (HSV), GSE70813;

FIG. 16D graphically illustrates Gene expression of indicated genes in P14 CD8 T cell populations from indicated tissues greater than (>) 30 days after LCMV infection, GSE182276;

FIG. 16E graphically illustrates mevalonate/cholesterol synthesis pathway scores for all human immune cells grouped by indicated tissues (left), cell types (middle), or cell types of the Jejunum SI (right), from the tissue immune cell scRNAseq atlas;

FIG. 16F graphically illustrates expression of selected genes on human CD8 T cells from PBMC, rectum, and SI samples, pooled from 13 healthy donors, GSE125527;

FIG. 16G illustrates immunofluorescence staining images of HMG-CoA reductase (HMGCR) protein expression in CD8 T cells from human spleen, SI, and colon by immunofluorescence staining, and quantification of total HMGCR by CD8 T cell area; and

FIG. 16H graphically illustrates GSEA of the mevalonate/cholesterol synthesis in Runx3 KO or control CD8 T cells GSE106107, as described in detail in Example 1, below.

FIG. 17A-J illustrates data showing that a Srebp2-dependent metabolic program controls SI T_{RM} formation:

FIG. 17A graphically illustrates quantification of gene expression by qPCR of shRNAmir control (shCd19) or shSrebf2 shRNAmir P14 CD8 T cells before adoptive transfer;

FIG. 17B graphically illustrates data showing the frequency of effector shRNAmir control (shCd19) or shSrebf2 shRNAmir P14 CD8 T cell populations in the spleen 7 days after LCMV infection;

FIG. 17C graphically illustrates data showing the frequency of memory shRNAmir control (shCd19) or shSrebf2 shRNAmir P14 CD8 T cell populations in the spleen 21 days after LCMV infection;

FIG. 17D graphically illustrates data showing CRISPR/Cas9-mediated indel efficiency of the Srebf2 sgRNA construct on sorted transduced P14 cells before adoptive transfer;

FIG. 17E graphically illustrates data showing the frequency of effector sgCd19 or sgSrebf2 P14 cell populations in the spleen 7 days after LCMV infection;

FIG. 17F graphically illustrates data showing the frequency of memory sgCd19 or sgSrebf2 P14 cell populations in the spleen 21 days after LCMV infection;

FIG. 17G graphically illustrates data showing total cell quantification of CD44^{high} CD8 T cells (left) and Tet⁺ CD8 T cells (right) in the blood of Scap WT or Scap KO at different time points after LCMV infection;

FIG. 17H graphically illustrates data showing total cell quantification of Tet⁺ CD8 T cells (upper) and CD44^{high} CD8 T cells (lower) in SI, kidney, and liver of Scap WT or Scap KO at different time points after LCMV infection;

FIG. 17I graphically illustrates data showing the ratio of congenically distinct P14 CD8 T cells transduced with a constitutively active nuclear form of human Srebp2⁵⁶ (nSrebp2) or an empty vector (Empty) from indicated tissues 7 days after LCMV infection; and

FIG. 17J graphically illustrates data from a flow cytometry analysis of cholesterol content by Filipin stain (left), and LDLR expression in P14 CD8 T cells transduced with a bicistronic construct encoding Thy1.1 alone or Thy1.1 and nSrebp2 (right); P14 CD8 T cells from the kidney, liver, WAT, and SI were gated on the IV⁻ population (FIG. 17H and FIG. 17I), as described in detail in Example 1, below.

FIG. 18A-F illustrates data showing the limited effect of Srebp2 on transcriptional programs of CD8 T cell differentiation: FIG. 18A graphically illustrates data showing Srebf2 expression values from the bulk ULI-RNAseq analysis of shRNAmir control (shCd19) and Srebf2 shRNAmir P14 CD8 T cells from the spleen, kidney, liver, and SI from day 7 and 14 after LCMV infection;

FIG. 18B graphically illustrates data showing mevalonate/cholesterol synthesis pathway GSVA scores from the bulk ULI-RNAseq analysis of shRNAmir control (shCd19) and Srebf2 shRNAmir P14 CD8 T cells from the spleen, kidney, liver, and SI from day 7 and 14 after LCMV infection;

FIG. 18C graphically illustrates data showing unsupervised hierarchically clustered heatmap of mevalonate/cholesterol synthesis pathway gene expression values from the bulk ULI-RNAseq analysis of shRNAmir control (shCd19) and Srebf2 shRNAmir P14 CD8 T cells from the spleen, kidney, liver, and SI from day 7 and 14 after LCMV infection;

FIG. 18D graphically illustrates data showing core T_{RM} GSVA scores from the bulk ULI-RNAseq analysis of shRNAmir control (shCd19) and Srebf2 shRNAmir P14 CD8 T cells from the spleen, kidney, liver, and SI from day 7 and 14 after LCMV infection;

FIG. 18E graphically illustrates data showing circulating GSVA scores from the bulk ULI-RNAseq analysis of shRNAmir control (shCd19) and Srebf2 shRNAmir P14 CD8 T cells from the spleen, kidney, liver, and SI from day 7 and 14 after LCMV infection;

FIG. 18F graphically illustrates data showing volcano plots of differentially expressed genes between shCd19 and shSrebf2 SI P14 CD8 T cells at days 7 and 14 after LCMV infection, adjusted (adj) p-value. P14 CD8 T cells from the kidney, liver and SI were gated on the IV⁻ population (a-f). T_{E} were defined as KLRG1^{high}CD127^{low}P14 CD8 T cells,
as described in detail in Example 1, below.

FIG. 19A-L illustrates data showing SI T_{RM} uptake and sense dietary cholesterol:

FIG. 19A graphically illustrates data showing quantification by flow cytometry of in vivo incorporation of BODIPY (4,4-difluoro-4-bora-3a,4a-diaza-s-indacene)-cholesterol on indicated cell populations after 10 minutes after oral administration of BODIPY-cholesterol to memory P14 mice;

FIG. 19B graphically illustrates data showing quantification by flow cytometry of incorporation of Bodipy-LDL on indicated cell types of the spleen 20 minutes (min) after IV delivery to memory P14 mice;

FIG. 19C graphically illustrates data showing quantification of intracellular cholesterol by filipin staining in Tet⁺ populations of CD8 T cells for various tissues greater than (>) 30 days post LCMV infection measured by imaging flow cytometry;

FIG. 19D graphically illustrates data showing expression analysis by qPCR of selected Srebp2 targets on CD8 T cells from the spleen and SI of mice on regular chow or subjected to low and high cholesterol-containing diets;

FIG. 19E schematically illustrates an exemplary experimental design to measure transcriptional changes induced by dietary cholesterol on established SI T_{RM} by ULI-RNAseq;

FIG. 19F graphically illustrates data showing SI T_{RM} frequencies from total CD8 T cells after 7 days of dietary intervention;

FIG. 19G graphically illustrates data showing food consumption per day per mouse subjected to a low or high-cholesterol diet;

FIG. 19H graphically illustrates data showing bodyweight of mice subjected to low or high cholesterol-containing diets;

FIG. 19I graphically illustrates data showing total cholesterol in serum in mice subjected to low or high cholesterol-containing diets;

FIG. 19J graphically illustrates data showing a volcano plot of differentially expressed genes in SI T_{RM} from mice fed a high vs a low cholesterol-containing diet;

FIG. 19K graphically illustrates data showing mevalonate/cholesterol synthesis GSVA scores in SI T_{RM} from mice fed a high versus low cholesterol-containing diet; and

FIG. 19L illustrates a table with data showing gene pathway analysis of top upregulated and top downregulated genes between SI T_{RM} from mice fed a high versus low cholesterol-containing diet (MsigDb, Hallmark gene sets); P14 CD8 T cells and CD8β⁺/CD44^{high}/Tet⁺ from the kidney, liver and SI were gated on the IV population (FIG. 19A, FIG. 19B, FIG. 19D, FIG. 19E, FIG. 19F, FIG. 19J, FIG. 19K, and FIG. 19I), as described in detail in Example 1, below.

FIG. 20A-H illustrates data showing that mevalonate/cholesterol synthesis pathway intermediates sustain the production of non-steroidal metabolites:

FIG. 20A schematically illustrates the mevalonate/cholesterol synthesis pathway with adjacent and downstream metabolic routes; enzymes not included in the first CRISPR/Cas9-mediated loss-of-function screen in grey, downstream products and/or fates of cholesterol are boxed, key measured metabolites in bold, non-steroidal metabolites in blue, key enzymes are bold and colored by in vivo effect of deletion in P14 CD8 T cells, gain-of-function in red, loss-of-function in blue, dashed lines indicate indirect or unknown mode of regulation;

FIG. 20B graphically illustrates data showing the abundance of 4-hydroxybenzaldehyde profiled by LC-MS/MS of ex vivo populations of P14 cells 13 days after LCMV infection;

FIG. 20C graphically illustrates data showing tandem mass spectra matching against commercial standards of 4-hydroxybenzaldehyde;

FIG. 20D graphically illustrates data showing CRISPR/Cas9-mediated indel efficiency of the Hpd sgRNA construct on sorted transduced P14 cells before adoptive transfer;

FIG. 20E graphically illustrates data showing CRISPR/Cas9-mediated indel efficiency of the Fdft1 sgRNA construct on sorted transduced P14 CD8 T cells before adoptive transfer;

FIG. 20F graphically illustrates data showing Gini index scores of sgRNA representation in each library;

FIG. 20G is a schematic illustration of the contribution of mevalonate/cholesterol pathway intermediates to non-steroidal products; and

FIG. 20H graphically illustrates data showing quantification of Pdss2 mRNA by qPCR in transduced Empty and Pdss2 OE P14 CD8 T cells,
as described in detail in Example 1, below.

FIG. 21A-M illustrates data showing that increased production of non-steroidal products of the mevalonate/cholesterol synthesis pathway is a common requirement between SI T_{RM} and TIL:

FIG. 21A graphically illustrates a dimensionality reduction plot by UMAP of P14 CD8 T cells profiled from spleens and subcutaneously implanted MC38-GP₃₃₋₄₁ tumors by scRNAseq 7 days after adoptive cell transfer;

FIG. 21B graphically illustrates data showing mevalonate/cholesterol synthesis signature values and expression of indicated genes in P14 CD8 T cells from the spleen and MC38-GP₃₃₋₄₁ tumors profiled by scRNAseq. MAGIC-imputed gene values are shown;

FIG. 21C illustrates a hierarchically clustered heatmap of scaled averaged gene expression values by cluster of P14 cells profiled from spleens and MC38-GP₃₃₋₄₁ tumors by scRNAseq 7 days after adoptive transfer;

FIG. 21D graphically illustrates a dimensionality reduction plot by UMAP of CD8 T cell metaclusters (left) with “meta.cluster.coarse” labels and hierarchically clustered heatmap of mevalonate/cholesterol synthesis pathway scores grouped by “meta.cluster.coarse” labels and cell type: NPC, nasopharingeal carcinoma, ESCA, esophageal cancer, LC, lung cancer, HCC, hepatocellular carcinoma, CHOL, cholangiocarcinoma, RC, renal carcinoma, CRC, colorectal cancer, AML, acute myeloid leukemia, BCL, B-cell lymphoma, MM, multiple myeloma, HNSCC, head and neck squamous cell carcinoma, THCA, thyroid carcinoma, MELA, melanoma, BCC, basal cell carcinoma, SCC, squamous cell carcinoma, BRCA, breast cancer, STAD, stomach adenocarcinoma, PACA, pancreatic cancer, OV, ovarian cancer, FTC, fallopian tube carcinoma, UCEC, uterine corpus endometrial carcinoma. EMRA, effector memory CD45RA⁺, mem, memory;

FIG. 21E graphically illustrates data showing sgRNA library heterogeneity reported as Gini Index values;

FIG. 21F graphically illustrates data showing CRISPR/Cas9-mediated indel efficiency of the Pdss2 sgRNA construct on sorted transduced P14 CD8 T cells;

FIG. 21G graphically illustrates data showing frequency of PD1^{high}Tim3^{high} and PD1^{high}Tim3^{low} subpopulations for sgCd19 and sgFdft1 Cas9 P14 CD8 T cells in MC38-GP₃₃₋₄₁ tumors at day 5-7 after adoptive transfer;

FIG. 21H graphically illustrates data showing frequency of PD1^{high}Tim3^{high} and PD1^{high}Tim3^{low} subpopulations for Empty and Pdss2 OE P14 CD8 T cells in MC38-GP₃₃₋₄₁ tumors at day 5-7 after adoptive transfer;

FIG. 21I graphically illustrates data showing quantification of gene expression by qPCR of control (shNT) and Fdft1 deficient (shFdft1) P14 CD8 T cells before adoptive transfer;

FIG. 21J graphically illustrates data showing the restimulation capacity of control (shNT) and Fdft1 deficient (shFdft1) P14 TIL 4 days after in vivo transfer treated with GP₃₃₋₄₁ peptide ex vivo;

FIG. 21K graphically illustrates data showing MC38-GP₃₃₋₄₁ tumor growth curves of mice receiving ZAA or vehicle;

FIG. 21L graphically illustrates data showing survival of mice implanted with MC38-GP₃₃₋₄₁ tumors receiving ZAA or vehicle; and

FIG. 21M graphically illustrates data showing MC38-GP₃₃₋₄₁ tumor growth curves of mice receiving ZAA in combination with CD8 T cell depletion,
as described in detail in Example 1, below.

FIG. 22A-C illustrates data showing the requirement for the mevalonate/cholesterol synthesis enzymes for TIL persistence and heightened antitumor activity in a melanoma model:

FIG. 22A graphically illustrates data showing GSEA of the mevalonate/cholesterol synthesis pathway in genes pre-ranked by their impact on TIL accumulation in B16-OVA tumors from a published CRISPR/Cas9-mediated loss-of-function screen;

FIG. 22B illustrates a dimensionality reduction plot by UMAP of WT and Regnase-1 KO OT-I cells profiled from MC38-GP₃₃₋₄₁ tumors by scRNAseq 7 days after adoptive transfer (left) and unsupervised hierarchical clustering heatmap of selected genes related to CD8 T cell cytotoxicity (Ifng) stemness (Tcf7), proliferation (Mki67), and the mevalonate/cholesterol synthesis pathway (Srebf2, Hmgcs1, Fdft1, Hmgcr, and Ldlr) grouped by cell type and cluster; and

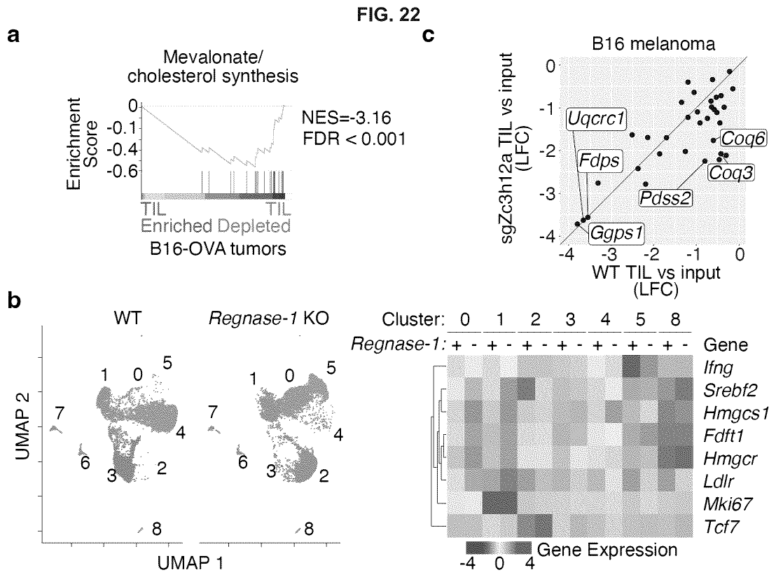

FIG. 22C graphically illustrates data showing the comparison of the effect of targeting the 50 genes of the mevalonate/cholesterol pathway included in our targeted screen in WT TIL or Regnase-1 KO TIL (sgZc3h12a) from a published CRISPR/Cas9-mediated loss-of-function screen in B16 melanoma tumors³²,
as described in detail in Example 1, below.

Like reference symbols in the various drawings indicate like elements.

### DETAILED DESCRIPTION

In alternative embodiments, provided are methods for metabolically enhancing CAR-T cell or genetically engineered CD8 T cell function in vitro and in vivo, and their persistence in vivo, wherein the enhancing of CAR-T cells can be used for the enhancement of CAR-T cell or genetically engineered CD8 T cell efficacy in cancer immunotherapy.

Provided herein are methods for enhancing the ability of chimeric antigen receptor (CAR)-CD8 T cells or genetically engineered CD8 T cells to persist longer in solid tumors, and thus, provide enhanced protection in cell-based immunotherapies, by inhibiting a squalene synthase (or Fdft1), an enzyme of the mevalonate/cholesterol synthesis pathway. The squalene synthase (or Fdft1) can be inhibited genetically or pharmacologically. In alternative embodiments, methods as provided herein comprise use of CAR-T cells or genetically engineered CD8 T cells having genetically deleted or disabled squalene synthase (or Fdft1). In alternative embodiments, methods as provided herein comprise use of CAR-T cells or genetically engineered CD8 T cells that have been treated in vivo or ex vivo (before being administered in vivo) with an agent, which for example can be an inhibitory nucleic acid or a small molecule inhibitor, to delete or diminish the activity of a squalene synthase (or Fdft1) in the CAR-T cell or genetically engineered CD8 T cell.

Described herein for the first time is an actionable metabolic target of the mevalonate/cholesterol synthesis pathway that can be leveraged to enhance CAR-T cell or genetically engineered CD8 T cell function at three different stages of cell-based immunotherapy using both pharmacological and genetic approaches; at least three applications are provided herein:
1) Improve CAR-T cell or genetically engineered CD8 T cell therapies and products by using an Fdft1 inhibitor during the CAR-T cell or genetically engineered CD8 T cell manufacture process,
2) Improve CAR-T cell or genetically engineered CD8 T cell products by genetically deleting Fdft1 gene before therapeutic administration of the CAR-T cell, or otherwise genetically modifying the Fdft1 gene in the CAR-T cell or genetically engineered CD8 T cell, or otherwise expressing a nucleic acid (for example, an Fdft1 miRNA or Fdft1 antisense nucleic acid) capable of modifying expression of a Fdft1 gene, such that the CAR-T cell or genetically engineered CD8 T cell has no or substantially no Fdft1 enzyme activity; and/or
3) Provide continued Fdft1 inhibition after CAR-T cell or genetically engineered CD8 T cell product infusion, where the continued Fdft1 inhibition can be in form of an administered nucleic acid, polypeptide or small molecule Fdft1 inhibitor, such as mevastatin or zaragozic acid A (ZAA), or (see, for example, Ichikawa et al, ACS Med. Chem. Lett. 2013, 4, 10, 932-936):

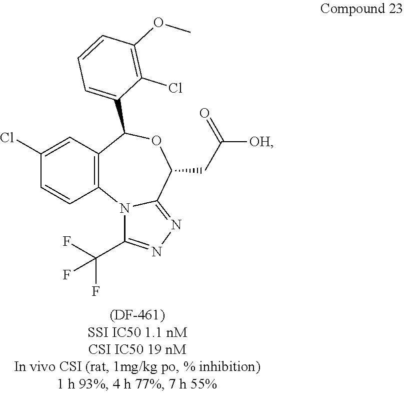 [SMILES: COc1cccc([C@H]2O[C@H](CC(=O)O)c3nnc(C(F)(F)F)n3-c3ccc(Cl)cc32)c1Cl]

or, bavachinin.

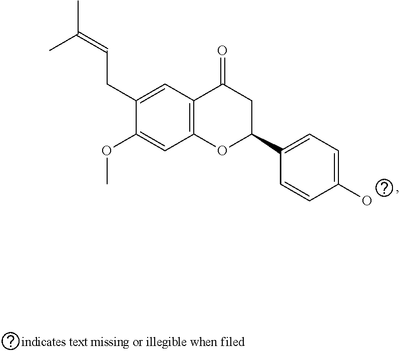 [SMILES: COc1cc2c(cc1CC=C(C)C)C(=O)C[C@@H](c1ccc(O)cc1)O2]

It has been demonstrated that genetically targeting the Fdft1 enzyme in CD8 T cells induces their ability to reside in tissues by increasing the production of non-steroidal products of the mevalonate/cholesterol synthesis pathway, including coenzyme Q. In addition, a fungal-derived metabolite, Zaragozic Acid A, which is a previously published competitive inhibitor of Fdft1, can be used to treat T cells during CAR-T cell manufacturing, or after CAR-T cell product infusion, which provides an enhancement on the ability of CAR-T cells to persist in the tumor.

As discussed in Example 1, below, we found that memory CD8 T cells deployed a range of adaptations to tissue residency, including reliance on non-steroidal products of the mevalonate/cholesterol pathway, such as Coenzyme Q 15 (CoQ), driven by increased activity of the transcription factor Srebp2. This metabolic adaptation was most pronounced in the small intestine (SI), where T_{RM} interface with dietary cholesterol and maintain a heightened state of activation⁴, and was shared by functional TIL in diverse tumor types in mice and humans. Enforcing CoQ synthesis through Fdft1 deletion or Pdss2 overexpression promoted mitochondrial respiration, memory formation upon viral infection, and enhanced antitumor immunity. In sum, through a systematic exploration of T_{RM} metabolism, we reveal how these programs can be leveraged to fuel CD8 T cell memory formation in the context of acute infections and enhance antitumor immunity.

This data further demonstrates a common dependency on mevalonate/cholesterol synthesis enzymes for SI T_{RM} formation and TIL accumulation. Based on these observations, we found two actionable enzymes, Fdft1 and Pdss2, which can be manipulated to enforce CoQ production, enhance the mitochondrial respiratory capacity of CD8 T cells, and promote in vivo accumulation of CD8 T cells in the context of infection and tumors. Whether these adaptations are conserved across all tumor types and contexts remains to be further investigated. The fact that loss of most genes of the mevalonate/cholesterol synthesis pathway impaired TIL accumulation in the melanoma model B16-OVA from a previous study, suggests these observations may be generalized to other tumor contexts.

### CAR T Cell Production and Administration

CAR T-cells can be produced using any known protocol, for example, see for example U.S. Pat. Nos. 11,485,750; 11,472,858; 11,471,490; 11,471,519; and U.S. patent application serial no. US 2023 0331864 A1; US 2023 0332131 A1 and US 2023 0331855 A1.

For example, the first step in the production of CAR T-cells is the isolation of T cells from human blood. CAR T-cells may be manufactured either from the patient's own blood, known as an autologous treatment, or from the blood of a healthy donor, known as an allogeneic treatment. The manufacturing process is the same in both cases; only the choice of initial blood donor is different.

Leukocytes can be isolated using a blood cell separator in a process known as leukocyte apheresis. Peripheral blood mononuclear cells (PBMCs) are then separated and collected. The products of leukocyte apheresis are then transferred to a cell-processing center, where specific T cells are stimulated so that they will actively proliferate and expand to large numbers. To drive their expansion, T cells can be typically treated ex vivo with the cytokine interleukin 2 (IL-2) and anti-CD3 antibodies. In alternative embodiments, the T cells are treated with a squalene synthase (or Fdft1) at this stage of the process.

Expanded T cells are then purified and then transduced with a gene encoding the engineered CAR via a retroviral vector, typically either an integrating gammaretrovirus (RV) or a lentiviral (LV) vector. These vectors are modified to be safe, for example, to have a partial deletion of their U3 region. Editing tools such as CRISPR/Cas9 can be used instead of retroviral vectors to integrate the CAR gene into specific sites in the genome. In alternative embodiments, the T cells are also genetically altered to have deleted or decreased ability to express squalene synthase (or Fdft1), or have diminished squalene synthase (or Fdft1) activity. The genetic sequences used to delete or modify squalene synthase (or Fdft1) gene can be on the same or different vectors or editing tools (for example, CRISPR/Cas9) as the engineered CAR.

The patient can be lymphodepleted, for example, by chemotherapy, prior to the introduction of the engineered CAR T-cells or genetically engineered T cells as provided herein. The depletion of the number of circulating leukocytes in the patient can upregulate the number of cytokines that are produced and reduces competition for resources, which helps to promote the expansion of the engineered CAR T-cells. In alternative embodiments, before, in conjunction with, and/or after in vivo administration of the engineered CAR T-cells, inhibitors of squalene synthase (or Fdft1) nucleic acid and/or protein (enzyme) are also administered to the individual.

Any new or known CAR T cell can be used to practice methods as provided herein, for example, tisagenlecleucel (or KYMRIAH™, Novartis) axicabtagene ciloleucel (or YESCARTA™, Kite Pharma/Gilead), lisocabtagene maraleucel (or BREYANZI™, Juno Therapeutics/BMS), idecabtagene vicleucel (or ABECMA™ Bluebird Bio/BMS), and the like.

### Therapies

In alternative embodiments, CAR T cells or genetically engineered T cells, or formulations as provided herein, are used in practicing methods as provided herein are used to treat cancers or tumors, for example, where the CAR T cells can eradicate a certain cell population, for instance, a cancer such as a lymphoma, or any tumor, including solid tumors, or a non-solid tumor such as a blood cancer.

In alternative embodiments, CAR T cells or genetically engineered T cells used in practicing methods as provided herein are used to treat or ameliorate an autoimmune disease. For example, T cells can also mediate tolerance to antigens, and a regulatory T cell genetically modified to express a CAR could have the potential to confer tolerance to a specific antigen, a property that can be utilized in organ transplantation or rheumatic diseases like lupus.

In alternative embodiments, CAR T cells or genetically engineered T cells and provided herein, and CAR T cells or genetically engineered T cells used in practicing methods as provided herein are genetically modified to express no, substantially no, or less than wild type levels of Fdft1.

In alternative embodiments, the Fdft1 gene, such as a human Fdft1 gene, or Fdft1 message (mRNA) is targeted with antisense or miRNA or equivalent sequences, which can be designed using the sequences:

Human Fdft1
(SEQ ID NO: 1) Nucleotide Sequence (1254 nt):
ATGGAGTTCGTGAAATGCCTTGGCCACCCCGAAGAGTTCTACAACCTGGT
GCGCTTCCGGATCGGGGGCAAGCGGAAGGTGATGCCCAAGATGGACCAG
GACTCGCTCAGCAGCAGCCTGAAAACTTGCTACAAGTATCICAATCAGAC
CAGTCGCAGITTCGCAGCTGTTATCCAGGCGCTGGATGGGGAAATGCGCA
ACGCAGTGTGCATATTTTATCTGGTTCTCCGAGCTCTGGACACACTGGAAG
ATGACATGACCATCAGTGTGGAAAAGAAGGTCCCGCTGTTACACAACTTT
CACTCTTTCCTTTACCAACCAGACTGGCGGTTCATGGAGAGCAAGGAGAA
GGATCGCCAGGTGCTGGAGGACTTCCCAACGATCTCCCTTGAGTTTAGAA
ATCTGGCTGAGAAATACCAAACAGTGATTGCCGACATTTGCCGGAGAATG
GGCATTOGGATGGCAGAGTTTTTGGATAAGCATGTGACCICTGAACAGGA
GTGGGACAAGTACTGCCACTATGTTGCTGGGCTGGTCGGAATTGGCCTTTC
CCGTCTTTTCTCAGCCTCAGAGTTTGAAGACCCCTTAGTTGGTGAAGATAC
AGAACGTGCCAACTCTATGGGCCTGTTTTTGCAGAAAACAAACATCATCC
GTGACTATCTGGAAGACCAGCAAGGAGGAAGAGAGTTCTGGCCTCAAGA
GGTTTGGAGCAGGTATGTTAAGAAGTTAGGGGATTTTGCTAAGCCGGAGA
ATATTGACTTGGCCGTGCAGTGCCTGAATGAACTTATAACCAATGCACTGC
ACCACATCCCAGATGTCATCACCTACCTTTCGAGACTCAGAAACCAGAGT
GTGTTTAACTTCTGTGCTATTCCACAGGTGATGGCCATTGCCACTTTGGCT
GCCTGTTATAATAACCAGCAGGTGTTCAAAGGGGCAGTGAAGATTCGGAA
AGGGCAAGCAGTGACCCTGATGATGGATGCCACCAATATGCCAGCTGTCA
AAGCCATCATATATCAGTATATGGAAGAGATTTATCATAGAATCCCCGAC
TCAGACCCATCTTCTAGCAAAACAAGGCAGATCATCTCCACCATCCGGAC
GCAGAATCTTCCCAACTGTCAGCTGATTTCCCGAAGCCACTACTCCCCCAT
CTACCTGTCGTTTGTCATGCTTTTGGCTGCCCTGAGCTGGCAGTACCTGAC
CACTCTCTCCCAGGTAACAGAAGACTATGTTCAGACTGGAGAACACTGA

In alternative embodiments, the Fdft1 protein activity is decreased (modified to below wild type levels) in vivo by administration of small molecules, proteins such as antibodies, or nucleic acids, which can be designed or manufactured using the sequence:

(SEQ ID NO: 2) Translation (417 aa):
MEFVKCLGHPEEFYNLVRFRIGGKRKVMPKMDQDSLSSSLKTCYKYLNQ
TSRSFAAVIQALDGEMRNAVCIFYLVLRALDTLEDDMTISVEKKVPLLH
NFHSFLYQPDWRFMESKEKDRQVLEDFPTISLEFRNLAEKYQTVIADIC
RRMQIGMAEFLDKHVTSEQEWDKYCHYVAGLVGIGLSRLFSASEFEDPL
VGEDTERANSMGLFLQKTNIIRDYLEDQQGGREFWPQEVWSRYVKKLGD
FAKPENIDLAVQCLNELITNALHHIPDVITYLSRLRNQSVENFCAIPQV
MAIATLAACYNNQQVFKGAVKIRKGQAVTLMMDATNMPAVKAIIYQYME
ETYHRIPDSDPSSSKTRQIISTIRTQNLPNCQLISRSHYSPIYLSFVML
LAALSWQYLTTLSQVTEDYVQTGEH

### Formulations and Pharmaceutical Compositions

In alternative embodiments, provided are compounds and compositions, including formulations and pharmaceutical compositions, for use in in vivo, in vitro or ex vivo methods for treating, ameliorating, preventing or reversing: a cancer, for example, a breast cancer or a thyroid cancer by administering an inhibitor of a squalene synthase or an Fdft1 inhibitor.

In alternative embodiments, the pharmaceutical compositions as provided herein or as used in methods as provided herein can be administered parenterally, topically, orally or by local administration, such as by aerosol or transdermally. In alternative embodiments, pharmaceutical compositions can be prepared in various forms, such as granules, tablets, pills, capsules, suspensions, taken orally, suppositories and salves, lotions and the like. Pharmaceutical formulations as provided herein may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, lozenges, gels, geltabs, on patches, in implants, etc. In practicing embodiments as provided herein, the pharmaceutical compounds can be delivered by transdermally, by a topical route, formulated as applicator sticks, solutions, suspensions, emulsions, gels, creams, ointments, pastes, jellies, paints, powders, and aerosols. Oral carriers can be elixirs, syrups, capsules, tablets, pills, geltabs and the like.

In alternative embodiments, provided are pharmaceutically acceptable salts of compounds as provided herein or as used in methods as provided herein, including pharmaceutically acceptable non-toxic bases or acids including inorganic or organic bases and inorganic or organic acids. In alternative embodiments, salts are derived from inorganic bases such as aluminum, ammonium, calcium, copper, ferric, ferrous, lithium, magnesium, manganic salts, manganese, potassium, sodium, zinc, and the like; or, salts can be in a solid form, or in a crystal structure, or the form of hydrates. In alternative embodiments, salts are pharmaceutically acceptable organic non-toxic bases including salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethyl aminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. In alternative embodiments, for example, if a compound provided herein is basic, salts are prepared from pharmaceutically acceptable non-toxic acids, including inorganic and organic acids. Such acids include acetic, benzenesulfonic, benzoic, camphorsulfonic, carbonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

In alternative embodiments, pharmaceutically acceptable salts include hemisalts of non-toxic acids or bases, or hemihydrates.

In alternative embodiments, compounds and compositions as provided herein, or a compound used to practice methods as provided herein, are delivered orally, for example, as pharmaceutical formulations for oral administration, and can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients can be carbohydrate or protein fillers, for example, sugars, including lactose, sucrose, mannitol, or sorbitol; starch from corn, wheat, rice, potato, or other plants; cellulose such as methyl cellulose, hydroxypropylmethyl-cellulose, or sodium carboxymethyl cellulose; and gums including arabic and tragacanth; and proteins, for example, gelatin and collagen. Disintegrating or solubilizing agents may be added, such as the cross-linked polyvinyl pyrrolidone, agar, alginic acid, or a salt thereof, such as sodium alginate.

In alternative embodiments, liquid carriers are used to manufacture or formulate compounds as provided herein, or a composition used to practice the methods as provided herein, including carriers for preparing solutions, suspensions, emulsions, syrups, elixirs and pressurized compounds. The active ingredient (for example, a composition as provided herein or as used in methods as provided herein) can be dissolved or suspended in a pharmaceutically acceptable liquid carrier such as water, an organic solvent, a mixture of both or pharmaceutically acceptable oils or fats. The liquid carrier can comprise other suitable pharmaceutical additives such as solubilizers, emulsifiers, buffers, preservatives, sweeteners, flavoring agents, suspending agents, thickening agents, colors, viscosity regulators, stabilizers or osmo-regulators.

In alternative embodiments, solid carriers are used to manufacture or formulate compounds as provided herein, or a composition used to practice the methods as provided herein, including solid carriers comprising substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (for example, povidone, gelatin, hydroxypropylmethyl cellulose), lubricant, inert diluent, preservative, disintegrant (for example, sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

In alternative embodiments, concentrations of therapeutically active compound in a formulation can be from between about 0.1% to about 100% by weight.

In alternative embodiments, therapeutic formulations are prepared by any method well known in the art, for example, as described by Brunton et al., eds., Goodman and Gilman's: The Pharmacological Bases of Therapeutics, 12th ed., McGraw-Hill, 2011; Remington: The Science and Practice of Pharmacy, Mack Publishing Co., 20th ed., 2000; Avis et al., eds., Pharmaceutical Dosage Forms: Parenteral Medications, published by Marcel Dekker, Inc., N.Y., 1993; Lieberman et al., eds., Pharmaceutical Dosage Forms: Tablets, published by Marcel Dekker, Inc., N.Y., 1990; and Lieberman et al., eds., Pharmaceutical Dosage Forms: Disperse Systems, published by Marcel Dekker, Inc., N.Y., 1990.

In alternative embodiments, therapeutic formulations are delivered by any effective means appropriated for a particular treatment. For example, depending on the specific antitumor agent to be administered, the suitable means include oral, rectal, vaginal, nasal, pulmonary administration, or parenteral (including subcutaneous, intramuscular, intravenous and intradermal) infusion into the bloodstream. For parenteral administration, antitumor agents as provided herein may be formulated in a variety of ways. Aqueous solutions of the modulators can be encapsulated in polymeric beads, liposomes, nanoparticles or other injectable depot formulations known to those of skill in the art. In alternative embodiments, compounds and compositions as provided herein, or a compound used to practice methods as provided herein, are administered encapsulated in liposomes (see below). In alternative embodiments, depending upon solubility, compositions are present both in an aqueous layer and in a lipidic layer, for example, a liposomic suspension. In alternative embodiments, a hydrophobic layer comprises phospholipids such as lecithin and sphingomyelin, steroids such as cholesterol, more or less ionic surfactants such a diacetylphosphate, stearylamine, or phosphatidic acid, and/or other materials of a hydrophobic nature.

The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, for example, the latest edition of Remington's Pharmaceutical Sciences, Maack Publishing Co., Easton PA (“Remington's”). For example, in alternative embodiments, compounds and compositions as provided herein, or a compound used to practice methods as provided herein, are formulated in a buffer, in a saline solution, in a powder, an emulsion, in a vesicle, in a liposome, in a nanoparticle, in a nanolipoparticle and the like. In alternative embodiments, the compositions can be formulated in any way and can be applied in a variety of concentrations and forms depending on the desired in vivo, in vitro or ex vivo conditions, a desired in vivo, in vitro or ex vivo method of administration and the like. Details on techniques for in vivo, in vitro or ex vivo formulations and administrations are well described in the scientific and patent literature. Formulations and/or carriers used to practice embodiments as provided herein can be in forms such as tablets, pills, powders, capsules, liquids, gels, syrups, slurries, suspensions, etc., suitable for in vivo, in vitro or ex vivo applications.

In practicing embodiments as provided herein, the compounds (for example, formulations) as provided herein or as used in methods as provided herein can comprise a solution of compositions disposed in or dissolved in a pharmaceutically acceptable carrier, for example, acceptable vehicles and solvents that can be employed include water and Ringer's solution, an isotonic sodium chloride. In addition, sterile fixed oils can be employed as a solvent or suspending medium. For this purpose any fixed oil can be employed including synthetic mono- or diglycerides, or fatty acids such as oleic acid. In one embodiment, solutions and formulations used to practice embodiments as provided herein are sterile and can be manufactured to be generally free of undesirable matter. In one embodiment, these solutions and formulations are sterilized by conventional, well known sterilization techniques.

The solutions and formulations used to practice methods as provided herein can comprise auxiliary substances as required to approximate physiological conditions such as pH adjusting and buffering agents, toxicity adjusting agents, for example, sodium acetate, sodium chloride, potassium chloride, calcium chloride, sodium lactate and the like. The concentration of active agent in these formulations can vary widely, and can be selected primarily based on fluid volumes, viscosities and the like, in accordance with the particular mode of in vivo, in vitro or ex vivo administration selected and the desired results.

The compounds and compositions as provided herein, or a compound used to practice methods as provided herein, can be delivered by the use of liposomes. In alternative embodiments, by using liposomes, particularly where the liposome surface carries ligands specific for target cells or organs, or are otherwise preferentially directed to a specific tissue or organ type, one can focus the delivery of the active agent into a target cells in an in vivo, in vitro or ex vivo application.

The compounds and compositions as provided herein, or a compound used to practice methods as provided herein, can be directly administered, for example, under sterile conditions, to an individual (for example, a patient) to be treated. The modulators can be administered alone or as the active ingredient of a pharmaceutical composition. Compositions and formulations as provided herein can be combined with or used in association with other therapeutic agents. For example, an individual may be treated concurrently with conventional therapeutic agents.

### Nanoparticles, Nanolipoparticles and Liposomes

Provided are nanoparticles, nanolipoparticles, vesicles and liposomal membranes comprising compounds and compositions used to practice the methods and embodiments as provided herein. Provided are multilayered liposomes comprising compounds used to practice embodiments as provided herein, for example, as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice embodiments as provided herein.

Liposomes can be made using any method, for example, as described in Park, et al., U.S. Pat. Pub. No. 20070042031, including the method of producing a liposome by encapsulating an active agent (for example, compounds and compositions as provided herein, or a compound used to practice methods as provided herein), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice embodiments as provided herein comprise a substituted ammonium and/or polyanions, for example, for targeting delivery of a compound as provided herein, or a compound used to practice methods as provided herein, to a desired cell type or organ, for example, brain, as described for example, in U.S. Pat. Pub. No. 20070110798.

Provided are nanoparticles comprising compounds as provided herein, for example, used to practice methods as provided herein in the form of active agent-containing nanoparticles (for example, a secondary nanoparticle), as described, for example, in U.S. Pat. Pub. No. 20070077286. In one embodiment, provided are nanoparticles comprising a fat-soluble active agent used to practice embodiments as provided herein, or a fat-solubilized water-soluble active agent to act with a bivalent or trivalent metal salt.

In one embodiment, solid lipid suspensions can be used to formulate and to deliver compositions used to practice embodiments as provided herein to mammalian cells in vivo, in vitro or ex vivo, as described, for example, in U.S. Pat. Pub. No. 20050136121.

### Delivery Vehicles

In alternative embodiments, any delivery vehicle can be used to practice the methods as provided herein, for example, to deliver compounds and compositions as provided herein, or a compound used to practice methods as provided herein, to mammalian cells, for example, in vivo, in vitro or ex vivo. For example, delivery vehicles comprising polycations, cationic polymers and/or cationic peptides, such as polyethyleneimine derivatives, can be used for example as described, for example, in U.S. Pat. Pub. No. 20060083737.

In one embodiment, a dried polypeptide-surfactant complex is used to formulate compounds and compositions as provided herein, or a compound used to practice embodiments as provided herein, for example as described, for example, in U.S. Pat. Pub. No. 20040151766.

In one embodiment, compounds and compositions as provided herein, or a compound used to practice methods as provided herein, can be applied to cells using vehicles with cell membrane-permeant peptide conjugates, for example, as described in U.S. Pat. Nos. 7,306,783; 6,589,503. In one aspect, the composition to be delivered is conjugated to a cell membrane-permeant peptide. In one embodiment, the composition to be delivered and/or the delivery vehicle are conjugated to a transport-mediating peptide, for example, as described in U.S. Pat. No. 5,846,743, describing transport-mediating peptides that are highly basic and bind to poly-phosphoinositides.

In one embodiment, electro-permeabilization is used as a primary or adjunctive means to deliver the composition to a cell, for example, using any electroporation system as described for example in U.S. Pat. Nos. 7,109,034; 6,261,815; 5,874,268.

### Dosaging

The pharmaceutical compositions and formulations as provided herein or as used in methods as provided herein can be administered for prophylactic and/or therapeutic treatments. In therapeutic applications, compositions are administered to a subject, for example, a human in need thereof, in an amount of the agent sufficient to cure, alleviate or partially arrest the clinical manifestations and/or its complications (a “therapeutically effective amount”).

The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. Dosage levels may range from about 0.01 mg per kilogram to about 100 mg per kilogram of body weight. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, for example, Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods as provided herein are correct and appropriate.

### CRISPR-Based Therapy

In alternative embodiments, provided are methods for treating or ameliorating a cancer in an individual in need thereof comprising administering to the individual in need thereof an inhibitor of a squalene synthase or an Fdft1 inhibitor, wherein optionally the inhibitor of a squalene synthase or Fdft1 is a small molecule, a peptide or a polypeptide, or a nucleic acid inhibitor of squalene synthase or Fdft1 expression or activity, and optionally the nucleic acid inhibitor of squalene synthase or Fdft1 inhibitor expression or activity comprises an antisense or miRNA molecule, and/or a CRISPR cassette or nucleic acid module.

In alternative embodiments, the nucleic acid inhibitor comprises use of a CRISPR system in vivo or ex vivo, or use of a CRISPR-Cas9 system.

Delivery of CRISPR cassettes or nucleic acid modules as exogenous nucleic acids include delivery of Cas9, sgRNA, and associated complexes into cells such as T cells, and this delivery can occur using viral and non-viral systems: for example, electroporation of DNA, RNA, or ribonucleocomplexes; chemical transfection techniques utilizing lipids and peptides (particularly to introduce sgRNAs in complex with Cas9 into cells); nanoparticle-based delivery for transfection, and the like. Some categories of cells are more difficult to transfect, including stem cells, neurons, and hematopoietic cells, and these require more efficient delivery systems, such as those based on lentivirus (LVs), adenovirus (AdV), and adeno-associated virus (AAV).

Variants of CRISPR-Cas9 an be used to allow gene activation or genome editing with an external trigger such as light or small molecules: including photoactivatable CRISPR systems developed by fusing light-responsive protein partners with an activator domain and a dCas9 for gene activation, or by fusing similar light-responsive domains with two constructs of split-Cas9, or by incorporating caged unnatural amino acids into Cas9, or by modifying the guide RNAs with photocleavable complements for genome editing.

In alternative embodiments, “dead” versions of Cas9 (dCas9) are used to eliminate CRISPR's DNA-cutting ability while preserving its ability to target desirable sequences. Various regulatory factors can be added to dCas9s, enabling turning any gene on or off or adjust its level of activity. Like RNAi, CRISPR interference (CRISPRi) can turn off genes in a reversible fashion by targeting, but not cutting a site. The targeted site is methylated, epigenetically modifying the gene. This modification inhibits transcription. These precisely placed modifications may then be used to regulate the effects on gene expression (for example, HMGB2) and DNA dynamics after the inhibition of certain genome sequences within DNA.

In alternative embodiments, CRISPR-Cas13 fused to deaminases is used to direct mRNA editing; for example, Cas7-11, is better suited for therapeutic RNA editing than Cas13, and enables sufficiently targeted cuts.

In alternative embodiments, any CRISPR system can be used to practice methods as provided herein, for example, as described in US 2022 0387560 A1, which describes methods of treating and/or correcting ocular disease in vivo using an Adeno-associated virus (AAV) system, where the AAV system employs a nucleic acid encoding a CRISPR-Cas9 system for targeted gene disruption or correction; or US 2022 0389398 A1 that describes using engineered CRISPR/Cas effector enzymes, such as Cas13 (Cas13d, Cas13e, or Cas13f) that maintain guide-sequence-specific endonuclease activity and lack guide-sequence-independent collateral endonuclease activity; or US 2023 0029506 A1, which describes therapeutic applications of the crispr-cas systems and compositions for genome editing; or, US 2020 0340012 A1, which describes a modular CRISPR-Cas9 architecture that allows better delivery, specificity and selectivity of gene editing; or U.S. Pat. No. 8,771,945, which describes CRISPR-Cas systems and methods for altering expression of gene products; or WO 2023 283420 A2 which describes therapeutic gene silencing with crispr-cas13.

### Products of Manufacture and Kits

Provided are products of manufacture and kits for practicing methods as provided herein, which can optionally comprise recombinantly or genetically engineered cells such as CAR-T cells or genetically engineered CD8 T cells treated in vitro/ex vivo with an inhibitor of squalene synthase (or Fdft1). In alternative embodiments, products of manufacture and kits as provided herein further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, 15 “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Metabolic Programs of T Cell Tissue Residency Empower Tumor Immunity

This example demonstrates that methods and compositions as provided herein using exemplary embodiments are effective for metabolically enhancing chimeric antigen receptor (CAR)-T cells, including genetically engineered CD8 T cells, which target cancer cells, thereby enhancing cell function and persistence.

To systematically define the basis for the metabolic reprogramming supporting T_{RM} differentiation, survival, and function, we leveraged in vivo functional genomics, untargeted metabolomics, and transcriptomics of virus-specific memory CD8 T cell populations. We found that memory CD8 T cells deployed a range of adaptations to tissue residency, including reliance on non-steroidal products of the mevalonate/cholesterol pathway, such as Coenzyme Q (CoQ), driven by increased activity of the transcription factor Srebp2. This metabolic adaptation was most pronounced in the small intestine (SI), where T_{RM} interface with dietary cholesterol and maintain a heightened state of activation⁴, and was shared by functional TIL in diverse tumor types in mice and humans. Enforcing CoQ synthesis through Fdft1 deletion or Pdss2 overexpression promoted mitochondrial respiration, memory formation upon viral infection, and enhanced antitumor immunity. In sum, through a systematic exploration of T_{RM} metabolism, we reveal how these programs can be leveraged to fuel CD8 T cell memory formation in the context of acute infections and enhance antitumor immunity.

To maximize immune protection, memory CD8 T cells differentiate into functionally distinct populations with different surveillance capacities. These include effector memory T cells (T_{EM}) that mostly recirculate through tissues, central memory T cells (T_{CM}) that act as sentinels in lymphoid organs, and T_{RM}, which become a permanent asset to tissues throughout the body¹. T_{RM} constitute the majority of the CD8 T cell memory pool⁵, can retain the potential for supernumerary division and longevity^{6,7}, and offer a rapid and potent first line of defense due to their proximity to sites of reinfection and enhanced cytotoxic potential⁸⁻¹³, mediating protection against viral and bacterial infections^{8-11,14-17}. Similarly in tumors, pre-existing T_{RM} as well as TIL with T_{RM} features (T_{RM}-like) play a crucial role in suppressing the emergence of malignancies and mediating tumor control upon immune checkpoint blockade due to their superior intrinsic functional activity^{3,17-21}. The different environments in which these memory CD8 T cell subsets reside condition their function and differentiation²² potentially imposing distinct metabolic adaptations that support cell fitness. In addition, cellular metabolism can steer the course of CD8 T cell differentiation by linking specific enzymes and metabolites to cell fates and functions^{22,23}. Thus, an understanding of such mechanisms offers actionable strategies to program T cell subtypes that augment protection against infection and enhance current immunotherapies. While the major transcriptional programs that guide T_{RM} differentiation are established^{4,17,24-27}, a systematic investigation of the metabolic changes that allow T_{RM} formation and function is lacking.

### a Systematic Profiling of T_{RM} Metabolism

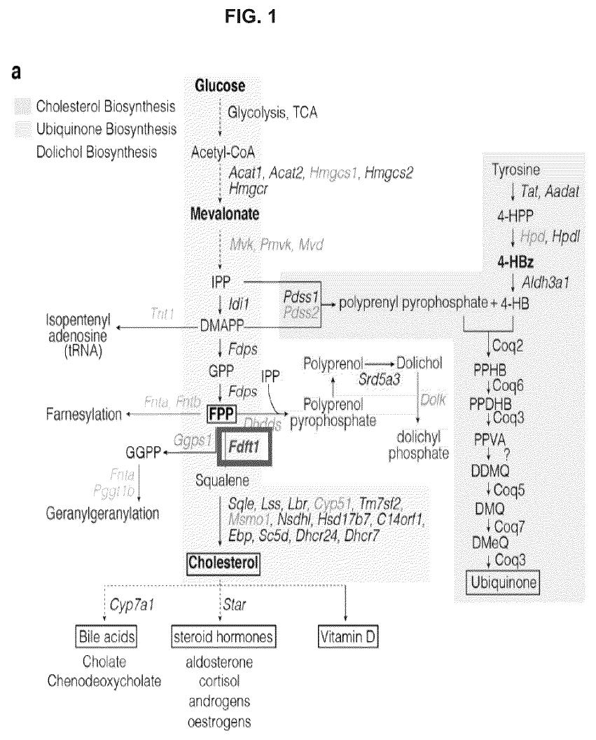

The transcriptional and functional inter-tissue heterogeneity of T_{RM}^{24,27} indicate that metabolic adaptions to diverse tissue environments accompany their differentiation. Indeed, we observe activation-dependent and tissue-specific gene-expression changes of metabolic signatures by different lymphocytic choriomeningitis virus (LCMV)-specific CD8 T cells subsets (Extended Data FIG. 1a). To uncover the functional metabolic regulators of T_{RM} formation, we performed a pooled CRISPR/Cas9-mediated loss-of-function screen targeting 3017 metabolic genes in the context of LCMV infection in vivo (FIG. 1a). We utilized P14 T cell receptor transgenic CD8 T cells, specific for the LCMV glycoprotein 33-41 peptide (GP₃₃₋₄₁) presented by H2-D^{b}, that constitutively expressed Cas9, allowing gene-editing by CRISPR/Cas9 (P14 Cas9eGFP). P14 Cas9eGFP cells transduced with the metabolic regulator targeting sgRNA library were adoptively transferred into WT recipients that were then infected with LCMV-Armstrong. Two weeks after infection, sgRNA-transduced P14 Cas9eGFP T cells were recovered, including circulating memory populations T_{EM}, T_{CM}, and t-T_{EM} from the spleen; small intestine (SI) T_{RM}, and liver T_{RM} and T_{EM}, allowing comparison of circulating to tissue-resident populations as well as comparison of two different tissue-resident populations. At this time point, P14 Cas9eGFP cells have seeded peripheral tissues and show a T_{RM} phenotype while allowing sufficient cell recovery¹⁷ (FIG. 1a). Intravenous (IV) labeling of CD8 T cells before cell isolation allowed discrimination of tissue-localized T_{RM} (IV⁻) from cells in circulation⁵. Consistent with a process of in vivo selection, sgRNA frequencies were more heterogenous in the recovered memory populations than in the input populations (Extended Data FIG. 1b). To assess the impact of each gene on memory T cell subsets, individual sgRNA abundance for each sample was compared to the initial input to calculate Log₂ Fold Change (Log₂ FC) enrichment and pooled to obtain gene-level scores. We focused first on the 155 metabolic genes that were statistically significant in their impact on SI T_{RM} formation (FIG. 1b and Supplementary Table 1). Most gene disruptions showed detrimental effects on memory CD8 T cell formation irrespective of subset or tissue (FIG. 1b). Unsupervised clustering grouped SI and liver T_{RM} together, suggesting that, despite clear differences between these two tissues, T_{RM} were metabolically different from circulatory populations (FIG. 1b). Further, a subset of genes was uniquely depleted in SI T_{RM}, including Cardiolipin synthase 1 (Crls1), a metabolic regulator known to support the mitochondrial function of SI T_{RM}⁴ (FIG. 1b). Finally, 6 genes led to increased SI T_{RM} formation when disrupted (FIG. 1b and Extended Data FIG. 1c). These genes included known negative regulators of CD8 T cell function and proliferation, such as Regnase-1 (Zc3h12a)²⁸, Pten²⁹, and Inpp5d³⁰. To gain a higher-level view of the processes represented by these individual gene scores, we classified each target by metabolic pathway (Supplementary Table 2). Among the positive metabolic regulators of SI T_{RM}, the “Oxidative phosphorylation complex III (OXPHOS Complex III)” and the “mevalonate/cholesterol synthesis” pathways were highly enriched in SI T_{RM} (FIG. 1c and Supplementary Table 3).

Next, we determined the relative abundance of intracellular metabolites in ex vivo populations of T_{RM} from the SI and kidney, and T_{EM}, T_{CM} and t-T_{EM} from the spleen by untargeted metabolomics (Extended Data FIG. 1d). Principal component analysis (PCA) of metabolite composition of these populations revealed a separation among T_{RM} and circulatory memory CD8 T cell fractions along PC1, and separation between kidney and SI T_{RM} along PC2 (FIG. 1d). In agreement with our transcriptional and functional data, these data suggested that T_{RM} in general have a distinct metabolic profile compared to their circulatory counterparts, as well as unique profiles associated with specific tissues. Unsupervised clustering based on identified, annotated metabolites revealed a similar sample aggregation and highlighted key intermediaries of the mevalonate/cholesterol synthesis pathway, mevalonate and mevalonolactone (loss-of-water adduct of mevalonate), as elevated in T_{RM}, and SI T_{RM}, respectively (FIG. 1e, Extended Data FIG. 1e, f, and Supplementary Table 4). Thus, data from our functional screen as well as gene expression and metabolomics profiling each revealed increased mevalonate/cholesterol synthesis as a potential metabolic adaptation of SI T_{RM}.

Rapid generation of mevalonate-derived products, including cholesterol, is required for initial rapid CD8 T cell proliferation after TCR activation³¹. Consistent with this, the transcriptional upregulation of enzymes of this pathway peaked early after LCMV infection, followed by a decline as CD8 T cells differentiate into memory CD8 T cell populations, with the notable exception of SI T_{RM}, where expression remained elevated well after resolution of infection (FIG. 1f and Extended Data FIG. 2a, b). Sustained expression of the mevalonate/cholesterol synthesis pathway was not unique to SI T_{RM}, as T_{RM} from other tissues maintained enrichment of this pathway compared to circulating memory CD8 T cell populations in the spleen and blood, however to a lesser degree (FIG. 1g and Extended Data FIG. 2c,d). Multiple data sets of mouse and human CD8 T cells showed similar enrichment patterns for expression of genes in the mevalonate/cholesterol synthesis pathway (Extended Data FIG. 2a-f).

Expression of the enzymes of the mevalonate/cholesterol synthesis pathway are collectively enhanced by the transcription factor (TF) sterol regulatory element binding protein 2 (Srebp2, encoded by Srebf2). Bioinformatics analysis by Taiji³², which leverages chromatin accessibility data combined with RNA expression, predicted Srebp2 as an upstream regulator of gene expression in SI T_{RM}²⁴ (FIG. 1h). Srebp2 protein was more abundant in T_{RM} of the SI compared to those of the kidney, and CD8 T cells in the spleen (FIG. 1i). Similarly, protein levels of HMG-CoA reductase (HMGCR), an Srebp2 target, and the rate-limiting enzyme of the mevalonate/cholesterol synthesis pathway, were also higher in human SI and colonic CD8 T cells compared to those in the spleen (Extended Data FIG. 2g). Runx3, necessary for the programming of CD8 T cell tissue residency¹⁷, also regulated expression of mevalonate/cholesterol pathway enzymes by CD8 T cells (Extended Data FIG. 2h). Thus, T_{RM} possessed a distinct metabolic program compared to their circulatory counterparts, including adaptations more prevalent in certain tissues, such as enrichment of the mevalonate/cholesterol synthesis pathway driven by Srebp2, which was most active in SI T_{RM}.

### Srebp2 Promotes T_{RM} Formation

To functionally assess the contribution of Srebp2 to T_{RM} formation, we adoptively transferred a 1:1 mix of P14 T cells transduced with control Cd19 shRNAmir- or Srebf2 shRNAmir-encoding retroviruses into mice that were subsequently infected with LCMV (FIG. 2a). Srebf2 shRNAmir suppressed Srebf2 expression as well as its downstream target Hmgcr by 5-10-fold (Extended Data FIG. 3a), and impaired T_{RM} formation in the SI with minimal impact on T cell accumulation in kidney, WAT, or liver (FIG. 2a). CD8 T cell populations in the spleen were unaffected by Srebf2 KD at both effector and memory time points (Extended Data FIG. 3b,c). However, full deletion of Srebp2 (P14 CD8 T cells transduced with control (sgCd19) or Srebf2 sgRNA-encoding retroviruses (sgSrebf2)), impaired T_{RM} formation in SI, kidney and WAT, but spared circulatory memory CD8 T cell populations despite decreased effector T cell accumulation at day 7 (FIG. 2b and Extended Data FIG. 3d-f). Liver T_{RM} showed less dependence on Srebp2; IV⁻ P14 CD8 T cells were modestly impaired and when defined as CD69⁺CD62L⁻, an alternative gating strategy³³, liver T_{RM} were unaffected by loss of Srebp2 (FIG. 2b). SI T_{RM} populations induced by Listeria monocytogenes (LM) infection also required Srebp2, suggesting the reliance on this pathway in infection was generalizable (FIG. 2c). Together, these results showed that sustained Srebf2 expression was functionally relevant for the formation of T_{RM} but not for circulating memory T cell populations.

The inactive form of Srebp2 in the endoplasmic reticulum (ER) becomes activated under conditions of low ER cholesterol by its translocation to the Golgi by Srebp activating-cleavage protein (Scap)³⁴, and thus loss of Scap impairs Srebp2 activation³⁴. To understand the implications of mevalonate/cholesterol synthesis inhibition in polyclonal endogenous CD8 T cell responses, we infected mice with a T cell-specific deletion of Scap (Scap^{fl/fl} Cd4-Cre⁺, Scap KO) and followed the LCMV-specific CD8 T cell response of H-2Db-GP₃₃₋₄₁ tetramer (Tet⁺) compared to WT (Scap^{wt} Cd4-Cre⁺) mice. As previously shown³³, Tet⁺ Scap knock-out (KO) cells displayed reduced accumulation during the effector phase compared to WT Tet⁺ cells³¹ (FIG. 2d and Extended Data FIG. 3g). Scap KO showed impaired differentiation of Tet⁺ CD103⁺CD69⁺ SI T_{RM}, but not kidney or liver T_{RM} (FIG. 2d, Extended Data FIG. 3h). Concomitant with impaired T_{RM} accumulation, Scap KO mice that had been infected with LM-GP₃₃₋₄₁ had higher viral titers in the SI when rechallenged with LCMV compared to WT mice (FIG. 2e). While loss of Scap reduces fatty acid synthesis, as it also controls Srebp1 (Srebf1) activity, neither Srebf1 nor Fatty Acid Synthase (Fasn) impacted memory T cell differentiation (Supplementary Table 1).

HMG-CoA reductase (HMGCR) inhibitors (statins) are widely consumed lipid-lowering drugs with known immunomodulatory effects³⁵. We found that lovastatin treatment impaired the accumulation of P14 CD8 T cells responding to LCMV in the SI compared to the control group on day 7 of infection; by day 21 there was a sustained loss of SI T_{RM} and impaired accumulation of memory T cells in the spleen as well (FIG. 2f). These observations are of potential human significance as we observed lower expression of the T_{RM} gene-expression signature¹⁷ in SI CD8 T cells from statin users than non-statin users, revealing a potentially underappreciated effect of statins on T_{RM} populations of the SI (FIG. 2g and Supplementary Table 5).

Each of these conditions inhibit the mevalonate/cholesterol synthesis pathway to varying degrees. Combined, these data suggested that enhanced activity downstream of Hmgcr, Scap, and Srebp2 is a metabolic requirement of CD8 T cells to become SI T_{RM}. We observed graded reliance on Srebp2 among the different tissues; where SI T_{RM} showed a greater dependence on the pathway than kidney, WAT, and liver resident cells, reflecting the pattern of expression for genes involved in this pathway (FIG. 1g).

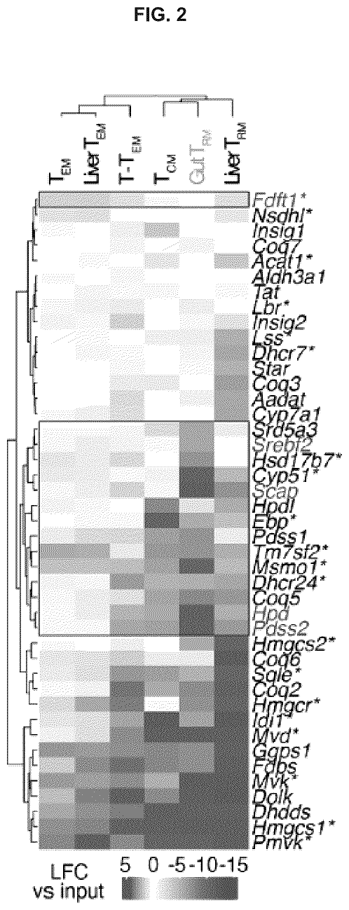

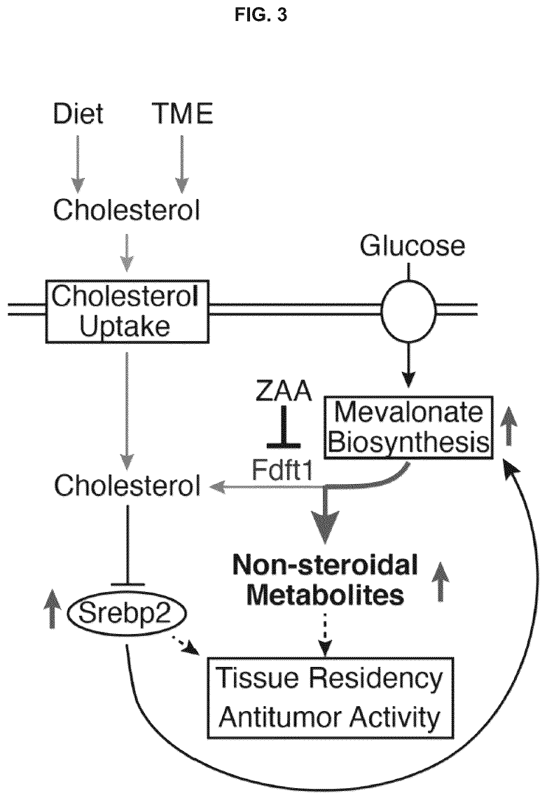

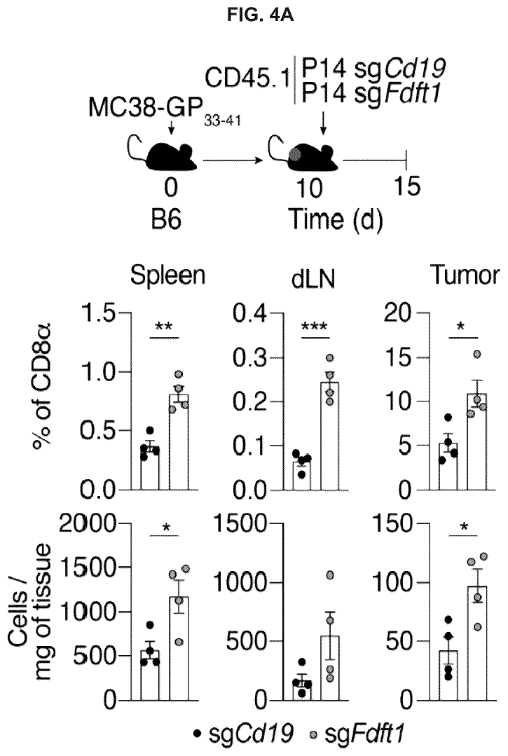

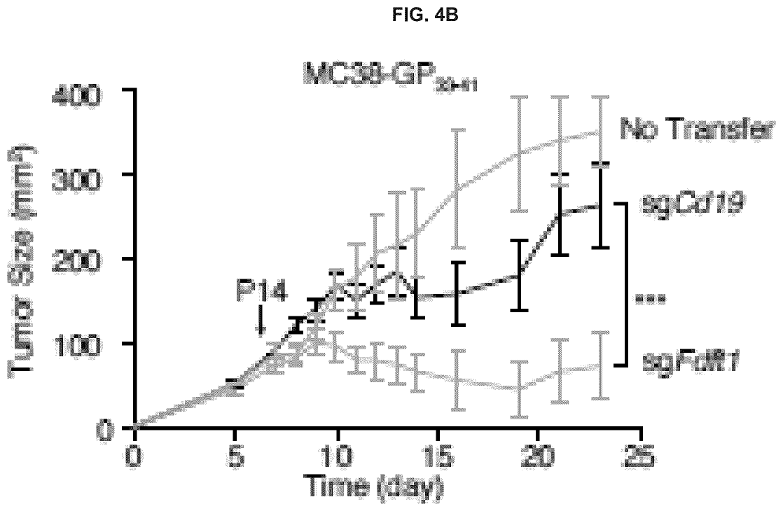

Interestingly, enforcing Srebp2 activity by ectopic overexpression of a constitutively active nuclear isoform actually prevented P14 CD8 T cell accumulation in the SI after LCMV infection, despite successfully increasing total cellular cholesterol and its downstream target, low density lipoprotein receptor (LDLR) (Extended Data FIG. 3i, j). This additional observation is consistent with the idea that generating high cholesterol levels may not be the rate-limiting step for T_{RM} populations. To further investigate the impact of Srebp2 loss, we compared gene expression by shCd19- or shSrebf2-transduced P14 CD8 T cells after LCMV infection. Srebf2 and the mevalonate/cholesterol synthesis pathway-associated enzymes were most highly expressed in SI T_{RM} and reduced in the shSrebf2-transduced P14 CD8 T cells (Extended Data FIG. 4a-c), and a core T_{RM} gene-expression signature¹⁷ was downregulated in shSrebf2 P14 CD8 T cells in the SI (Extended Data FIG. 4d-e). Strikingly, few genes were differentially expressed beyond Srebp2 and Srebp2-dependent genes (Extended Data FIG. 4f), suggesting that impaired SI T_{RM} accumulation upon Srebp2 knockdown was not likely the result of transcriptional changes beyond the mevalonate/cholesterol synthesis pathway.

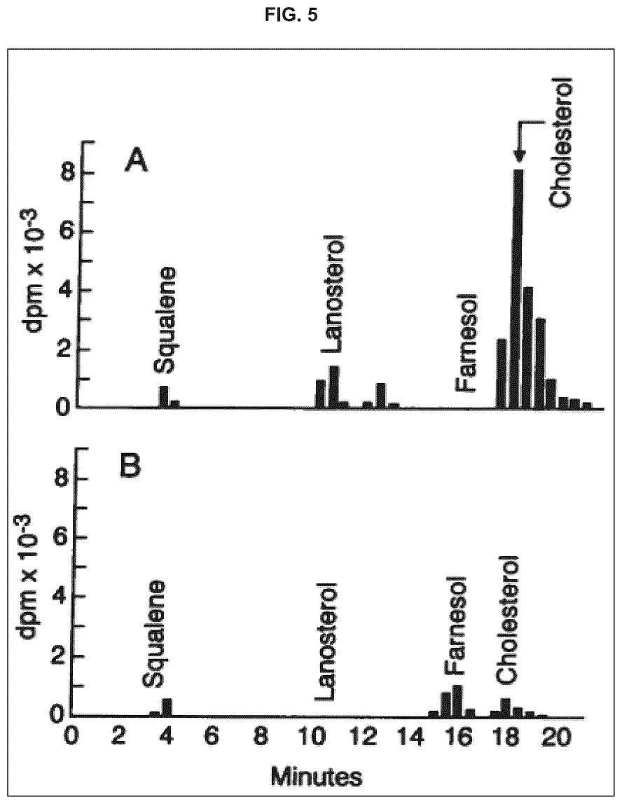

SI T_{RM} are unique in their close contact with food-derived nutrients in the absorptive lining of the intestine³⁶. Given that Srebp2 activity is regulated by the strong inhibitory feedback exerted by cholesterol (present in the diet), we sought to understand how the intestinal environment affected Srebp2-dependent metabolic programs on SI T_{RM}. Notably, memory P14 CD8 T cells in the SI, but not the spleen, showed significant uptake of orally administered cholesterol (Extended Data FIG. 5a). On the other hand, when administered IV, none of the memory CD8 T cell populations profiled, including SI T_{RM}, showed significant in vivo uptake of cholesterol-containing low-density lipoproteins (LDL), the main form of systemic delivery of cholesterol to cells (Extended Data FIG. 5b). Using imaging flow cytometry, we found that SI T_{RM} of mice fed regular chow had the lowest amount of intracellular cholesterol compared to the kidney, liver, and splenic population (Extended Data FIG. 5c), consistent with maintained Srebp2 activity. Feeding a high cholesterol-containing diet partially reduced Srebp2-dependent genes, Hmgcr, Hmgcs1, and Ldlr in CD8 T cells of the SI, with minimal effects in splenic CD8 T cells (Extended Data FIG. 5d) and impaired SI T_{RM}, but not kidney or liver T_{RM}, formation and maintenance (FIG. 2h and Extended Data FIG. 5e,f). Of note, the diet high in cholesterol did not impact food intake or mouse weight, and only transiently raised circulating cholesterol levels compared to the low cholesterol diet control (Extended Data FIG. 5g-i). Ultra-low input (ULI)-RNAseq analysis of SI T_{RM} from mice fed a high cholesterol diet showed marked suppression of Srebp2 targets, as well as induction of programmed cell death-related genes and cholesterol efflux transporters, compared SI T_{RM} from mice fed a low cholesterol diet (Extended Data FIG. 5j-4 and Supplementary Table 6). Thus, SI T_{RM} take up and adapt to differences in dietary cholesterol, but when in excess, either by enforcing Srebp2 OE (Extended Data FIG. 3i) or over-feeding (FIG. 2h), cholesterol was detrimental to SI T_{RM} accumulation. While increasing cholesterol in the diet attenuated Srebp2 activity, it was insufficient to reduce it to levels observed in the spleen (Extended Data FIG. 5d) suggesting additional cues, beyond environmental cholesterol, likely maintain Srebp2 activity in this environment, such as RUNX3™ (Extended Data FIG. 2g). Further, these data suggested that, rather than cholesterol, alternative products of the mevalonate/cholesterol synthesis pathway are the relevant mediators of T_{RM} formation downstream of Srepb2.

### CoQ-Producing Enzymes Regulate T Cell Fates

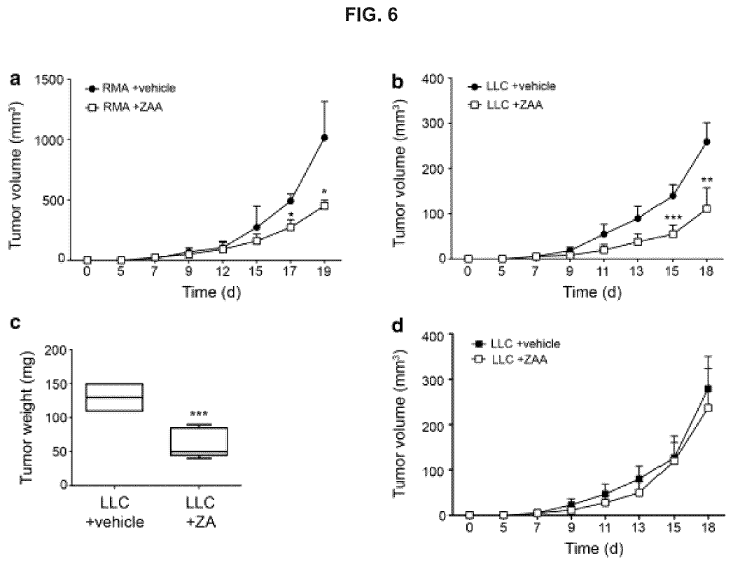

Intermediate products of the mevalonate/cholesterol synthesis pathway supply substrates to produce important nonsterol isoprenoids, such as CoQ, through branched reactions. To explore the mechanistic basis for T_{RM} dependence encompassing these alternative fates of mevalonate, we focused on additional enzymes that regulate diversion of intermediates to synthesis of non-steroidal metabolites from the in vivo CRISPR/Cas9 (extended mevalonate/cholesterol synthesis pathway) (FIG. 3a and Extended Data FIG. 6a). Clustering of these genes by their relative impact on CD8 T cell memory populations revealed a group that were more specific to SI T_{RM} (FIG. 3a). Among these genes, Pdss2 and Hpd, two enzymes that divert metabolites to the synthesis of CoQ, were both particularly required for SI T_{RM}, and targeting led to similar loss of T_{RM} phenotypes as disruption of Srebf2 and Scap (FIG. 3a). Notably, our metabolomics analysis found 4-hydroxybenzaldehyde (4-Hb), the product of Hpd enzyme, to be especially elevated in SI T_{RM} compared to other CD8 T cell populations, indicating this pathway might be increased in SI T_{RM} (Extended Data FIG. 6b,c). Validation of the CRISPR/Cas9 screen by targeting Hpd revealed impaired formation of SI T_{RM}, but not P14 memory T cells in the spleen, liver, or kidney (FIG. 3b and Extended Data FIG. 6d). These results suggested SI T_{RM} have a heightened requirement for CoQ synthesis compared to other memory CD8 T cell populations. Further, deletion of squalene synthase (Fdft1), which catalyzes the first step of the isoprenoid branch committed specifically to cholesterol production, appeared to promote several populations of memory CD8 T cells (FIG. 3a). Consistent with the CRISPR/Cas9 screen, targeting Fdft1 resulted in increased accumulation of liver T_{RM}, as well as circulating memory subsets, but it was not sufficient to further enhance SI T_{RM} (FIG. 3c and Extended Data FIG. 6e). These data reinforce the notion that the production of cholesterol by the mevalonate pathway is not required and revealed a metabolic enzyme, Fdft1, to be a potentially actionable target to improve memory CD8 T cell formation.

To gain greater insight into the identity and mechanism of action of the relevant non-steroidal products mediating greater memory potential to Fdft1 KO CD8 T cells, we conducted a secondary targeted in vivo CRISPR/Cas9 screen of potential non-steroidal mediators of Fdft1 impact on T cell accumulation. This library, composed of 153 sgRNAs targeting 50 genes of the extended mevalonate/cholesterol synthesis pathway (Extended Data FIG. 6a and Supplementary Table 7), was transduced into either control (sgCd19) or sgFdft1 P14 Cas9eGFP T cells to test their function in mediating Fdft1-enhanced T_{EM} and liver T_{RM} populations (FIG. 3c,d). By plotting their relative impact, we looked for genes that had a significant effect in reducing the accumulation of sgFdft1 while sparing control sgCd19 P14 T cells in memory populations, as these likely mediate the enhanced accumulation observed (FIG. 3d, Extended Data FIG. 6f, and Supplementary Table 8). Comparison to T_{E} and T_{MP} cells from day 7 of infection provided a reference for identifying genes that were required only at the memory timepoint rather than at the effector stage, such as Ggps1 or Trit1. Genes that affected both populations equally, and thus fall in the plot's diagonal, do not likely mediate the specific advantage observed upon Fdft1 deletion (FIG. 3d). This analysis revealed that several enzymes needed for CoQ synthesis (Coq2), geranylgeranylation (Ggps1) and tRNA modification (Trit1) showed a larger effect on Fdft1 KO CD8 T cells than control CD8 T cells in spleen T_{EM}, of which only Coq2 was specific to memory cells (FIG. 3d). Further, the dependence on CoQ synthesis was more striking for the T_{RM} population, as it included additional enzymes, such as Hpd, Pdss1, and Pdss2 (FIG. 3d). Combined, these data revealed that the advantage endowed by Fdft1 loss is supported by the production of non-steroidal metabolites, including CoQ (Extended Data FIG. 6g).

Given the critical role of Pdss2 in our functional screens and its role in the first reaction producing CoQ from mevalonate products, we tested whether Pdss2 overexpression could mediate enhanced T_{RM} formation. Indeed, enforced Pdss2 expression led to a 3- to 8-fold increase in T cell accumulation compared to control vector across all tissues examined (FIG. 3e and Extended Data FIG. 6h). Of note, Pdss2 OE restored SI T_{RM} formation to WT levels in mice fed a high cholesterol diet (FIG. 3f). Combined, these data suggest that T_{RM} increase Srebp2 activity to meet a heightened requirement, particularly elevated in the SI, for non-steroidal products, such as CoQ. As such, promoting the accumulation of metabolic intermediates that can be diverted to CoQ production by blocking Fdft1, or boosting the capacity to shunt intermediates into the pathway by enforcing Pdss2 overexpression, enhances T_{RM} formation and can offset some of the toxicities induced by an excess of dietary cholesterol. Of note, circulating memory CD8 T cell populations similarly benefitted from Pdss2 OE (FIG. 3e), suggesting this pathway might generally increase T cell-mediated immunological memory.

### CoQ Supports T Cell Mitochondrial Respiration

CoQ is a highly hydrophobic redox lipid in the inner mitochondrial membrane that transports electrons from complexes I and II to complex III in the electron transport chain (ETC). We next tested whether CoQ production driven by the mevalonate/cholesterol synthesis pathway impacted mitochondrial respiration. Srebf2 KD CD8 T cells had lower rates of basal oxygen consumption (OCR) and reduced maximal respiration upon FCCP treatment (spare respiratory capacity, SRC) (FIG. 4a). Similarly, statin treatment reduced mitochondrial respiration of CD8 T cells in a mevalonate-dependent, but cholesterol-independent manner (FIG. 4b). A comparable loss of mitochondrial respiration was also observed in Pdss2-deficient CD8 T cells (FIG. 4c). Conversely, Fdft1 deletion increased basal OCR and SRC (FIG. 4d), in a Pdss2-dependent manner (FIG. 4e). Of note, Pdss2 OE was sufficient to promote higher basal and maximal mitochondrial respiration (FIG. 4f). In line with these data, deletion of Fdft1 or enforced expression of Pdss2, lead to higher production of CoQ species, including the most abundant form in mice, CoQ9, and its precursor DMQ9 (FIG. 4f,g). Taken together, these data suggest that elevated rates of mevalonate/cholesterol synthesis intermediaries supply the CoQ pool to potentiate mitochondrial respiration in CD8 T cells.

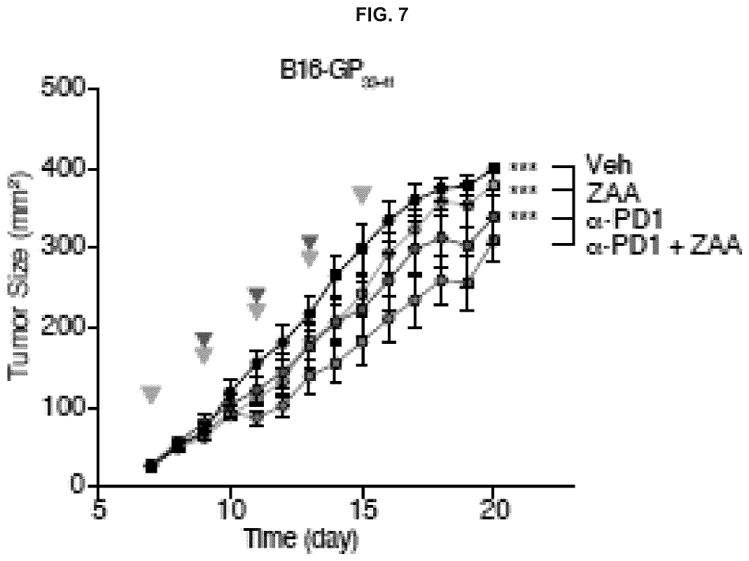

Given the relationship between T_{RM} and T_{RM}-like TIL, including the importance of mitochondrial respiration and transcriptional programming^{7,37,38}, we explored if common metabolic requirements might be shared between SI T_{RM} and functional TIL. To put our previous findings into the context of anti-tumor CD8 T cell responses, we performed scRNAseq profiling of P14 CD8 T cells responding to a murine tumor model (MC38-GP₃₃₋₄₁). The mevalonate/cholesterol synthesis pathway was elevated in P14 CD8 TIL versus those in the spleen (Extended Data FIG. 7a,b). Examining the different subpopulations revealed that the proliferating (C4), terminally exhausted (C3), and T_{RM}-like TIL (C7) expressed the highest levels of Srebp2-dependent targets, enzymes Hmgcs1 and Hmgcr (Extended Data FIG. 7c). Similarly, reanalysis of a human pan-tumor dataset revealed that the mevalonate/cholesterol pathway was most upregulated in proliferating, exhausted, effector, and T_{RM}-like populations (found mostly within the tumor) and low in memory and naive CD8 T cell populations (found mostly in circulation) across 21 different cancer types³⁹ (Extended Data FIG. 7d). Thus, in both human and mouse, increased expression of genes involved in mevalonate/cholesterol synthesis in TIL implicate this pathway in intratumoral T cell accumulation.

### The Srebp2-CoQ Axis Boosts Tumor Immunity

To functionally test whether T_{RM} and TIL shared common metabolic adaptations related to the mevalonate/cholesterol synthesis pathway and to pin down which of its components were most relevant, we performed two in vivo CRISPR/Cas9 screens in P14 CD8 T cells in parallel using the 50-gene library targeting key elements of this pathway (Supplementary Table 5): one in the context of LCMV infection for the formation of SI T_{RM}, and the second in the context of MC38-GP₃₃₋₄₁ tumors for the accumulation of TIL (FIG. 5a). This experiment revealed a significant correlation between the impact of these genes on TIL and SI T_{RM} accumulation, with 19 significant genes commonly required for both cellular populations (67% overlap) (FIG. 5b,c, Extended Data FIG. 7e and Supplementary Table 9). Targeted genes were essential for T cells in the tumor and for T_{RM} but were largely dispensable for T cells in the draining lymph node (dLN) (FIG. 5b). Most of the 19 genes with a shared requirement by T_{RM} and TIL mediate the production of non-steroidal metabolites, including Hpd and Coq2 that lead to CoQ production (FIG. 5c,d). As we observed for SI T_{RM}, these data showed that synthesis of cholesterol was not required for TIL accumulation (Dhcr7 and Dhcr24 were not essential) (FIG. 3a and FIG. 5c). Consistent with our studies in the context of infection, Srebf2 deficiency impaired accumulation of P14 CD8 T cells in the tumor (FIG. 5e), loss of Pdss2 reversed the increased accumulation of Fdft1 KO P14 CD8 T cells in the tumor (FIG. 5f), and Pdss2 OE promoted an even higher accumulation of P14 CD8 T cells in the tumor than in the spleen (FIG. 5g). Taken together, these data showed that both T_{RM} and TIL populations rely on a sustained and elevated Srebp2-driven program to synthesize non-steroidal metabolites, such as CoQ, to support their accumulation.

We next tested if it was possible to enhance T cell activity in tumors by enforcing the T_{RM} metabolic state. Fdft1 deletion increased P14-dependent tumor control of MC38-GP₃₃₋₄₁ tumors, accompanied by improved P14 CD8 T cell persistence in tumors, dLN, and spleens (FIG. 5h,i). Similarly, Pdss2 OE enhanced tumor control in B16-GP₃₃₋₄₁ melanoma tumors (FIG. 5j), and promoted a higher frequency of T_{RM}-like TIL (FIG. 5k). Of note, we did not find changes in the expression of exhaustion-associated markers or differences in cytokine production upon ex vivo restimulation (Extended Data FIG. 7g,h,i,j). Finally, Fdft1 inhibition by administration of a competitive metabolite derived from fungi, Zaragozic Acid A (ZAA), reduced tumor growth and improved the survival of mice implanted with MC38-GP₃₃₋₄₁ tumors in a CD8-dependent manner (Extended Data FIG. 7k,l,m). These data are consistent with previous reports of the immune system-dependent effects on antitumor control of ZAA⁴⁰. When combined with anti-programmed cell death protein 1 (PD-1) treatment, ZAA led to greater control of B16-GP₃₃₋₄₁ melanoma tumors, further indicating that Fdft1 may be a valid treatment target (FIG. 5l). Taken together, this study revealed that an understanding of the metabolic regulators of T_{RM} populations can be leveraged to improve CD8 T cell immunity against infections and tumors, including avenues to increase the production of CoQ downstream of Srebp2 (FIG. 5m).

### Discussion

Combining functional screens of metabolic dependencies with transcriptomic and metabolomic profiling of circulating and resident subsets of memory CD8 T cells offered a systematic approach to identifying how metabolism impacts memory CD8 T cell formation in response to viral infections. In agreement with previous findings^{25,27}, our results support the emerging concept that T_{RM} formation requires distinct transcriptional and metabolic adaptations to different tissue environments. These data describing multiple potentially novel regulators of CD8 T cell memory differentiation have been made publicly available in a web-based application, Functional Immunometabolism and Transcriptomics Database (FITdb, fitdb.de).

Here, we chose to focus on SI T_{RM} because these cells display profound metabolic differences from their circulating counterparts, maintain a controlled basal state of heightened activation resembling effector cells⁴, and possess molecular features associated with superior CD8 T cell functionality against tumors^{17,26}. We found the Srebp2-dependent upregulation of the mevalonate/cholesterol synthesis pathway as the most distinguishable metabolic feature of SI T_{RM}. Mechanistically, Srebp2 sustained transcriptional activation of the mevalonate/cholesterol biosynthesis pathway to support the production of non-steroidal products, including CoQ, which promoted mitochondrial respiration in vitro and in vivo accumulation in infection and tumors. We also showed that SI T_{RM} respond to dietary cholesterol, which when in excess, was detrimental to their formation. Together, these adaptations might explain previous studies reporting an increase of mevalonate/cholesterol enzymes by T cell populations of the intestine^{4,41,42}, and offer new insights into how dietary interventions could be leveraged to improve cellular immunity in the gastrointestinal tract^{36,43}. Statins impact immune responses³⁵, including reducing lymphocyte concentrations of CoQ⁴⁴. Interestingly, our results highlight the dependency of SI T_{RM} on this pathway and implicate a potentially significant effect in humans. Overall, our study identifies both dietary and pharmacological approaches to modulate T cell responses in the intestine.

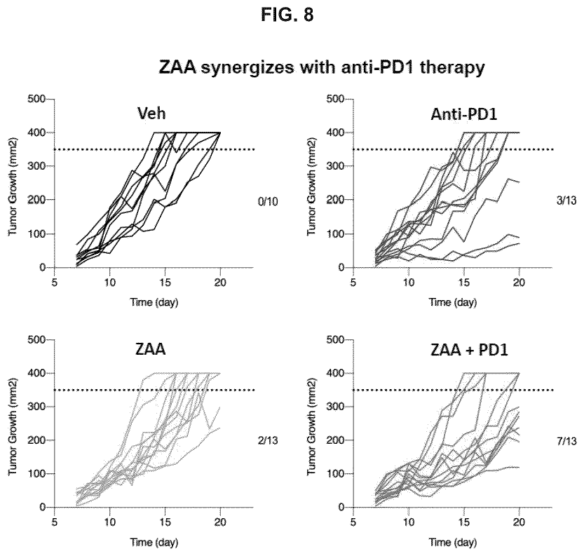

Our data further reveal a common dependency on mevalonate/cholesterol synthesis enzymes for SI T_{RM} formation and TIL accumulation. Based on these observations, we found two actionable enzymes, Fdft1 and Pdss2, which can be manipulated to enforce CoQ production, enhance the mitochondrial respiratory capacity of CD8 T cells, and promote in vivo accumulation of CD8 T cells in the context of infection and tumors. Whether these adaptations are conserved across all tumor types and contexts remains to be further investigated. The fact that loss of most genes of the mevalonate/cholesterol synthesis pathway impaired TIL accumulation in the melanoma model B16-OVA from a previous study²⁸ (Extended Data FIG. 8a), suggests these observations may be generalized to other tumor contexts.

T_{RM}-like TIL, and cells genetically endowed with superior antitumor properties, such as regnase 1 (an endoribonuclease) KO CD8 T cells²⁸, upregulate the mevalonate/cholesterol synthesis pathway in the absence of increased proliferation (Extended Data FIG. 8b). Based on our data, and given that cholesterol accumulation in the tumor microenvironment leads to intracellular cholesterol buildup in TIL conducive to dysfunction^{45,46}, we propose increased Srebp2-driven mevalonate/cholesterol pathway synthesis in TIL serves to generate non-steroidal metabolites, rather than cholesterol (FIG. 5m). Of note, deletion of genes leading to CoQ synthesis (ie Pdss2) were able to abolish the heightened antitumor potential of regnase-1 KO T cells (Extended Data FIG. 8c)²⁸. Certainly, the reliance on non-steroidal intermediates does not preclude the need for generating or obtaining sufficient cholesterol to support T cell functions during priming and activation in responses against pathogens and tumors. In sum, we provide conclusive evidence that T_{RM} utilize distinct metabolic adaptations, which can be exploited to enhance the function of CD8 T cells in solid tumors.

### Statistical Methods

Statistical tests were performed using Prism (7.0/8.0/9.0) (GRAPHPAD™). Two-tailed paired or un-paired t-Test was used to compare groups with normally distributed values unless otherwise specified. Mann-Whitney U test was performed when value distributions did not pass normality tests. A Two-sided one-sample t-Test was used to compare data distributions against a hypothetical value of 0. Two-way ANOVA was used to test the effects of two variables on a continuous outcome. Fisher's exact test was performed to determine the non-random association between two categorical variables. Logrank test was performed to test for differences between the populations in the probability of an event at any time point, related to survival analysis. P values of less than 0.05 were considered significant.

### Data and Materials Availability

Bulk RNA-seq, and single-cell RNA-seq have been deposited to GEO under the reference series GSE207044. In addition, the following published datasets were used: GSE107278¹⁷, GSE106107¹⁷, GSE182276²⁴, GSE70813⁴⁷, GSE157072⁴⁸, GSE125527⁴⁹, GSE131847⁴⁸, GSE137015²⁸, GSE70813⁴⁷, and GSE157072⁵⁰.

### FIGURE LEGENDS

FIG. 1, FIG. 2 and FIG. 3 schematically illustrate exemplary actionable metabolic interventions to enhance antitumor CD8 T Cell function.

FIG. 4A and FIG. 4B graphically illustrate data showing that loss of Fdft1 promotes TIL persistence and increased antitumor function. These data was generated by genetically manipulating P14 Cas9eGFP CD8 T cells with CRISPR from a mouse donor by retroviral transduction with an Fdft1 or an sgCd19-targeting sgRNA construct. After genetic manipulation and expansion in vitro, these cells were transferred into tumor-bearing mice. A group of control mice did not receive any modified P14 Cas9eGFP CD8 T cells. This experiment provides a proof of concept that inhibiting Fdft1 expression is a viable strategy to increase the antitumor potential of tumor-specific CD8 T cells in an adoptive cellular transfer setting.

FIG. 5 graphically illustrates data showing an increase in upstream derivates from FPP metabolism (Farnesols): dolichol; ubiquinone; geranylgeranylation; and, isoprenoid chains.

FIG. 6 graphically illustrates data showing that antitumor actions of zaragozic acid A (ZAA) depend on the immune system.

FIG. 7 graphically illustrates data showing that tumor Growth Curves in B16-F10 tumors implanted subcutaneously (s.c.) in B6 mice. Tumor-bearing mice were treated with a combination of Zaragozic Acid A and anti-PD1 antibody. This experiment shows that anti-PD1 therapy can be effectively and safely combined with Zaragozic Acid A to treat established tumors in mice.

FIG. 8 graphically illustrates data showing that ZAA synergizes with anti-PD1 therapy. Tumor-bearing mice were treated with a combination of Zaragozic Acid A and anti-PD1 antibody. This experiment shows that anti-PD1 therapy can be effectively and safely combined with Zaragozic Acid A to treat established tumors in mice. This experiment shows independent tumor growth curves from FIG. 7.

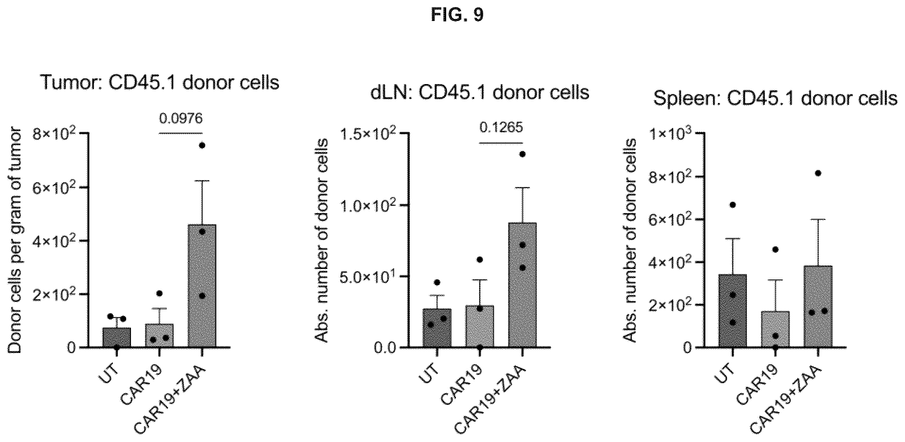

FIG. 9 graphically illustrates data showing that ZAA can be combined with CAR T cell treatment of established solid tumors to increase the ability of CAR T cells to accumulate intratumorally. Tumor-bearing mice received tumor-specific CAR T cells in combination with Zaragozic Acid A. Tumor cells contained the surface protein CD19, which is recognized by the CAR anti-CD19 on the CD8 T cell (CD19 CAR T cell).

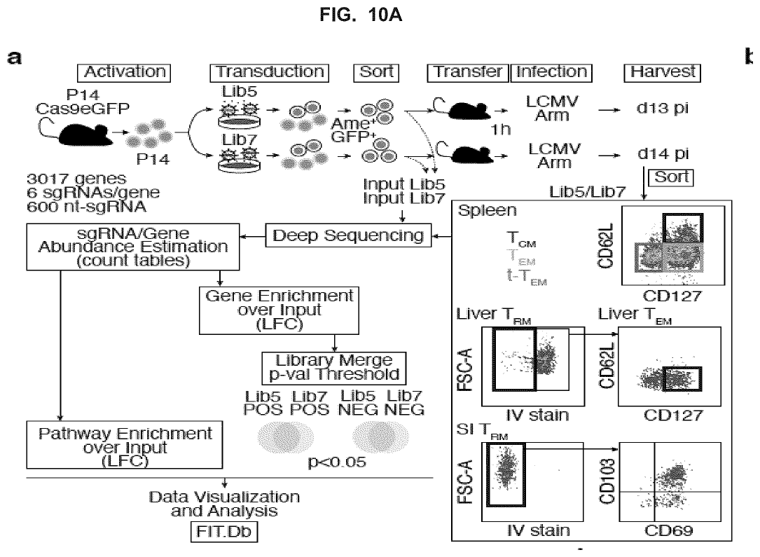

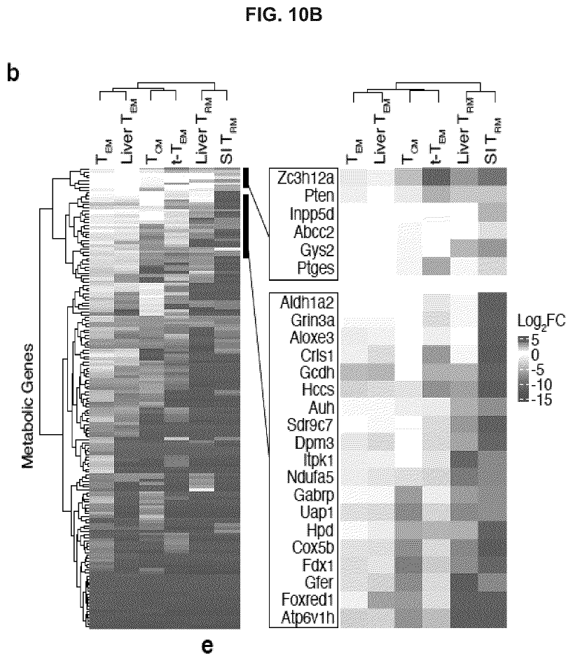

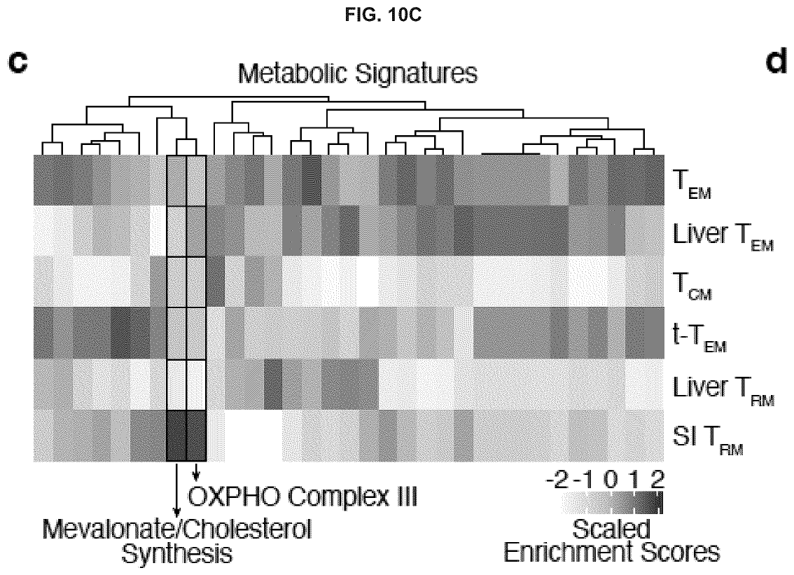

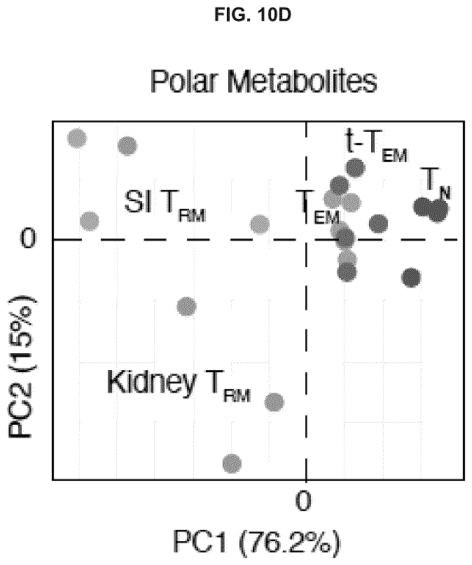

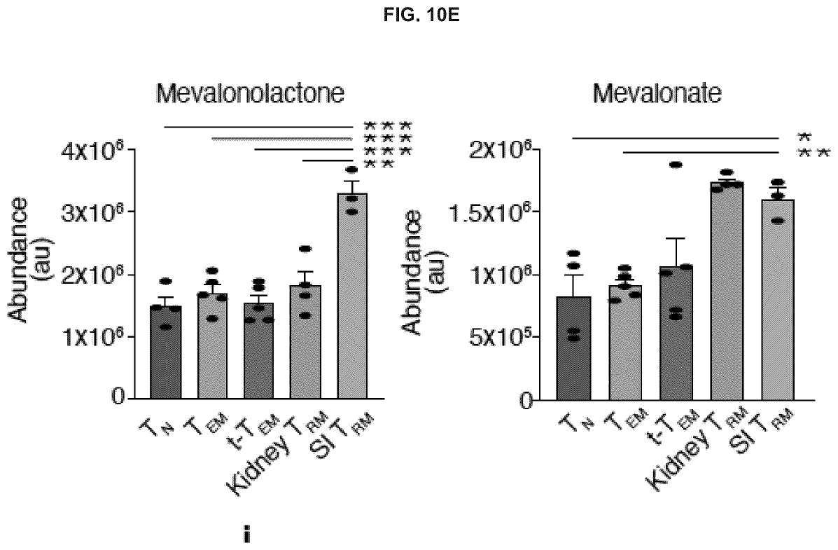

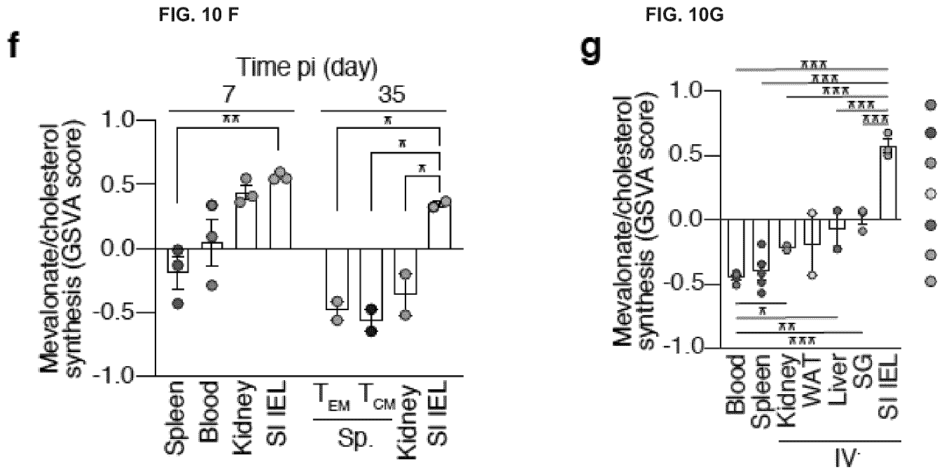

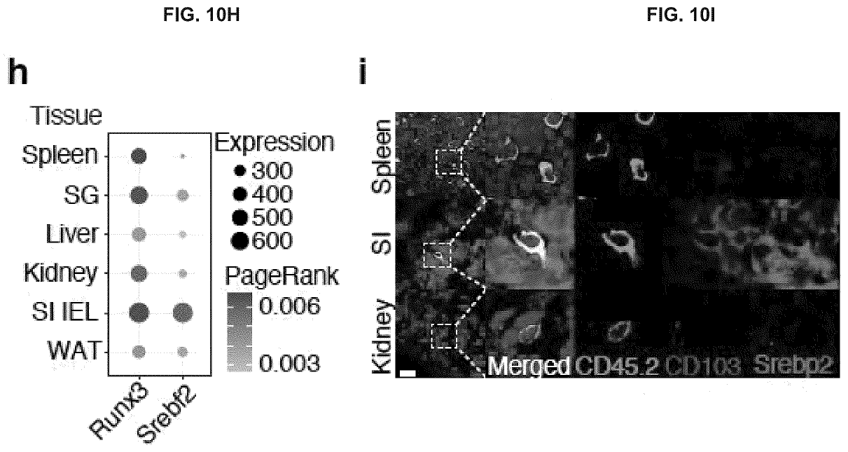

FIG. 10, or FIG. 1 of Example 1. Functional genetics in vivo, metabolomics and transcriptional analysis of ex vivo populations of memory CD8 T cells identify a graded upregulation of a Srebp2-dependent metabolic programming across T_{RM}.

a, Experimental design and data analysis approach for an in vivo CRISPR/Cas9-mediated loss-of-function screen of metabolic regulators of memory CD8 T cell differentiation in LCMV infection. b, Unsupervised hierarchical clustering heatmap of averaged gene essentiality scores for significant genes in SI T_{RM} in both libraries. c, Unsupervised hierarchical clustering of scaled averaged enrichment scores of significant metabolic signatures in SI T_{RM} in both libraries. d, PCA of relative metabolite abundances of indicated P14 CD8 T cells 13 days after LCMV infection. e, Abundance of selected identified annotated metabolites of mevalonate/cholesterol synthesis pathway on indicated P14 CD8 T cells. TN, Naïve. f, Mevalonate/cholesterol synthesis pathway GSVA scores from RNAseq analysis of indicated P14 CD8 T cells, GSE107278. g, Mevalonate/cholesterol synthesis pathway GSVA scores from RNAseq analysis of indicated memory P14 CD8 T cells (>30 days pi), GSE182276.

SG, salivary gland. WAT, white adipose tissue. h, PageRank scores and gene expression of Srebp2 and Runx3 in memory P14 CD8 T cells, GSE182276. i, Representative detection of Srebp2 and CD103 by immunofluorescence of congenically labeled memory P14 CD8 T cells. Scale bar, 20 μm. P14 CD8 T cells recovered from tissues other than the spleen were pregated on IV⁻ populations. Images are representative of 2 independent experiments (i). Data are mean+/−s.e.m., and representative of at least two independent experiments (f, g) with a total of n=2 (day 35) and n=3 (day 7) (f), n=2 (liver and WAT), n=3 (SG, kidney, and SI), n=4 (blood), and n=5 (spleen) (g) mice, or one experiment (a, b, c, d and e), with a total of n=7 (Lib7) and n=9 (Lib5) mice pooled per library (a, b, and c), and n=3 (SI), n=4 (TN and kidney), and n=5 (rest) (d and e) samples, where each sample contains cells pooled cells from 2 to 5 mice. Two-sided un-paired t-Test (e, f, and g). *P<0.05, **P<0.01, ***P<0.005.

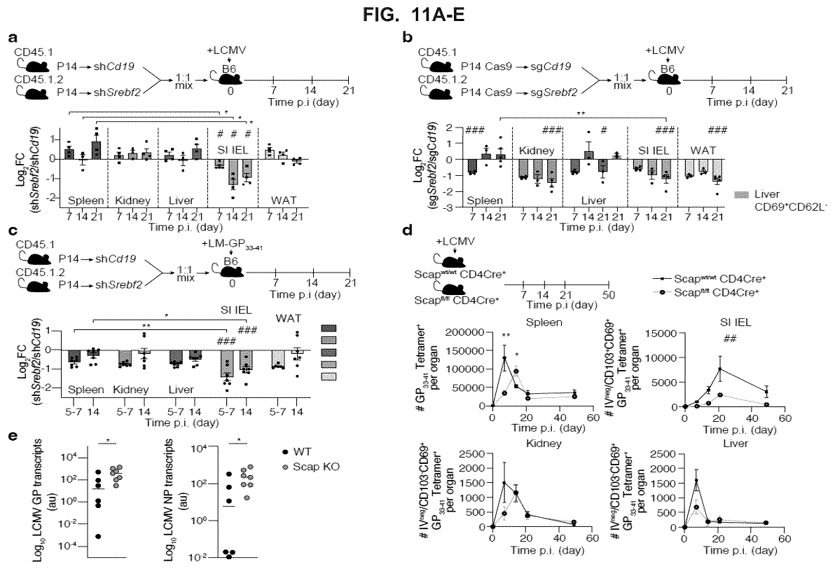

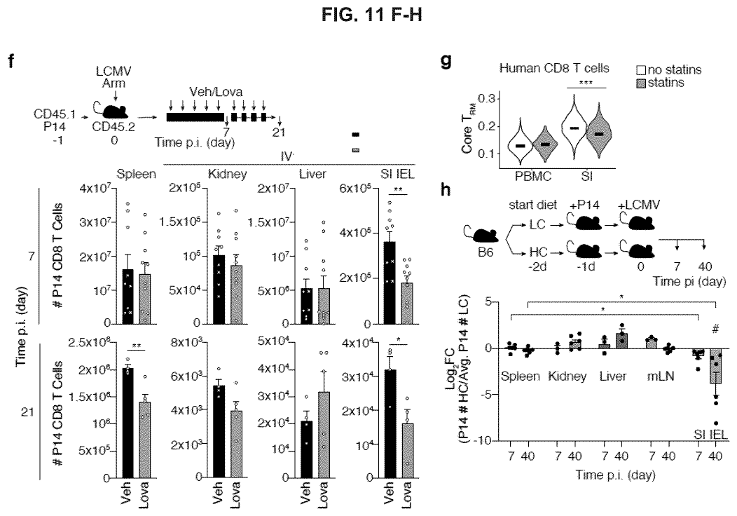

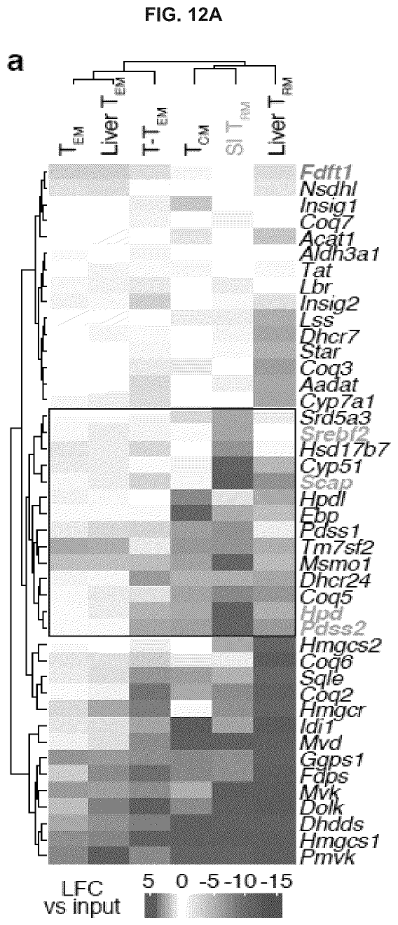

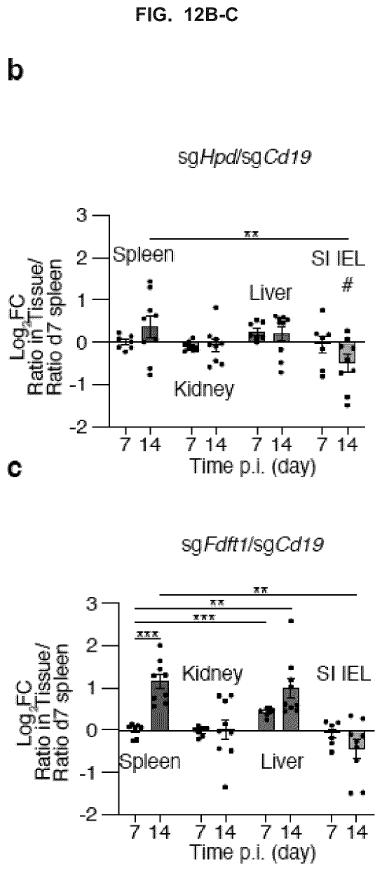

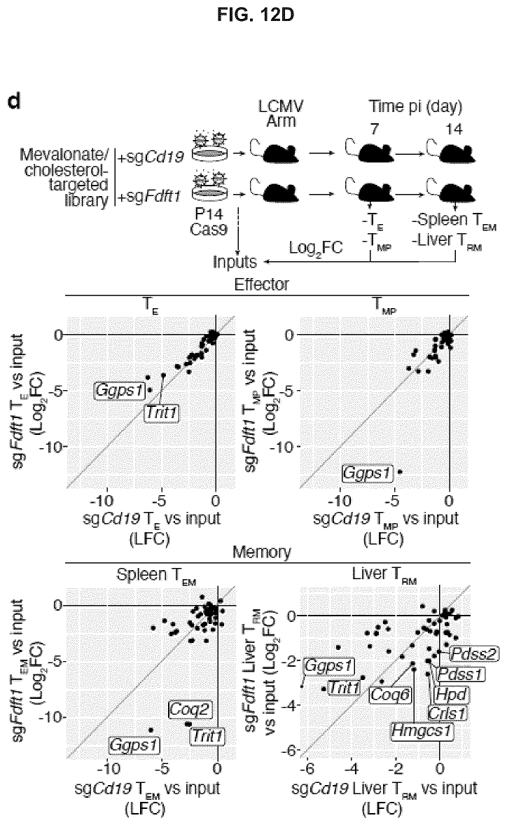

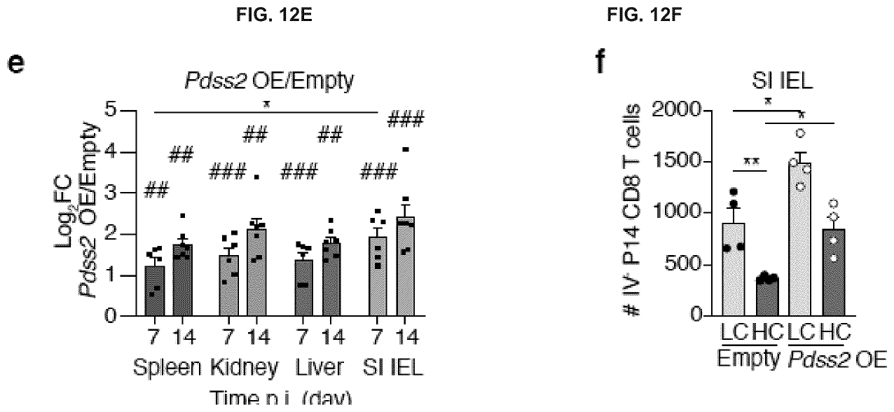

FIG. 11, or FIG. 2 of Example 1. An Srebp2-dependent metabolic program controls SI T_{RM} formation.

a-c, Ratio of transduced transferred P14 CD8 T cells (a and b), or P14 Cas9eGFP CD8 T cells (b), harvested from indicated tissues at indicated times after LCMV (a and b), or LM-GP₃₃₋₄₁ (c) infection, evaluated by flow cytometry. d, Number of total Tet⁺ cells in the spleen, and total Tet⁺ T_{RM} cells in the SI, kidney, and liver of Scap^{fl/fl} Cd4-Cre⁺ and Scap^{wt} Cd4-Cre⁺ at indicated times after LCMV infection. e, LCMV titers measured by qPCR in the SI of WT and Scap KO mice previously infected with LM-GP_{33-41 3} days after LCMV rechallenge. NP, nucleoprotein. GP, glycoprotein. f, Number of total P14 CD8 T cells isolated from indicated tissues at days 7 and 21 after LCMV infection in mice treated with vehicle or Lovastatin (Lova). g, T_{RM} scores of human CD8 T cells of the blood and SI profiled by scRNAseq grouped by statin use; no statins=8 donors, statins=5 donors. h, Ratio of total P14 CD8 T cells from indicated tissues in mice fed high versus low-cholesterol-containing diet. Data are geometrical distributions of single-cell gene expression values with median values (g) or mean+/−s.e.m. and representative of two independent experiments (a, b, d, e and f), or pooled from two independent experiments (c, e, f, and h), with a total of n=7 (a), n=7 (b), n=7 (c), n=8 (d), n=6 in WT and n=7 in Scap KO (e), n=9 (day 7) and n=4 (day 21) (f), and n=3 (mLN, kidney, and liver day 7, and liver day 40), and n=6 (rest) (h) mice. Two-sided unpaired (f), and paired t-Test, (a, b, c, d, f, g, and h), two-sided Mann-Whitney U Test (e). *P<0.05, **P<0.01, ***P<0.005. Two-sided one-sample t-Test (a-c, and h) #P<0.05, ##P<0.01, ###P<0.005. Two-way ANOVA (d) ##P<0.01.

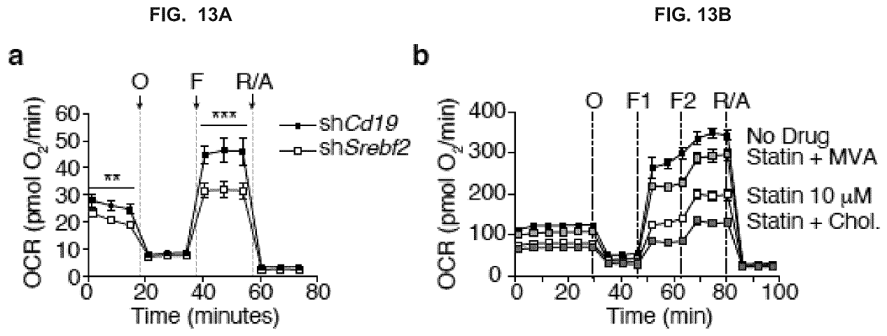

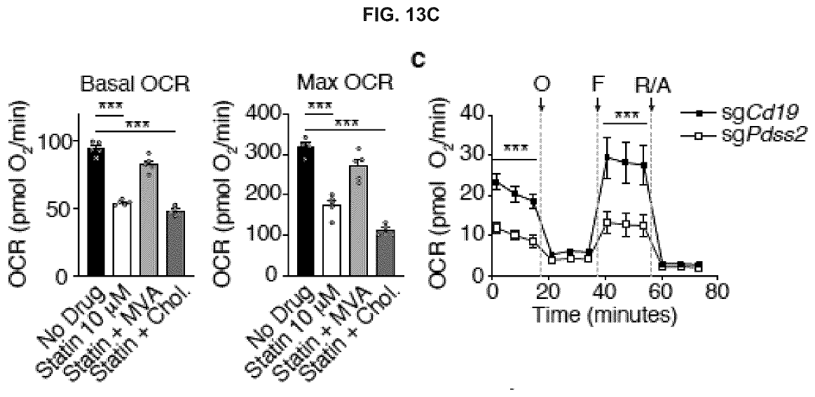

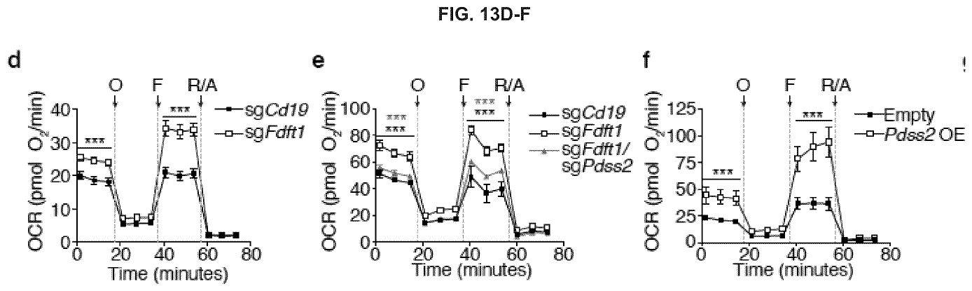

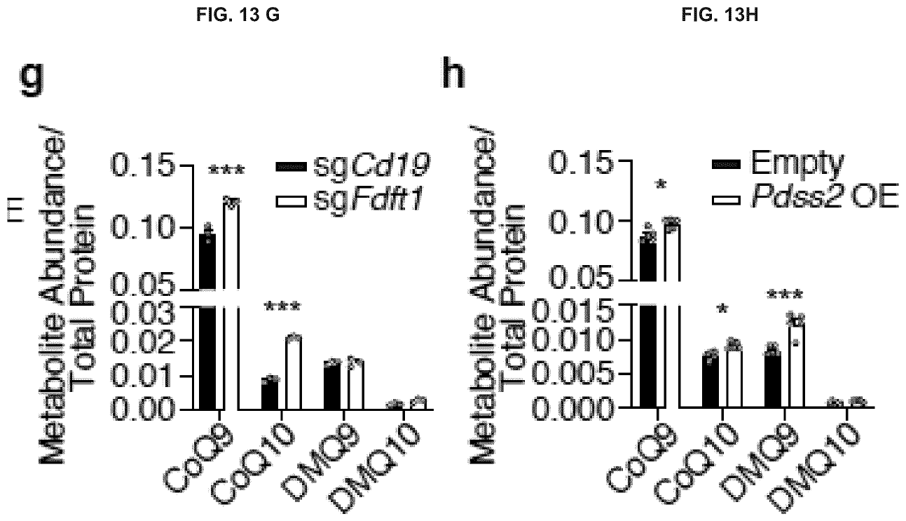

FIG. 13, or FIG. 3 of Example 1. Non-steroidal products of the mevalonate/cholesterol synthesis pathway mediate T_{RM} adaptations.

a, Unsupervised hierarchical clustered heatmap of gene essentiality scores for genes related to the mevalonate/cholesterol synthesis pathway in the cellular populations profiled in our in vivo CRISPR/Cas9-mediated loss-of-function screen. Blue and red denote positive and negative regulators of memory CD8 T cell formation, respectively. b,c, Ratio of indicated transduced transferred P14 Cas9eGFP CD8 T cells, sgHpd (b) and sgFdft1 (c), harvested from indicated tissues at indicated times after LCMV infection, evaluated by flow cytometry. d, Targeted in vivo CRISPR/Cas9-mediated loss-of-function screen of mediators of the effect of Fdft1 deletion on CD8 T cell memory formation. Data visualization represents the enrichment as Log₂ FC of sgRNA frequencies in sgCD19 outputs versus sgCd19 input (x axis), and sgFdft1 outputs versus sgFdft1 input (y axis), for each effector and memory CD8 T cell subset. e, Ratio of transduced transferred P14 CD8 T cells harvested from indicated tissues at indicated times after LCMV infection, evaluated by flow cytometry. f, Total cell numbers of Empty and Pdss2 OE SI IEL P14 CD8 T cells in mice fed low or high cholesterol-containing diets. Data are mean+/−s.e.m. and representative or pooled from at least two independent experiments (b, c, e, and f), or one experiment (d), with a total of n=7 (day 7) and n=9 (day 14) (b, c), n=9 (c), n=6 (d), n=6 (day 7) and n=7 (day 14) (e), n=4 (f) mice. Two-sided unpaired t-Test (b, c, e, and f) *P<0.05, **P<0.01, ***P<0.005. Two-sided one-sample t-Test (b, c, and e) #P<0.05.

FIG. 13, or FIG. 4 of Example 1. Pdss2 expression is required and sufficient to promote mitochondrial respiration and CoQ synthesis in CD8 T cells.

a, Oxygen consumption rate (OCR) of in vitro activated shCd19 and shSrebf2 CD8 T cells subjected to the MITOSTRESS™ test (Seahorse). Oligomycin (O), FCCP (F), Rotenone/Antimycin A (R/A). b, Oxygen consumption rate (OCR) of in vitro activated CD8 T cells treated with statin (simvastatin) in combination with mevalonolactone (MVA) or cholesterol (Chol.) and subjected to the MITOSTRESS™ test (Seahorse). c, OCR of in vitro-activated sgCd19 and sgPdss2 Cas9eGFP CD8 T cells subjected to the MITOSTRESS™ test (Seahorse). d, OCR of in vitro activated sgCd19 and sgFdft1 Cas9eGFP CD8 T cells subjected to the MITOSTRESS™ test (Seahorse). e, OCR of in vitro activated sgCd19, sgFdft1, and sgFdft1 Pdss2 Cas9eGFP CD8 T cells subjected to the MITOSTRESS™ test (Seahorse). f, OCR of in vitro activated empty vector control (Empty) and Pdss2 OE CD8 T cells subjected to the MITOSTRESS™ test (Seahorse). g, Quantification of CoQ and demethoxyubiquinone (DMQ) species in in vitro activated sgCd19 and sgFdft1 Cas9eGFP CD8 T cells normalized to total protein content. h, Quantification of CoQ and DMQ species in in vitro activated Empty and Pdss2 OE CD8 T cells normalized to total protein content. Data are mean+/−s.e.m. and representative of at least two independent experiments (a-h), with a total of n=4 (a), n=5 (b), n=3 (c), n=2 (e), n=3 (f), n=3 (g), n=5 (h) cell replicates. Two-way ANOVA (a, c, d, e, and f). Two-sided unpaired t-Test (b, g, and h) *P<0.05, **P<0.01, ***P<0.005.

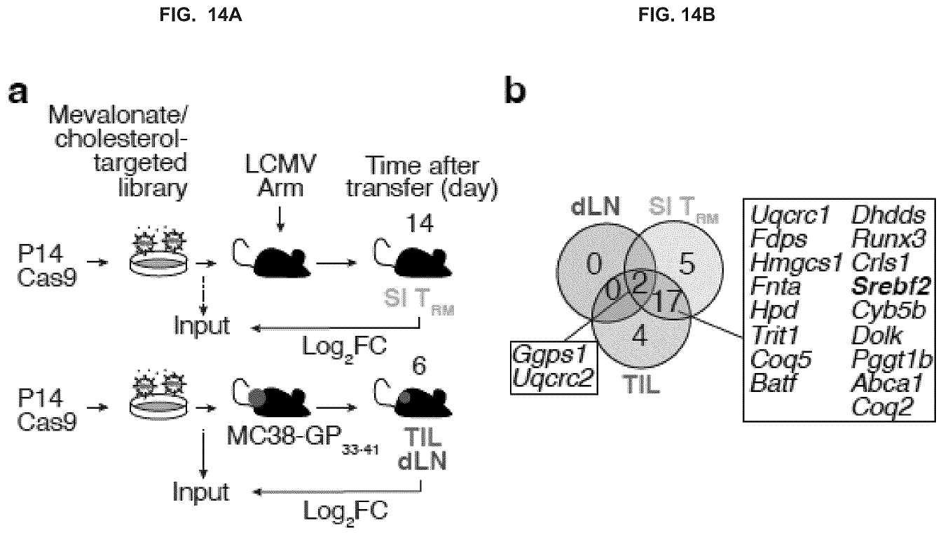

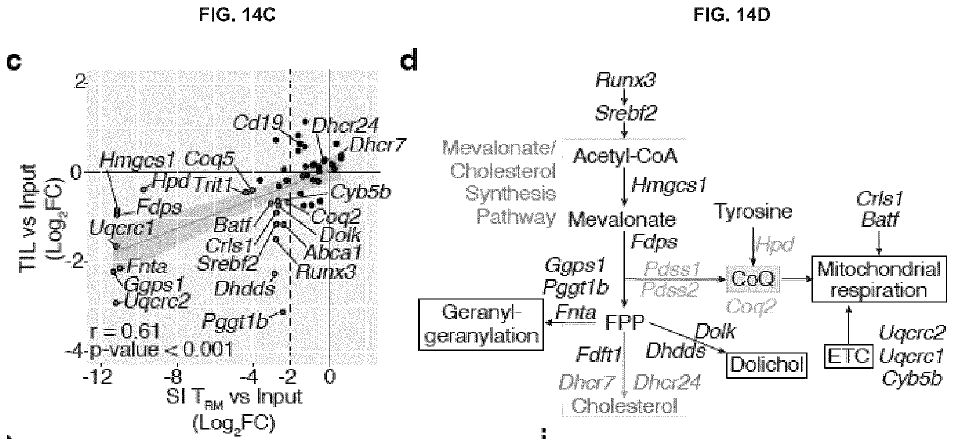

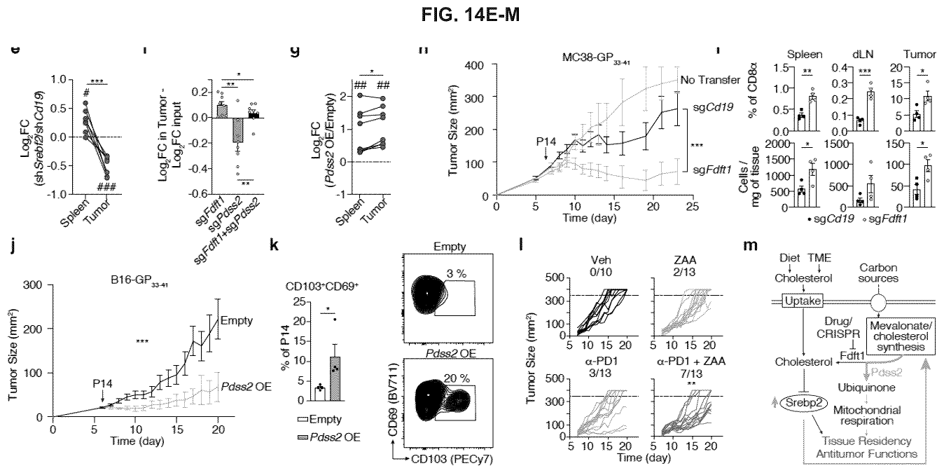

FIG. 14, or FIG. 5 of Example 1. Increased production of non-steroidal products of the mevalonate/cholesterol synthesis pathway is a common requirement for SI T_{RM} and TIL

a, Targeted in vivo screen of mevalonate/cholesterol synthesis pathway genes for SI T_{RM} formation in LCMV infection, and CD8 T cell accumulation in dLN and MC38-GP₃₃₋₄₁ tumors. b, Venn diagram of significant genes (fdr<0.01 for dLN and TIL, fdr<0.01 and Log₂ FC<−2 for SI T_{RM}) from (a). c, Log₂ FC of sgRNA frequencies for each screen from a. d, Metabolic pathway diagram displaying genes required for SI T_{RM} and TIL formation. e, Ratio of transduced transferred P14 CD8 T cells harvested from the spleen and MC-38-GP₃₃₋₄₁ tumors, evaluated by flow cytometry 5-7 days after adoptive cell transfer. f, Ratio of KO P14 Cas9eGFP CD8 T cells vs control (sgCd19) in MC38-GP₃₃₋₄₁ tumors 7 days after adoptive cell transfer. g, Ratio of Pdss2 OE vs Empty P14 CD8 T cells in the spleen and MC38-GP₃₃₋₄₁ tumors 5-7 days after adoptive cell transfer. h, MC38-GP₃₃₋₄₁ tumor growth curves of mice receiving indicated transduced P14 Cas9eGFP CD8 T cells. i, Cell frequencies and total 30 numbers of transduced P14 Cas9eGFP CD8 T cells in MC38-GP₃₃₋₄₁ tumors, dLN, and spleens 4 days after adoptive cell transfer. j, B16-GP₃₃₋₄₁ tumor growth curves in mice adoptively transferred with indicated transduced P14 CD8 T cells. k, Cell frequencies in Empty and Pdss2 OE P14 CD8 T cells in MC38-GP₃₃₋₄₁ tumors. 1, B16-GP₃₃₋₄₁ tumor growth curves of mice receiving vehicle control, ZAA, αPD-1, or ZAA and αPD-1. m, Proposed model of the Srebp2-dependent metabolic programming of SI T_{RM} and TIL. Data are mean+/−s.e.m. and representative or pooled from of at least two independent experiments (e-1), with a total of n=4 (SI T_{RM}) and n=5 (tumor) (a-c), n=9 (e), n=8 (f), n=8 (g), n=3 (no transfer), n=15 (sgCd19), and n=16 (sgFdft1) (h), n=8 (i), n=9 (sgCd19) and n=7 (sgPdss2) (j), n=13 (k), n=10 (veh) and n=13 (rest) (1) mice, and one experiment (a-c), with n=3-5 mice pooled in each sample (a-c). Two-sided unpaired (f and i) and paired (e, g, and k) t-Test. Two-way ANOVA (h and j). Pearson correlation (r) (c). Fisher's exact test (1). *P<0.05, **P<0.01, ***P<0.005. Two-sided one-sample t-Test (e and g) #P<0.05, ###P<0.005.

### EXTENDED DATA FIGURE LEGENDS

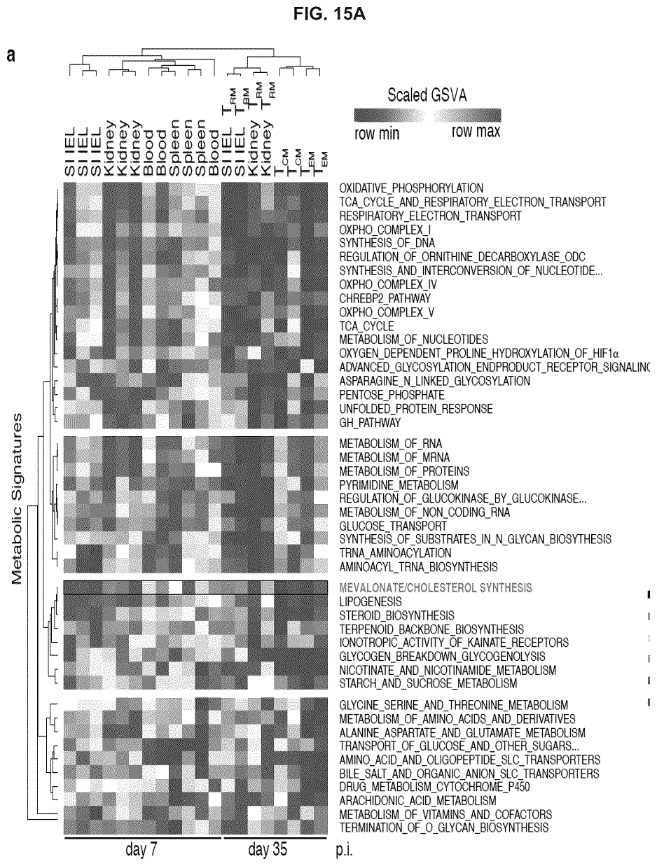

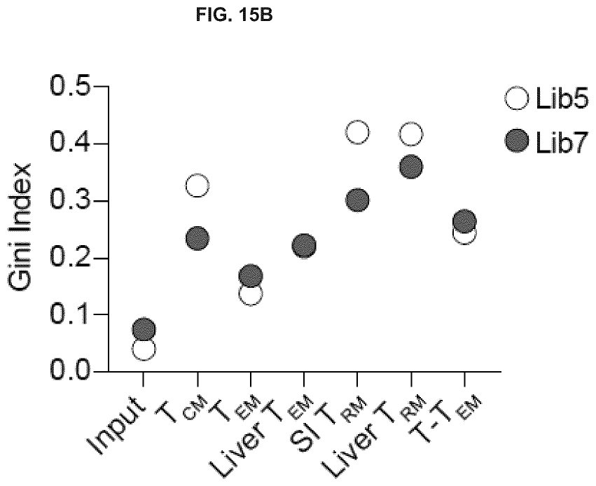

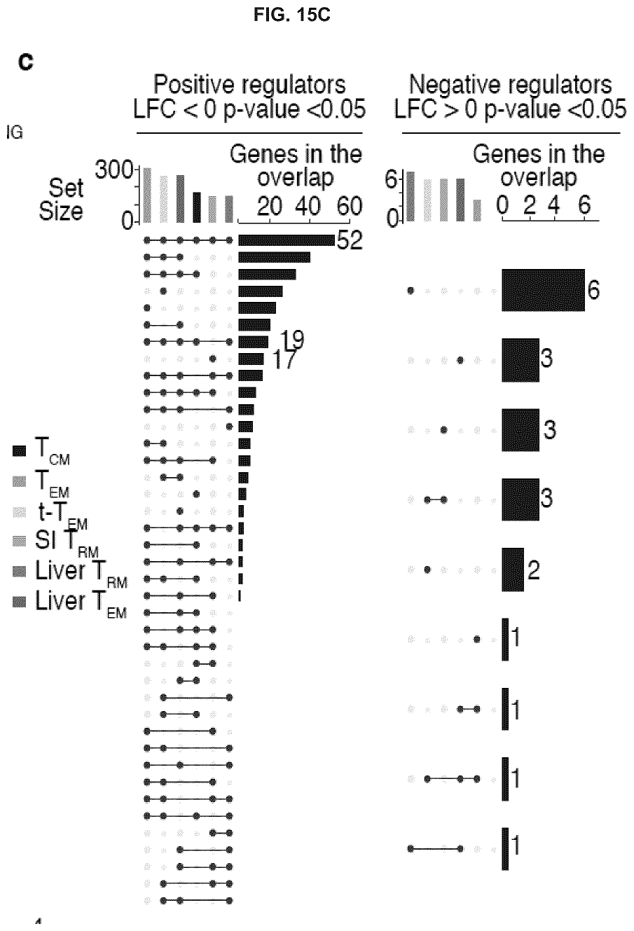

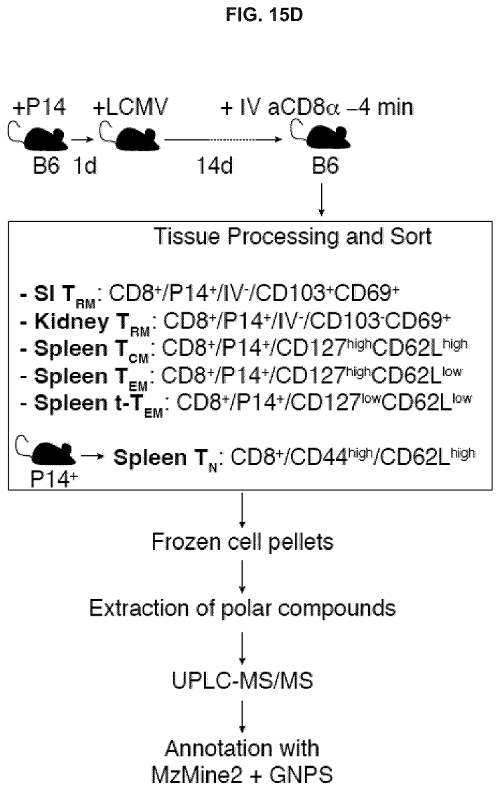

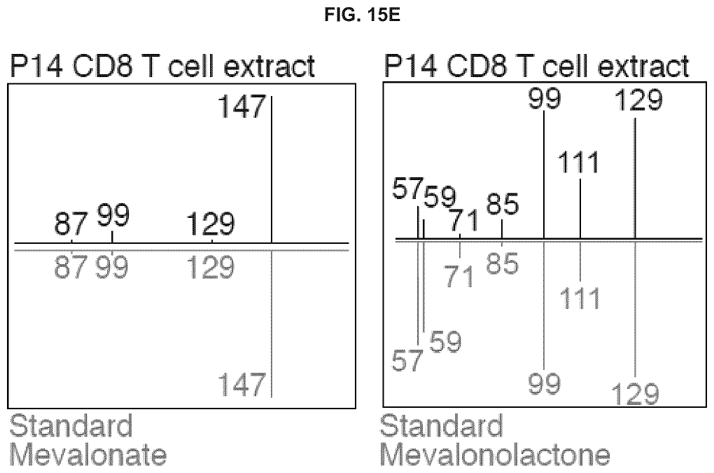

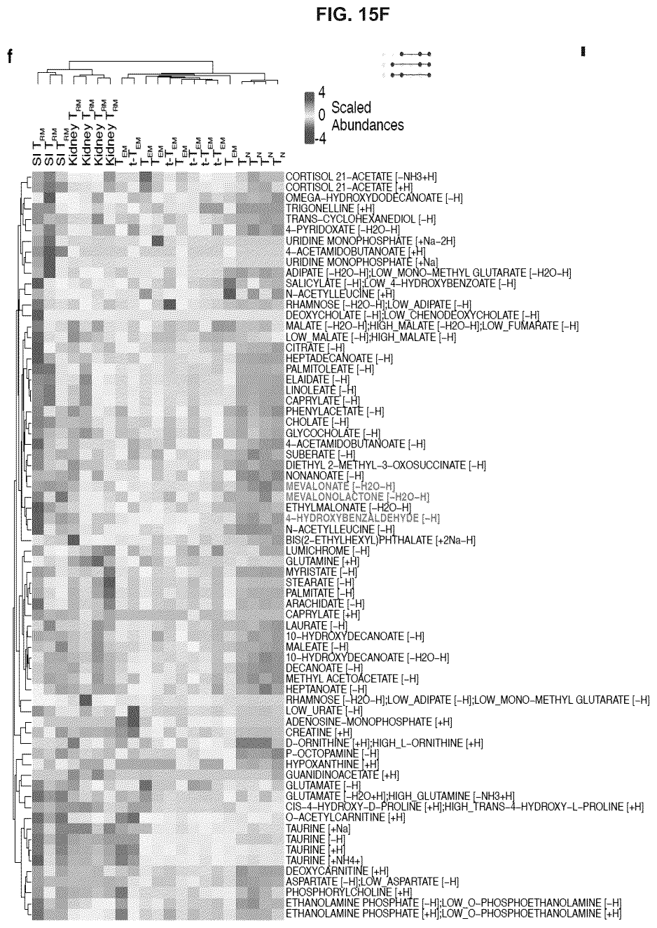

FIG. 15, or Extended Data FIG. 1. Functional genetics in vivo, metabolomics and transcriptional analysis of ex vivo populations of memory CD8 T cells identify a graded upregulation of a Srebp2-dependent metabolic programming across T_{RM}.

a, Unsupervised hierarchical clustering heatmap of scaled Gene set variation analysis (GSVA) scores for metabolic signatures across samples, p.i., post-infection. GSE107278. b, sgRNA library heterogeneity reported as Gini Index values. c, Upset plot showing intersection sizes and set sizes for positive and negative regulators of each P14 CD8 T cell population in the in vivo CRISPR/Cas9-mediated loss-of-function screen. d, Sample acquisition and analysis workflow for LC-MS/MS-based untargeted metabolomics of ex vivo populations of CD8 T cells in the context of LCMV infection. Gating strategy included for each cell type analyzed. e, Tandem mass spectra matching against commercial standards of mevalonate and mevalonolactone. Tandem mass spectra derived from the most abundant ion for each compound. Black spectra are derived from cell lysate, red mirrored spectra from commercial standard. f, Unsupervised hierarchical clustering of scaled relative abundances of identified annotated metabolites of P14 cells profiled by untargeted metabolomics at day 13 post LCMV infection.

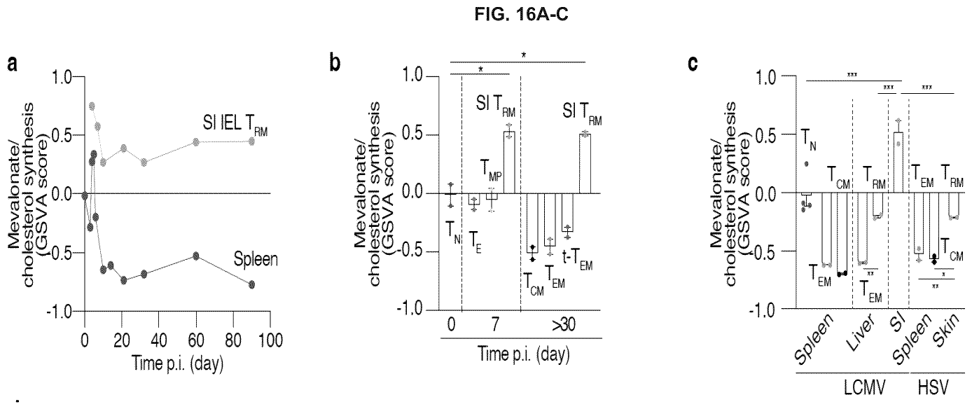

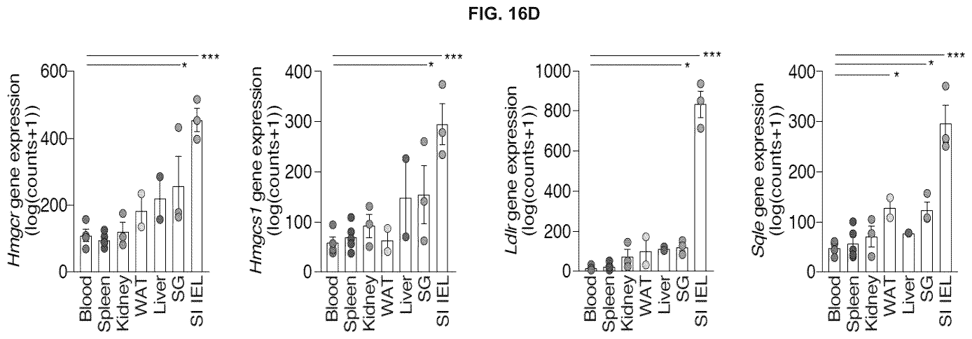

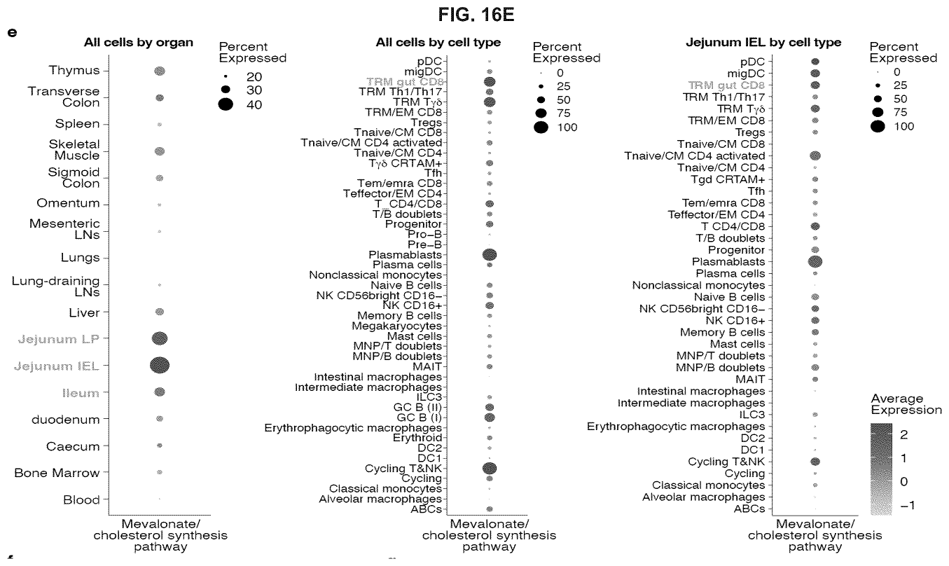

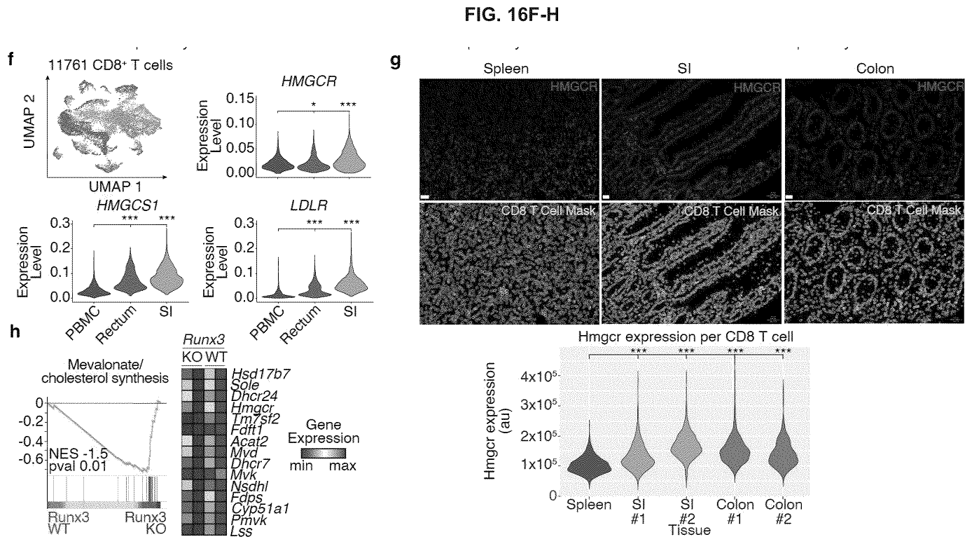

FIG. 16, or Extended Data FIG. 2. Upregulation of the mevalonate/cholesterol synthesis pathway in mouse and human T_{RM}.

a, Mevalonate/cholesterol synthesis GSVA scores from averaged single cell expression of spleen and SI P14 at indicated time points after LCMV infection profiled by scRNASeq, GSE131847. b, Mevalonate/cholesterol synthesis GSVA scores for indicated subsets of CD8 T cells in the spleen or SI in the context of LCMV infection, GSE157072. c, Mevalonate/cholesterol synthesis GSVA scores for indicated subsets of CD8 T cells at the indicated tissues in the context of LCMV and Herpes Simplex Virus (HSV), GSE70813. d, Gene expression of indicated genes in P14 CD8 T cell populations from indicated tissues >30 days after LCMV infection, GSE182276 e, Mevalonate/cholesterol synthesis pathway scores for all human immune cells grouped by indicated tissues (left), cell types (middle), or cell types of the Jejunum SI (right), from the tissue immune cell scRNAseq atlas. f, Expression of selected genes on human CD8 T cells from PBMC, rectum, and SI samples, pooled from 13 healthy donors, GSE125527. g, HMG-CoA reductase (HMGCR) protein expression in CD8 T cells from human spleen, SI, and colon by immunofluorescence staining. Quantification of total HMGCR by CD8 T cell area. Donor ID (#). Scale bar, 20 μm. h, GSEA of the mevalonate/cholesterol synthesis in Runx3 KO or control CD8 T cells GSE106107. Data are pseudo-bulk averaged values (a), mean+/−s.e.m. (b, c, and d), or geometric distribution (f and g), with a total of n=2 (b), n=2-4 (c), n=2-5 (d) mice, and n=1-2 (g) samples. Two-sided unpaired t-Test (b, c, d, f, and g), and Gene Set Enrichment Analysis (h). *P<0.05, **P<0.01, ***P<0.005.

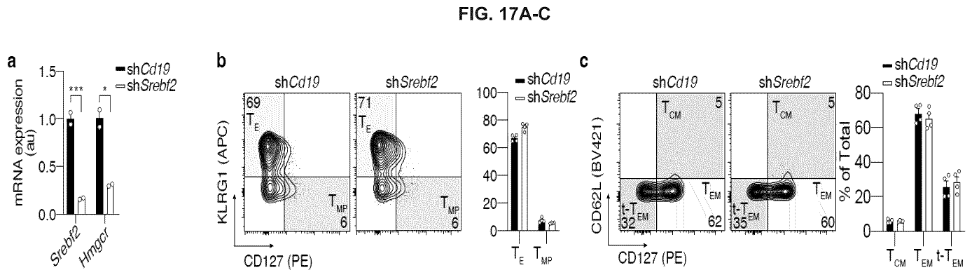

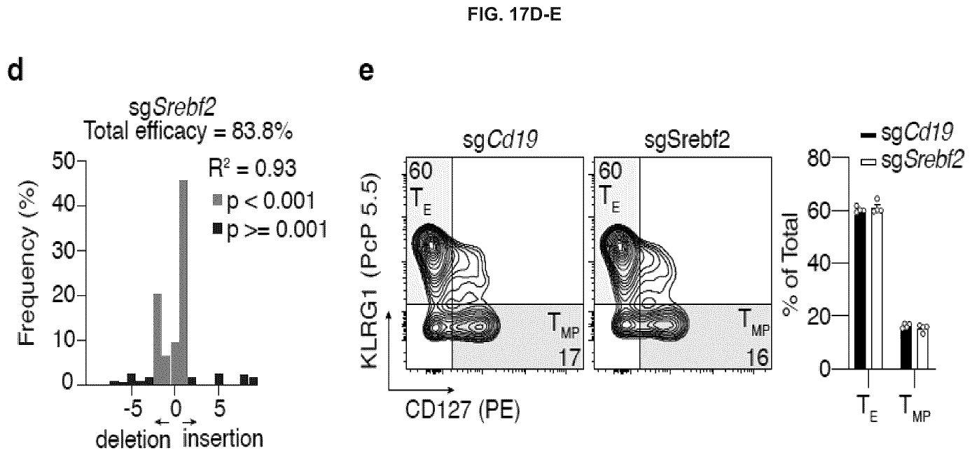

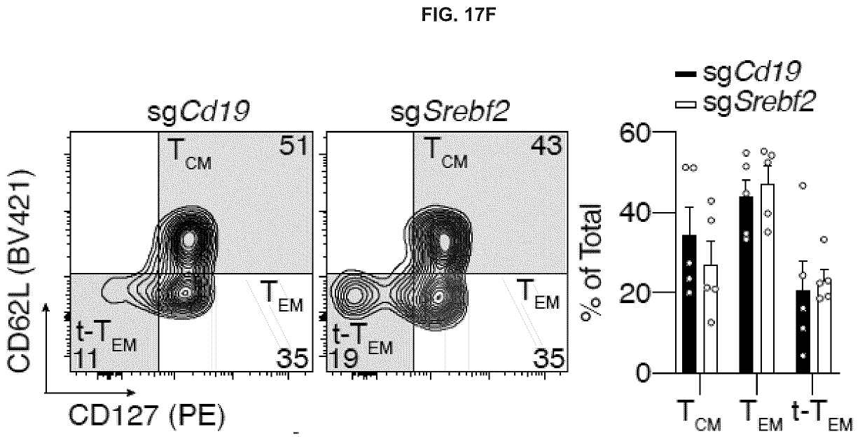

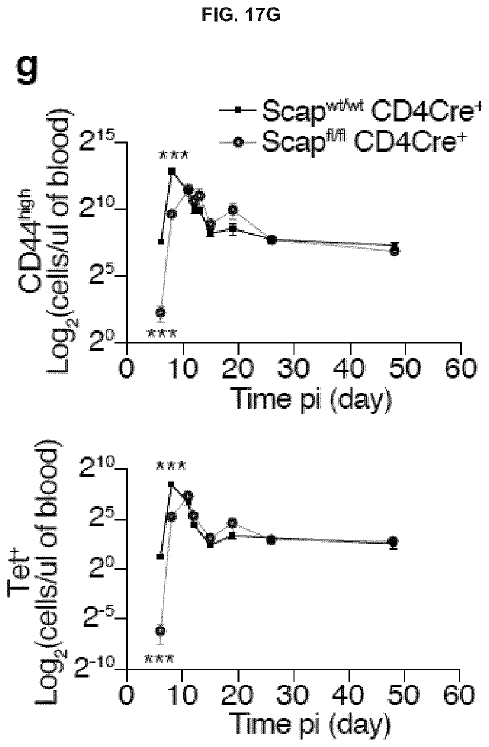

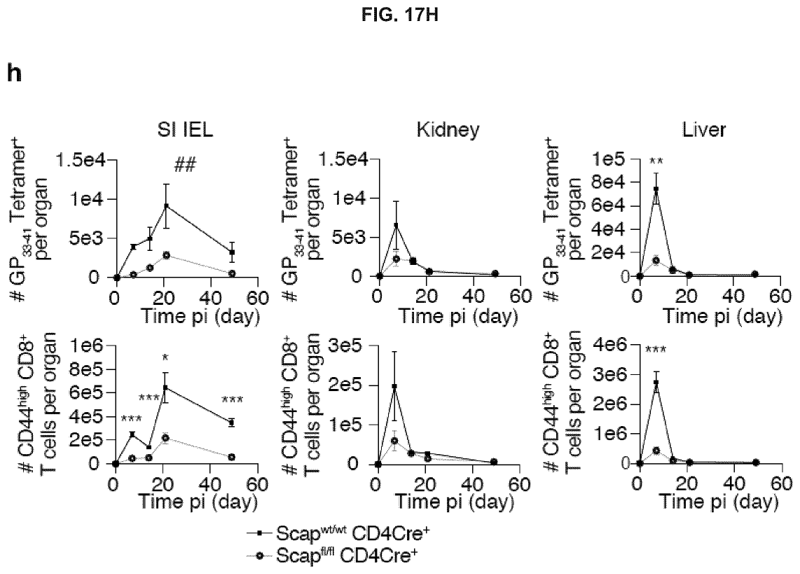

FIG. 17, or Extended Data FIG. 3. A Srebp2-dependent metabolic program controls SI T_{RM} formation.

a, Quantification of gene expression by qPCR of shRNAmir control (shCd19) or shSrebf2 shRNAmir P14 CD8 T cells before adoptive transfer. b, Frequency of effector shRNAmir control (shCd19) or shSrebf2 shRNAmir P14 CD8 T cell populations in the spleen 7 days after LCMV infection. c, Frequency of memory shRNAmir control (shCd19) or shSrebf2 shRNAmir P14 CD8 T cell populations in the spleen 21 days after LCMV infection. d, CRISPR/Cas9-mediated indel efficiency of the Srebf2 sgRNA construct on sorted transduced P14 cells before adoptive transfer. e, Frequency of effector sgCd19 or sgSrebf2 P14 cell populations in the spleen 7 days after LCMV infection. f, Frequency of memory sgCd19 or sgSrebf2 P14 cell populations in the spleen 21 days after LCMV infection. g, Total cell quantification of CD44^{high} CD8 T cells (left) and Tet⁺ CD8 T cells (right) in the blood of Scap WT or Scap KO at different time points after LCMV infection. h, Total cell quantification of Tet⁺ CD8 T cells (upper) and CD44^{high} CD8 T cells (lower) in SI, kidney, and liver of Scap WT or Scap KO at different time points after LCMV infection. i, Ratio of congenically distinct P14 CD8 T cells transduced with a constitutively active nuclear form of human Srebp2⁵⁶ (nSrebp2) or an empty vector (Empty) from indicated tissues 7 days after LCMV infection. j, Flow cytometry analysis of cholesterol content by Filipin stain (left), and LDLR expression in P14 CD8 T cells transduced with a bicistronic construct encoding Thy1.1 alone or Thy1.1 and nSrebp2 (right). P14 CD8 T cells from the kidney, liver, WAT, and SI were gated on the IV⁻ population (h and i). Data are mean+/−s.e.m. and representative of at least two independent experiments, with a total of n=2 (a), n=4 (b-e), n=5 (f), n=6 (WT) and n=7 (Scap KO) (g and h), n=4 (i and j) mice and n=2 (d) cell replicates. Two-sided unpaired (a, d, g, and h) and paired (j) t-Test. *P<0.05, **P<0.01, ***P<0.005. Two-sided one-sample t-Test (i) #P<0.05, ##P<0.01, ###P<0.005. Two-way ANOVA (h) ##P<0.01.

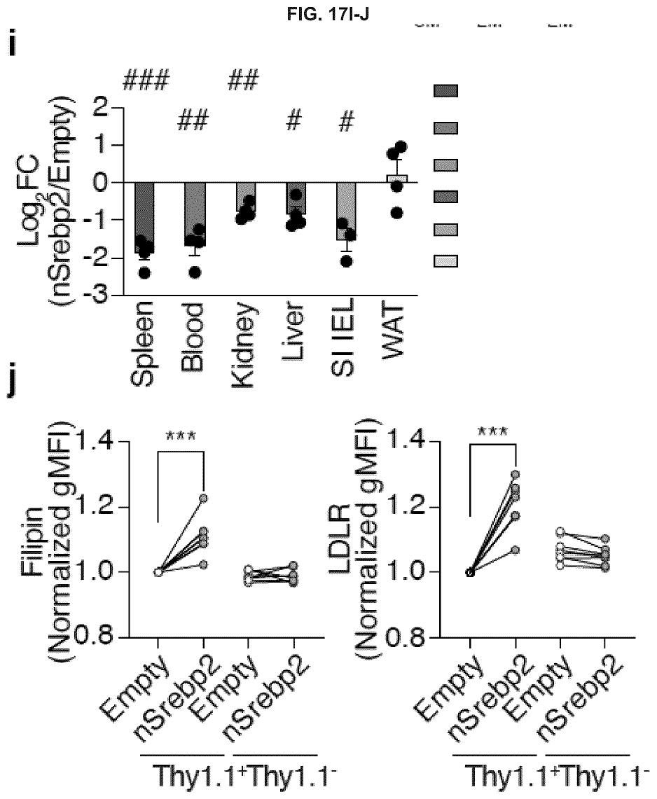

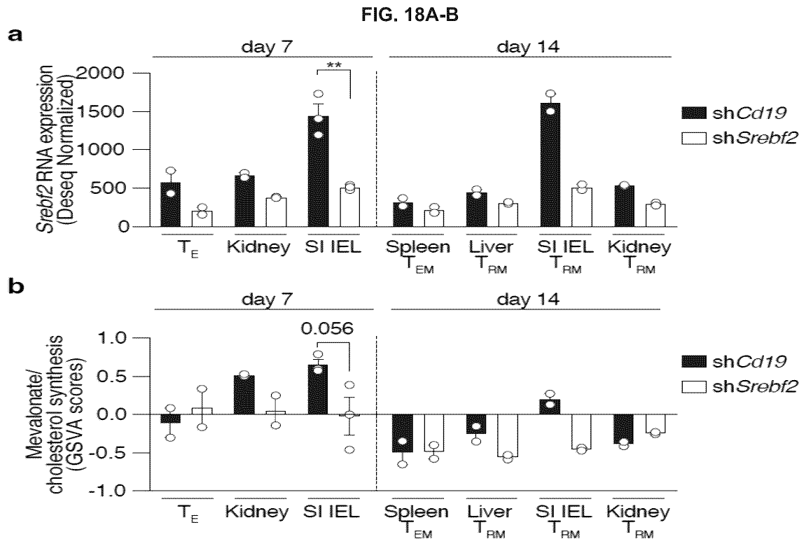

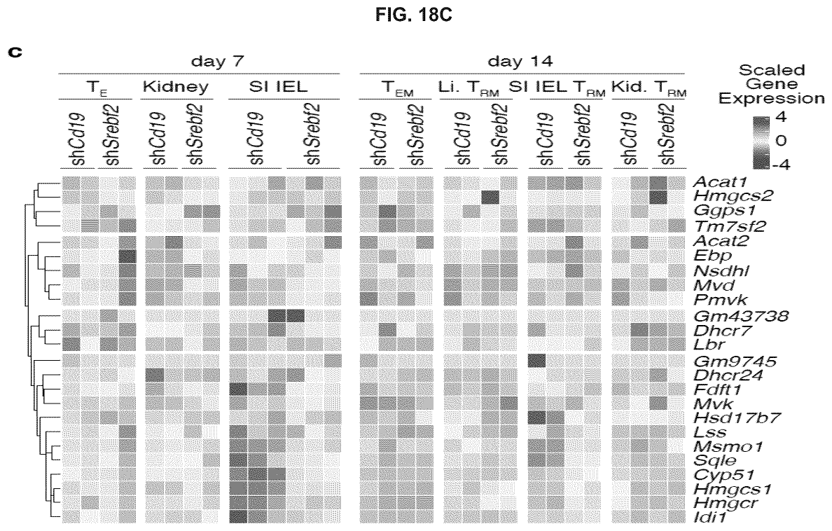

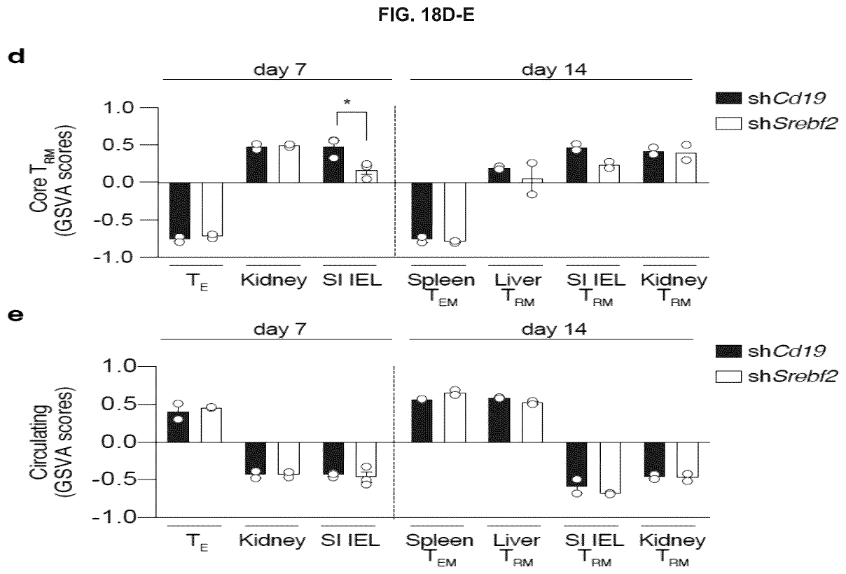

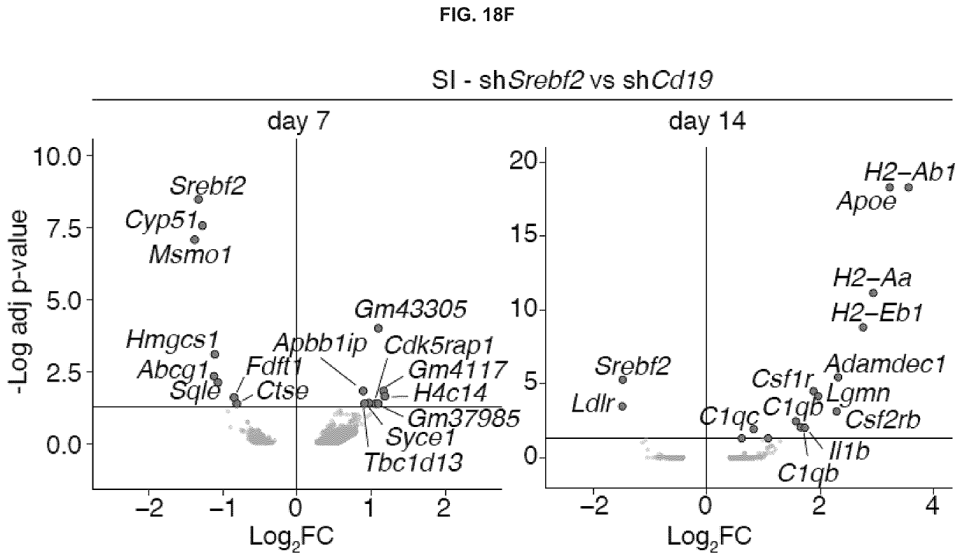

FIG. 18, or Extended FIG. 4. Limited effect of Srebp2 on transcriptional programs of CD8 T cell differentiation.

a, Srebf2 expression values from the bulk ULI-RNAseq analysis of shRNAmir control (shCd19) and Srebf2 shRNAmir P14 CD8 T cells from the spleen, kidney, liver, and SI from day 7 and 14 after LCMV infection. b, Mevalonate/cholesterol synthesis pathway GSVA scores from the bulk ULI-RNAseq analysis of shRNAmir control (shCd19) and Srebf2 shRNAmir P14 CD8 T cells from the spleen, kidney, liver, and SI from day 7 and 14 after LCMV infection. c, Unsupervised hierarchically clustered heatmap of mevalonate/cholesterol synthesis pathway gene expression values from the bulk ULI-RNAseq analysis of shRNAmir control (shCd19) and Srebf2 shRNAmir P14 CD8 T cells from the spleen, kidney, liver, and SI from day 7 and 14 after LCMV infection. d, Core T_{RM} GSVA scores from the bulk ULI-RNAseq analysis of shRNAmir control (shCd19) and Srebf2 shRNAmir P14 CD8 T cells from the spleen, kidney, liver, and SI from day 7 and 14 after LCMV infection. e, Circulating GSVA scores from the bulk ULI-RNAseq analysis of shRNAmir control (shCd19) and Srebf2 shRNAmir P14 CD8 T cells from the spleen, kidney, liver, and SI from day 7 and 14 after LCMV infection. f, Volcano plots of differentially expressed genes between shCd19 and shSrebf2 SI P14 CD8 T cells at days 7 and 14 after LCMV infection, adjusted (adj) p-value. P14 CD8 T cells from the kidney, liver and SI were gated on the IV⁻ population (a-f). T_{E} were defined as KLRG1^{high}CD127^{low} P14 CD8 T cells. Data are mean+/−s.e.m. (a, b, d, and e) from one experiment with n=2-3 (a-f), where every sample is 1×10³ cells pooled from 2-3 mice. Two-sided unpaired t-Test (a, b, d, and e), and Deseq2 DEG testing with Benjamini-Hochberg multiple test correction. *P<0.05, **P<0.01, ***P<0.005.

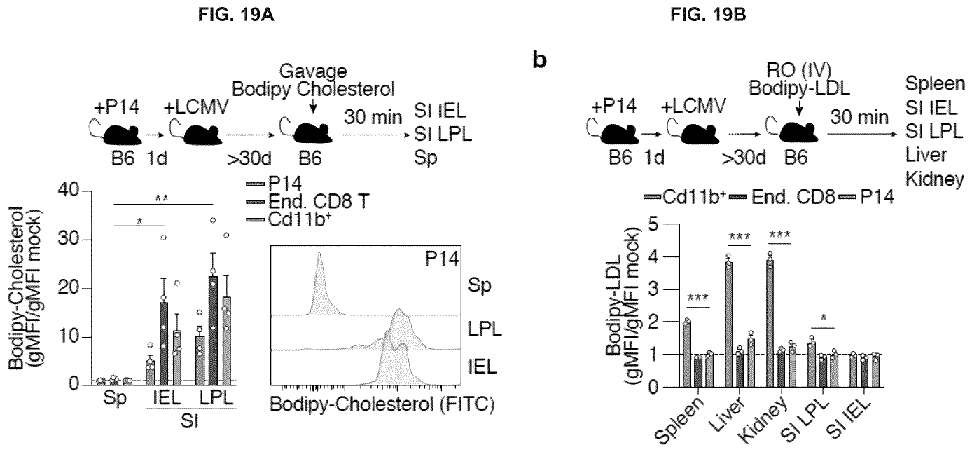

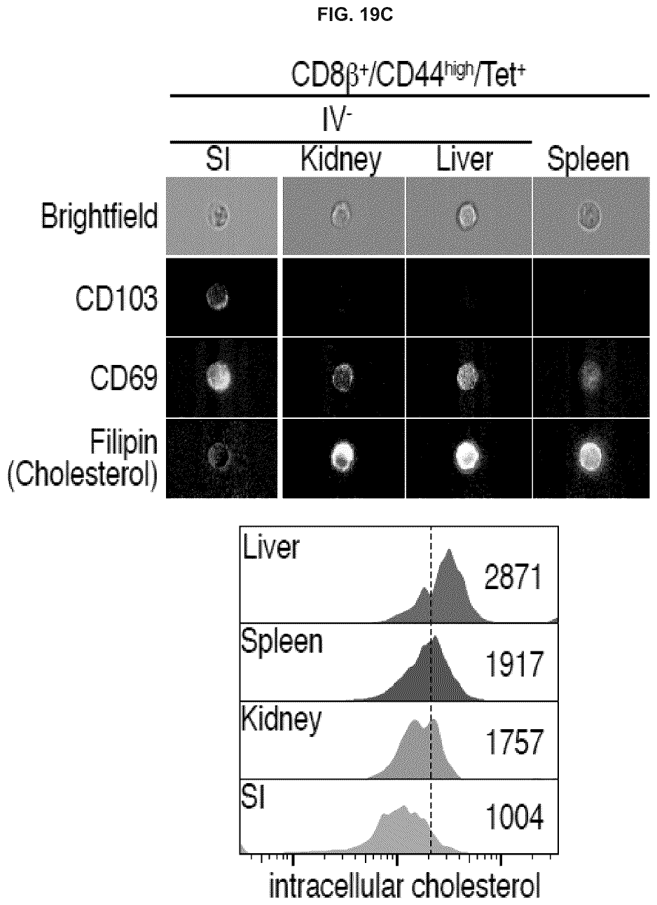

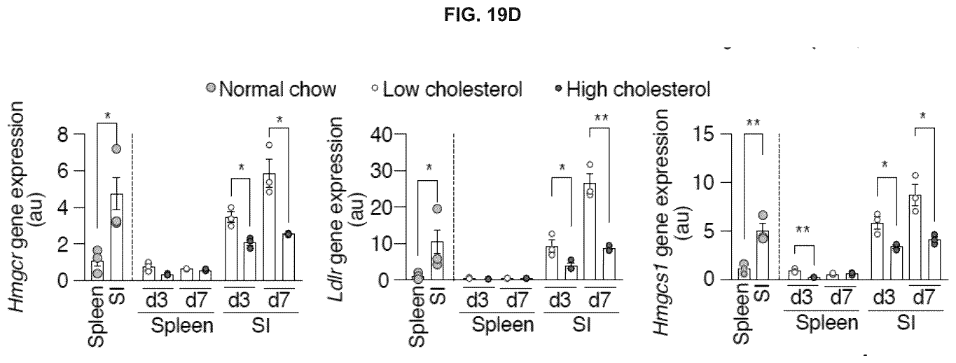

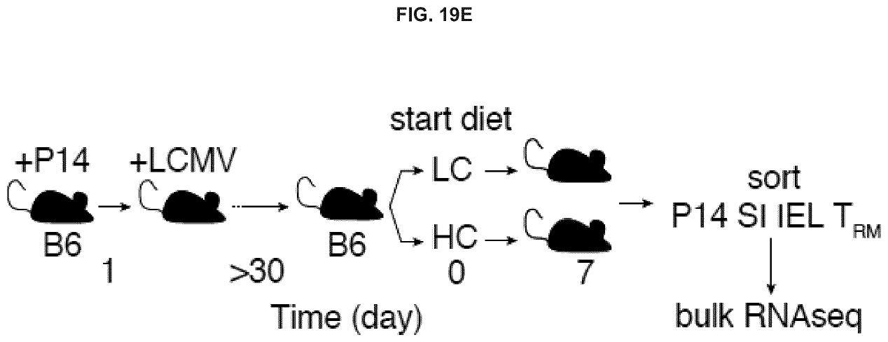

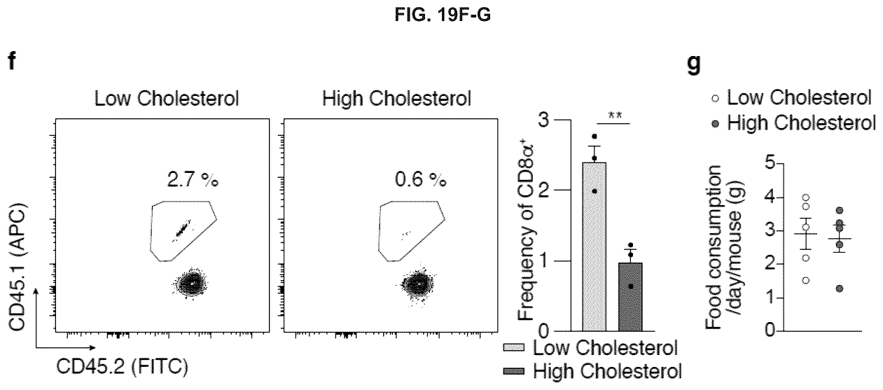

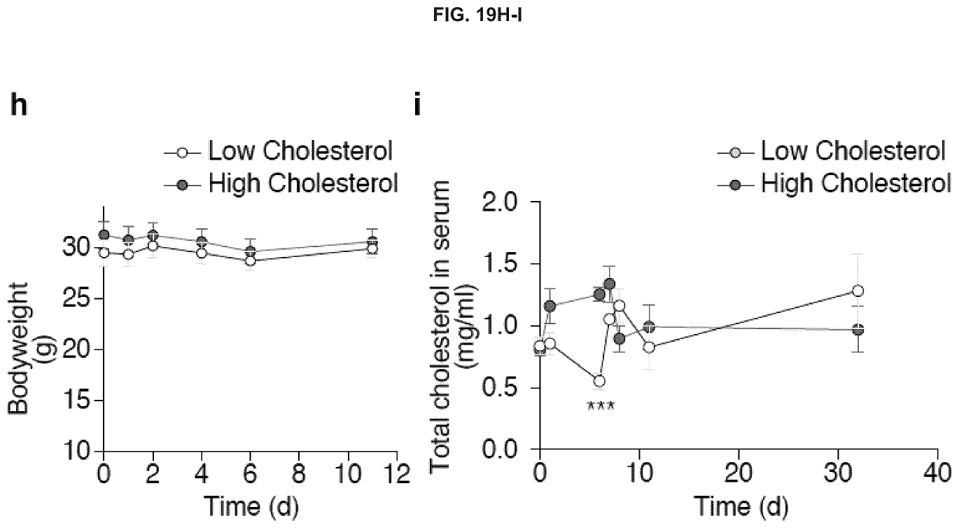

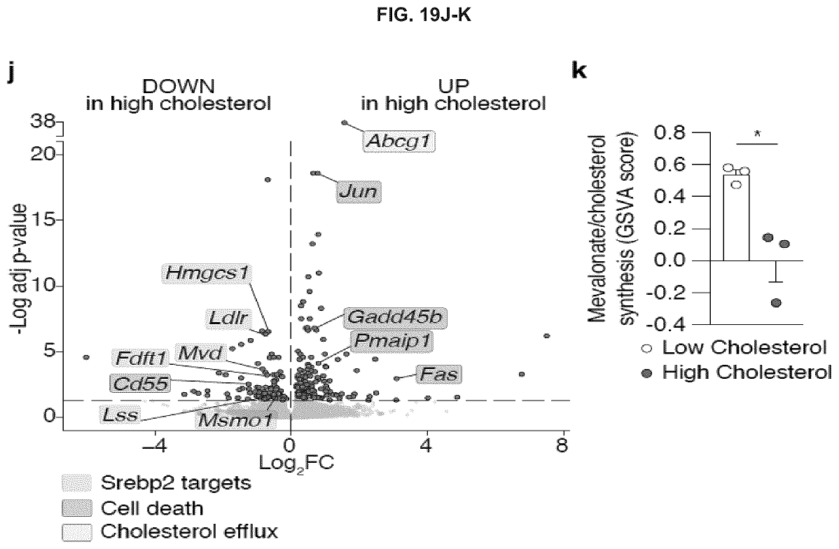

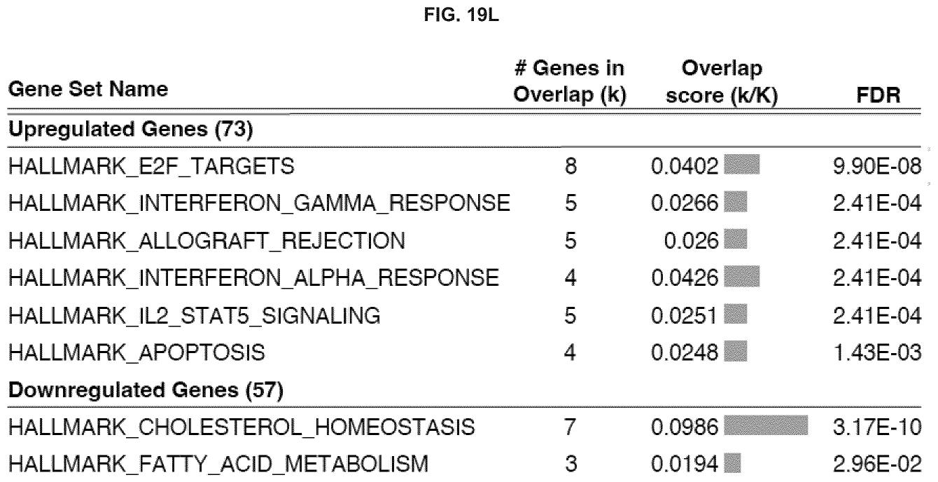

FIG. 19, or Extended Data FIG. 5. SI T_{RM} uptake and sense dietary cholesterol.

a, Quantification by flow cytometry of in vivo incorporation of Bodipy-cholesterol on indicated cell populations after 10 minutes after oral administration of Bodipy-cholesterol to memory P14 mice. b, Quantification by flow cytometry of incorporation of Bodipy-LDL on indicated cell types of the spleen 20 minutes (min) after IV delivery to memory P14 mice. c, Quantification of intracellular cholesterol by filipin staining in Tet⁺ populations of CD8 T cells for various tissues >30 days post LCMV infection measured by imaging flow cytometry. d, Gene expression analysis by qPCR of selected Srebp2 targets on CD8 T cells from the spleen and SI of mice on regular chow or subjected to low and high cholesterol-containing diets. e, Experimental design to measure transcriptional changes induced by dietary cholesterol on established SI T_{RM} by ULI-RNAseq, f, SI T_{RM} frequencies from total CD8 T cells after 7 days of dietary intervention. g, Food consumption per day per mouse subjected to a low or high-cholesterol diet. h, Bodyweight of mice subjected to low or high cholesterol-containing diets. i, Total cholesterol in serum in mice subjected to low or high cholesterol-containing diets. j, Volcano plot of differentially expressed genes in SI T_{RM} from mice fed a high vs a low cholesterol-containing diet. k, Mevalonate/cholesterol synthesis GSVA scores in SI T_{RM} from mice fed a high versus low cholesterol-containing diet. 1, Gene pathway analysis of top upregulated and top downregulated genes between SI T_{RM} from mice fed a high versus low cholesterol-containing diet (Molecular Signatures Database (MSigDB), Hallmark gene sets). P14 CD8 T cells and CD8β⁺/CD44^{high}/Tet⁺ from the kidney, liver and SI were gated on the IV⁻ population (a, b, c, e, f, j, k, and 1). Data are mean+/−s.e.m. and representative of at least two independent experiments, with a total of n=4 (a), n=3 (b, d), n=5 (c), n=3 (f) mice, or pooled from at least two independent experiments with a 30 total of n=5-10 (g-i) mice, or performed once (j-1) with a total of n=3 samples each sample is 1×10³ SI T_{RM} pooled from 2~3 mice (j, k). Two-sided unpaired t-Test (a, b, d, f, i, and k), and Deseq2 DEG testing with Benjamini-Hochberg multiple test correction (j). *P<0.05, **P<0.01, ***P<0.005.

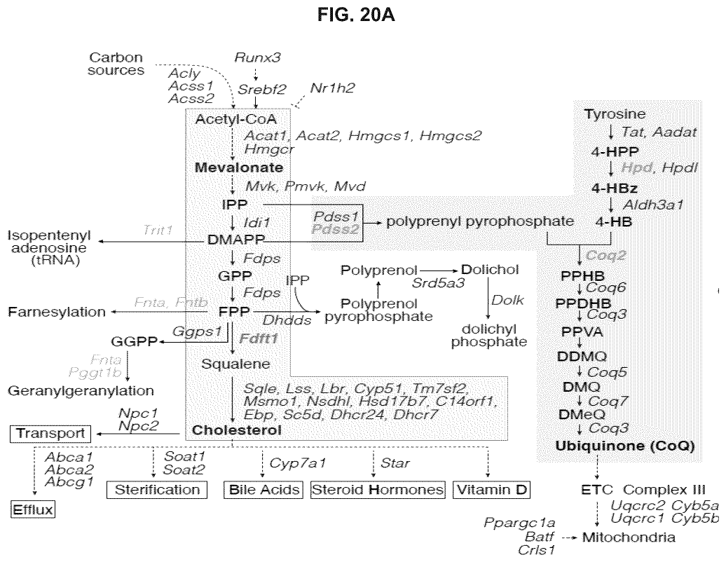

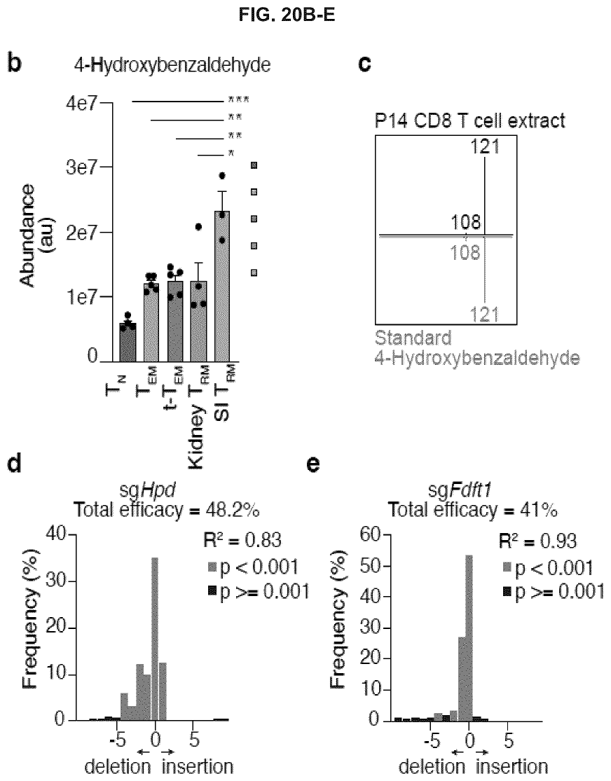

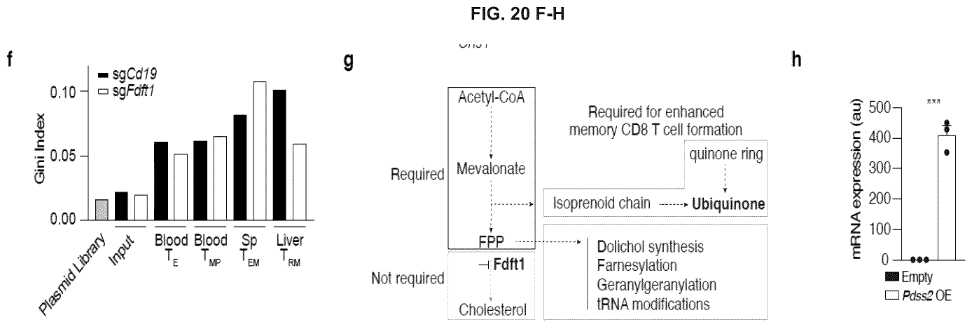

FIG. 20, or Extended Data FIG. 6. Mevalonate/cholesterol synthesis pathway intermediates sustain the production of non-steroidal metabolites.

a, The mevalonate/cholesterol synthesis pathway with adjacent and downstream metabolic routes. Enzymes not included in the first CRISPR/Cas9-mediated loss-of-function screen in grey, downstream products and/or fates of cholesterol are boxed, key measured metabolites in bold, non-steroidal metabolites in blue, key enzymes are bold and colored by in vivo effect of deletion in P14 CD8 T cells, gain-of-function in red, loss-of-function in blue, dashed lines indicate indirect or unknown mode of regulation. b, Abundance of 4-hydroxybenzaldehyde profiled by LC-MS/MS of ex vivo populations of P14 cells 13 days after LCMV infection. c, Tandem mass spectra matching against commercial standards of 4-hydroxybenzaldehyde. Tandem mass spectra derived from the most abundant ion for each compound: m/z=121.0295 for 4-hydroxybenzaldehyde [M-H], black spectra are derived from cell lysate, red mirrored spectra from commercial standard. d, CRISPR/Cas9-mediated indel efficiency of the Hpd sgRNA construct on sorted transduced P14 cells before adoptive transfer. e, CRISPR/Cas9-mediated indel efficiency of the Fdft1 sgRNA construct on sorted transduced P14 CD8 T cells before adoptive transfer. f, Gini index scores of sgRNA representation in each library. g, Schematic illustration of the contribution of mevalonate/cholesterol pathway intermediates to non-steroidal products. h, Quantification of Pdss2 mRNA by qPCR in transduced Empty and Pdss2 OE P14 CD8 T cells. Data are mean+/−s.e.m. (b and h), and representative of at least two independent experiments (d, e, and h), with a total of n=2 (d, e), and n=3 (h) cell replicates, or one experiment (b), with n=3 (SI), n=4 (TN), and n=5 (rest) (b) samples, where each sample contains cells pooled from 2 to 5 mice. Two-sided unpaired t-Test (b, d, e and h). *P<0.05, **P<0.01, ***P<0.005.

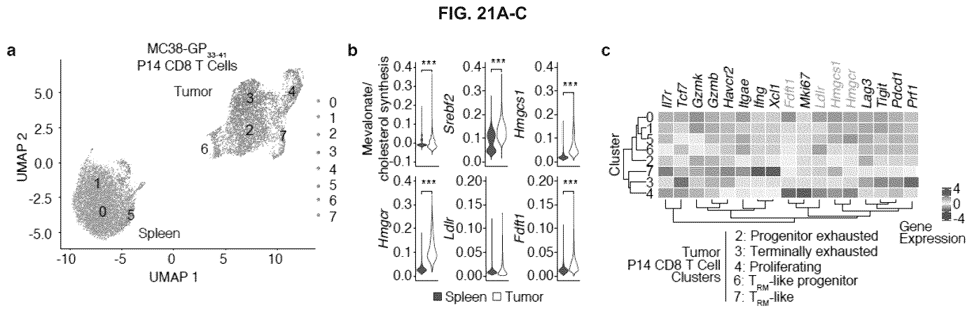

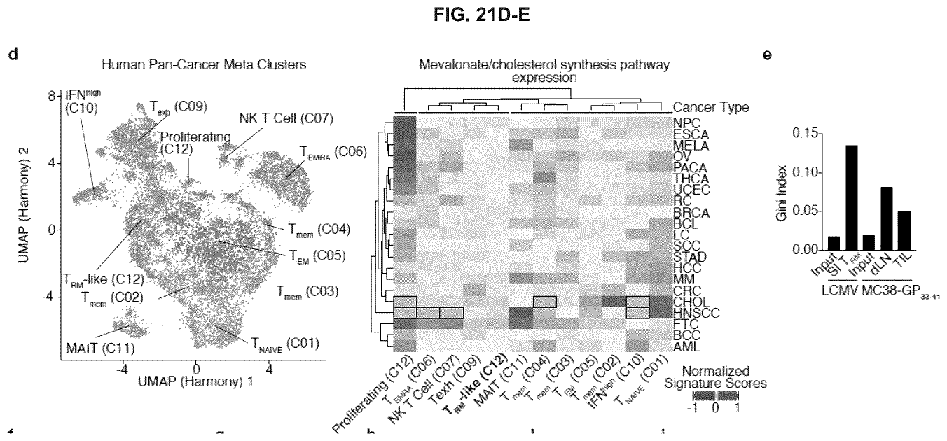

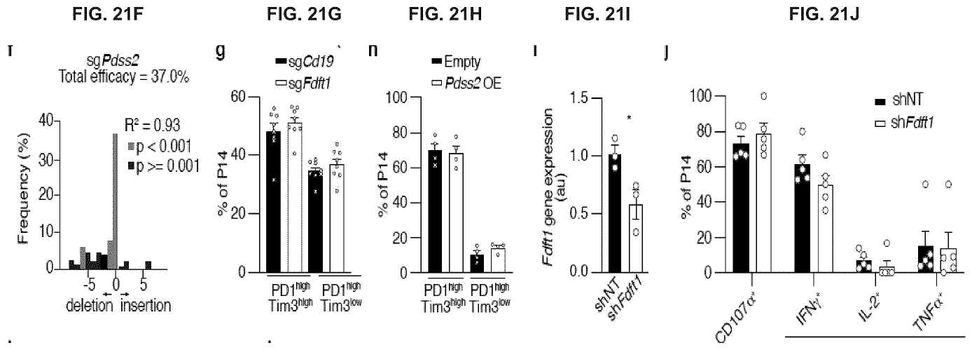

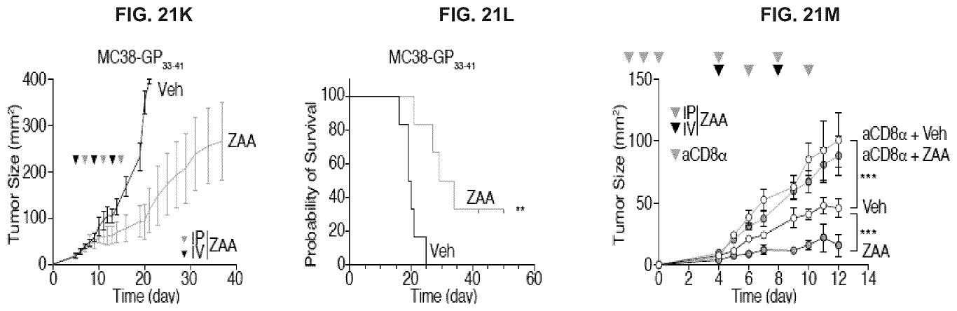

FIG. 21, or Extended Data FIG. 7. Increased production of non-steroidal products of the mevalonate/cholesterol synthesis pathway is a common requirement between SI T_{RM} and TIL

a, Dimensionality reduction plot by UMAP of P14 CD8 T cells profiled from spleens and subcutaneously implanted MC38-GP₃₃₋₄₁ tumors by scRNAseq 7 days after adoptive cell transfer. b, Mevalonate/cholesterol synthesis signature values and expression of indicated genes in P14 CD8 T cells from the spleen and MC38-GP₃₃₋₄₁ tumors profiled by scRNAseq. MAGIC-imputed gene values are shown. c, Hierarchically clustered heatmap of scaled averaged gene expression values by cluster of P14 cells profiled from spleens and MC38-GP₃₃₋₄₁ tumors by scRNAseq 7 days after adoptive transfer. d, Dimensionality reduction plot by UMAP of CD8 T cell metaclusters (left) with “meta.cluster.coarse” labels and hierarchically clustered heatmap of mevalonate/cholesterol synthesis pathway scores grouped by “meta.cluster.coarse” labels and cell type. NPC, nasopharingeal carcinoma, ESCA, esophageal cancer, LC, lung cancer, HCC, hepatocellular carcinoma, CHOL, cholangiocarcinoma, RC, renal carcinoma, CRC, colorectal cancer, AML, acute myeloid leukemia, BCL, B-cell lymphoma, MM, multiple myeloma, HNSCC, head and neck squamous cell carcinoma, THCA, thyroid carcinoma, MELA, melanoma, BCC, basal cell carcinoma, SCC, squamous cell carcinoma, BRCA, breast cancer, STAD, stomach adenocarcinoma, PACA, pancreatic cancer, OV, ovarian cancer, FTC, fallopian tube carcinoma, UCEC, uterine corpus endometrial carcinoma. EMRA, effector memory CD45RA⁺, mem, memory. e, sgRNA library heterogeneity reported as Gini Index values. f, CRISPR/Cas9-mediated indel efficiency of the Pdss2 sgRNA construct on sorted transduced P14 CD8 T cells. g, Frequency of PD1^{high}Tim3^{high} and PD1^{high}Tim3^{low} subpopulations for sgCd19 and sgFdft1 Cas9 P14 CD8 T cells in MC38-GP₃₃₋₄₁ tumors at day 5-7 after adoptive transfer. h, Frequency of PD1^{high}Tim3^{high} and PD1^{high}Tim3^{low} subpopulations for Empty and Pdss2 OE P14 CD8 T cells in MC38-GP₃₃₋₄₁ tumors at day 5-7 after adoptive transfer. i, Quantification of gene expression by qPCR of control (shNT) and Fdft1 deficient (shFdft1) P14 CD8 T cells before adoptive transfer. j, Restimulation capacity of control (shNT) and Fdft1 deficient (shFdft1) P14 TIL 4 days after in vivo transfer treated with GP₃₃₋₄₁ peptide ex vivo. k, MC38-GP₃₃₋₄₁ tumor growth curves of mice receiving ZAA or vehicle. 1, Survival of mice implanted with MC38-GP₃₃₋₄₁ tumors receiving ZAA or vehicle. m, MC38-GP₃₃₋₄₁ tumor growth curves of mice receiving ZAA in combination with CD8 T cell depletion. Data are mean+/−s.e.m. (g, h, i, j, k, and m), raw values (d), geometrical distributions of single-cell gene expression values (b) or averaged gene expression (c and e) and pooled or representative of at least two independent experiments, with a total of n=2 (f) cell replicates, and n=8 (g), n=9 (h), n=3 (i), n=10 (j), n=12/group (k, 1), n=5/group (m) mice. Two-sided unpaired t-Test (b, f and i). Log-rank test (1). Two-way ANOVA (m). *P<0.05, **P<0.01, ***P<0.005.

FIG. 22, or Extended Data FIG. 8. A requirement for the mevalonate/cholesterol synthesis enzymes for TIL persistence and heightened antitumor activity in a melanoma model.

a, GSEA of the mevalonate/cholesterol synthesis pathway in genes pre-ranked by their impact on TIL accumulation in B16-OVA tumors from a published CRISPR/Cas9-mediated loss-of-function screen. b, Dimensionality reduction plot by UMAP of WT and Regnase-1 KO OT-I cells profiled from MC38-GP₃₃₋₄₁ tumors by scRNAseq 7 days after adoptive transfer (left) and unsupervised hierarchical clustering heatmap of selected genes related to CD8 T cell cytotoxicity (Ifng) stemness (Tcf7), proliferation (Mki67), and the mevalonate/cholesterol synthesis pathway (Srebf2, Hmgcs1, Fdft1, Hmgcr, and Ldlr) grouped by cell type and cluster. c, Comparison of the effect of targeting the 50 genes of the mevalonate/cholesterol pathway included in our targeted screen in WT TIL or Regnase-1 KO TIL (sgZc3h12a) from a published CRISPR/Cas9-mediated loss-of-function screen in B16 melanoma tumors³². Relevant enzymes are labeled.

### Methods

### Mice

Mice were maintained in specific-pathogen-free conditions at a temperature between 18° C. and 23° C. with 40-60% humidity and a 12 h-light and 12 h-dark light cycle in accordance with the Institutional Animal Care and Use Committees (IACUC) of the University of California, San Diego (UCSD) and the University of California, Los Angeles (UCLA). All mice were of C57BL6/J background and bred at UCSD, UCLA or purchased from the Jackson Laboratory. P14, Cas9eGFP (stock #026179, The Jackson Laboratory), Thy1.1, and CD45.1 congenic mice were bred in house. Male and female mice were used for experiments, and were age and sex-matched, between 1.5 and 6 months old, and randomly assigned to experimental groups, except for therapeutic and dietary interventions where mice were randomly distributed into different experimental arms. No statistical methods were used to pre-determine sample sizes but our sample sizes are similar to those reported in previous publications from our laboratory and others. No blinding was performed during mouse experiments. Investigators were not blinded to group allocation during data collection and/or analysis. Low (D12102C) and high (D12104C) cholesterol diets were procured from Research Diets, Inc (New Brunswick, NJ). Mice were fed ad libitum for the specified amount of time. Intravascular staining of CD8 T cells was done by administration of 3 μg of anti CD8α, APC-eFluor780 (eBioscience 47-0081-82) in 200 μl of PBS by retroorbital (RO) route. CD8α⁻ (IV⁻) cells were considered to be localized within non-lymphoid tissues². Isolation of IV⁻ P14 CD8 T cells from the liver may exclude resident T cells in the vasculature. All animal studies were approved by the Institutional Animal Care and Use Committees of the University of California, San Diego (UCSD) and performed in accordance with UC guidelines.

### Cell Culture

PlatE cells were acquired from Cell Biolabs (RV-101). MC38-GP₃₃₋₄₁ cell line was generated from the commercially available C57BL/6 murine colon adenocarcinoma MC38 parental cell line (cat. #ENH204-FP, Kerafast (Shirley, MA)). HEK293T cells were a gift from the Chi lab and originally sourced from the ATCC (CRL-3216). The B16-GP₃₃₋₄₁ cell line was a gift from Dr. Alain Lamarre and is derived from C57BL/6 murine B16-F10 skin melanoma. PlatE, MC38-GP₃₃₋₄₁, and B16-GP₃₃₋₄₁ cells were cultured in DMEM+D-glucose supplemented with 10% bovine growth serum, 100 U/mL Penicillin, 100 μg/mL Streptomycin, 292 μg/mL L-glutamine, and 10 mM HEPES. Enriched CD8 T cells were maintained in RPMI+L-glutamine supplemented with 10% fetal bovine serum, 100 U/mL Penicillin, 100 μg/mL Streptomycin, 292 μg/mL L-glutamine, 10 mM HEPES, 1 mM sodium pyruvate (Gibco), 1×MEM NEAA (Gibco), and 55 μM 2-Mercaptoethanol.

### Naive T Cell Transfers and Infection Models

5×10⁴ naive P14 CD8 T cells were transferred intravenously into congenically distinct sex-matched recipient mice, or female P14 cells were transferred into male mice. Recipient mice were subsequently infected intraperitoneally with 2×10⁵ plaque-forming units (PFU) of the Armstrong strain of LCMV or 5000 colony-forming units (CFU) of L. monocytogenes expressing GP₃₃₋₄₁ one day after cell transfer.

### Single Transfer and Co-Transfer Experiments of In Vitro-Activated and Transduced P14 CD8 T Cells

For transfections using retroviral constructs, PlatE cells were seeded in 10-cm dishes at a density of 2.5×10⁵ cells/plate 1 day before transfection in complete DMEM media (10% FBS, 2 mM L-Glutamine, 1× Penicillin/Streptomycin). Transfections were performed with 25 pg plasmid DNA from each pLMPd-Amt, or LsgA clone with TransIT-LT1 (Mirus). Retroviral supernatant was harvested 48 hours (h) and 72 h after transfection. For transductions, negatively enriched naive CD8 T cells from spleen and lymph nodes were activated in 6-well plates coated with 100 μg/ml goat anti-hamster IgG (H+L; Thermo Fisher Scientific), 1 μg/ml anti-CD3 (145-2C11; eBioscience), and 1 μg/ml anti-CD28 (37.51; eBioscience). T cell culture media was removed 18 h after activation and replaced with retroviral supernatant supplemented with 50 μM P-mercaptoethanol (Gibco) and 8 μg/ml polybrene (Millipore) followed by a 1 h spinfection centrifugation at 2,000 rpm and 37° C. One day after transduction, congenically distinct ametrine⁺ T cells were mixed 1:1 and 5×10⁵ total P14 cells were transferred into recipient mice subsequently infected with LCMV. Transduced CD8 T cell 1:1 ratios were validated by flow cytometry prior to adoptive cell transfer into mice.

### Preparation of Single-Cell Suspensions for Flow Cytometry

Isolation of CD8 T cells was performed similarly as described³. SI IEL were prepared through the removal of Peyer's patches and the luminal contents from the entire SI. The SI was then cut longitudinally and into 1 cm pieces, then incubated at 37° C. for 30 minutes in HBSS with 2.1 mg/mL sodium bicarbonate, 2.4 mg/mL HEPES, 8% bovine growth serum, and 0.154 mg/mL of dithioetheritol (EMD Millipore). The kidneys, salivary glands, fat, liver, and tumors were minced into small pieces and then incubated in RPMI with 1.2 mg/mL HEPES, 292 p/mL L-glutamine, 1 mM MgCl2, 1 mM CaCl2), 5% fetal bovine serum, and 100 U/mL collagenase (Worthington) at 37° C. for 30 min. After enzymatic incubations (kidneys, WAT, and livers), tissues were further dissociated over a 70-μm nylon cell strainer (Falcon). Lymphocytes from the small intestine, kidney, salivary gland, and liver were separated on a 44%/⁶⁷% Percoll density gradient. Spleens and lymph nodes were processed with the frosted ends of microscope slides. Red blood cells were lysed with ACK buffer (140 mM NH₄Cl and 17 mM Tris-base, pH 7.4). Blood samples were treated with ACK lysis buffer.

### Antibodies, Intracellular Staining, Flow Cytometry and Cell Sorting

The following antibodies were obtained from TONBO: CD8a (53-6.7, FITC Catalog #35-0081-U500, 1:200 dilution), Invitrogen: CD8a (53-6.7, PerCP-Cy5.5 Catalog #1941169, PE Catalog #2062469, PE-Cy7 Catalog #25-0081-82, 1:200 dilution), PD1 (J43, APC Catalog #47-9985-82, 1:200 dilution), CD127 (A7R34 PE Catalog #12-1271-82, 1:50 dilution), Tim3 (RMT3, PE Catalog #12-5870-82, 1:200 dilution), CD45.2 (104, PE-Cy7 Catalog #25-0454-82, 1:200 dilution), KLRG1 (2F1 PerCP eF710 Catalog #2011186, 1:400 dilution), and TNFα (MP6-XT22, APC Catalog #17-7321-82, 1:100 dilution), eBioscience: CD38 (145-2C11 PE Catalog #12-0031-83, 1:100 dilution), CD80 (H35-17.2 FITC Catalog #11-0083-82, 1:200 dilution), CD11b (M1/70, PE Catalog #12-0112-82, 1:400 dilution), CD45.1 (A20-1.7, APC Catalog #17-0453-82, 1:200 dilution), Thy1.1 (HIS51, FITC Catalog #11-0900-85, 1:1000 dilution), CD44 (IM7, APC Catalog #17-0441-82, 1:400 dilution), and IL-2 (JES6-5H4, PE Catalog #12-7021-82, dilution 1:50), BioLegend: CD8a (53-6.7, PB Catalog #100725, 1:200 dilution), CD62L (MEL-14, BV421 Catalog #104435, BV510 Catalog #104441, 1:400 dilution), CD103 (2E7, PE-Cy7 Catalog #121426, 1:200 dilution), B220 (RA3-6B2, BV711 Catalog #103255, 1:200 dilution), CD44 (IM7, BV711 Catalog #103057, BV510 Catalog #103043, 1:400 dilution), CD69 (H1.2F3, BV711 Catalog #104537, 1:100 dilution), CD45.1 (A20-1.7, BV786 Catalog #110743, BV510 Catalog #110741, 1:200 dilution), Thy1.1 (OX-7 BV421 Catalog #202529, AF647 Catalog #202508, 1:1000 dilution), and IFNγ (XMG1.2, Pacific Blue Catalog 505818, 1:200 dilution), R&D Systems: LDLR (263123, FITC Catalog #263123, PE Catalog #FAB2255P, 1:100 dilution), or BD Biosciences: CD107a (1D4B, FITC Catalog #561069, 1:100 dilution). The H-2Db GP₃₃₋₄₁ tetramer was obtained from the NIH Tetramer Core conjugated to APC or BV421 fluorochromes and used at 1:200 dilution. Dead cell staining was performed with Fixable Viability Dye eFluor780 or eFluor506 (eBiosciences) and used at 1:2000 dilution. For flow cytometry analysis, all events were acquired on a BD LSRFORTESSA X-20™ or a BD LSRFORTESSA™ running FACSDIVA v9.0™ software. Cell sorting was performed on a BD FACSAria™ and flow cytometry analysis was performed on FLOWJO v10™.

### Imaging Flow

Imaging flow cytometry data were acquired with a two-camera AMNIS IMAGESTREAMX MKII™ with 60× objective at Low speed using INSPIRE v6.2.187.0 acquisition software. 15,000 single, in-focus cells were collected per data file. FITC, PE, and PerCP were excited with a 488 nm laser (200 mW) and recorded in camera one. Filipin and BV510 were excited by a 405 nm laser (120 mW) and recorded in camera two. APC and APC-Cy7 were excited by 642 nm laser (150 mW) and recorded in camera two. SSC was recorded in channel 06 (camera one) and the 785 nm laser was set to 1.25 mW. Compensation was calculated with the IDEAS compensation wizard and then manually fine-tuned. Internal filipin intensities were calculated as a new feature on the Filipin channel using a mask based on M01 using Eroded 7 pixels with the IDEAS software. Feature intensities were exported as FCS files and further analyzed on FLOWJO™

LCMV Rechallenge and Quantification of LCMV Titers by qPCR

LCMV rechallenge and quantification of LCMV titers by qPCR to measure protection was done as previously published^{4,5}. In short, mice were infected IV with 5000 CFU of L. monocytogenes expressing GP₃₃₋₄₁. 23 days after the primary challenge, mice were infected intraperitoneally with 2×10⁵ PFU of the Armstrong strain of LCMV. 3 days later, spleens and duodenums from the SI were harvested and flash-frozen in dry ice. RNA extraction from mice tissues was done following homogenization with mortar and pestle in liquid nitrogen, followed by homogenization with the QIASHREDDER™ (Qiagen), followed by RNA purification with the RNAEASY™ kit (Qiagen). cDNA was generated by retrotranscribing 1 μg of total RNA with random hexamer primers (HIGH-CAPACITY CDNA REVERSE TRANSCRIPTION KIT™, Thermo Scientific). Quantification of LCMV genomes was performed by qPCR with 2×SYBR green (Agilent) and the following primers: m18S F (5′-3′):

(SEQ ID NO: 3)
GTAACCCGTTGAACCCCAT,
m18S R (5′-3′):
(SEQ ID NO: 4)
CCATCCAATCGGTAGTAGCG,
LCMV-GP F (5′-3′):
(SEQ ID NO: 5)
CATTCACCTGGACTTTGTCAGACTC,
LCMV-GP R (5′-3′):
(SEQ ID NO: 6)
GCAACTGCTGTGTTCCCGAAA,
and
LCMV-NP F (5′-3′):
(SEQ ID NO: 7)
CAGAAATGTTGATGCTGGACTGC,
LCMV-NP R (5′-3′):
(SEQ ID NO: 8)
CAGACCTTGGCTTGCTTTACACAG.

The 2\hat{ }-DeltaDeltaCt method (see for example, Livak et al, Methods. 2001; 25:402-8), using m18S as housekeeping and WT samples as the reference group, was used to quantify the abundance of LCMV transcripts.

### Mitochondrial Respiration Analysis of CD8 T Cells

Mouse CD8 T cells from the spleen were treated with vehicle or 10 μM simvastatin (Sigma-Aldrich) at the time of activation in combination with 20 μM of Methyl-beta-cyclodextrin (MBCD)-Cholesterol (Sigma) or 10 μM mevalonolactone (Sigma) for 24 hours in complete T cell media. Analysis of in vitro activated and transduced CD8 T cells was performed 24 to 48 hours after transduction following FACS sorting of transduced cells. The SEAHORSE XF HS™ and XF96™ analyzers (Agilent) were used to measure the mitochondrial respiration capacity of CD8 T cells. Cells were seeded at a density of 20,000 cells per well in SEAHORSE XFP CELL CULTURE MINIPLATES™ with inserts without coating (Agilent Technologies) or 300,000 cells/well in CELLTAK™-coated XF96 cell culture microplates (Agilent Technologies). Before seeding, cells were washed once with XF RPMI (Agilent) complete media. Complete media contains freshly added 2 mM glutamine (Gibco), 1 mM Sodium Pyruvate (Gibco), and 10 mM glucose. For the statin experiment, DMEM (Sigma #D5030) supplemented with 8 mM glucose, 2 mM glutamine, 2 mM pyruvate, and 5 mM HEPES was used. Cells were plated in 160 μl of volume, and plates were centrifuged at 300 relative centrifugal force (RCF) for 4 minutes, followed by incubation at 37° C. without CO₂ for 30 minutes to allow for cell attachment and equilibration. Next, the chemicals for the mitochondrial stress test were prepared in assay media and loaded into the cartridge. For the mitochondrial stress test, the following compounds were sequentially injected into each well: 1 M oligomycin, 2 M carbonyl cyanide-4-(trifluoromethoxy)phenylhydrazone (FCCP), or two rounds of 1 μM FCCP injections, 0.5 M rotenone, and 0.5 M antimycin A (all from Agilent Technologies) and measurements were performed at 5-minute intervals. During the experiment, cells were maintained at 37° C. without CO₂. Three to six baseline measurements and three measurements following each injection were taken. Data were analyzed using the SEAHORSE XF WAVE™ software (Agilent Technologies). Statistical analysis was performed using GRAPHPAD PRISM 9.0™ version (GRAPHPAD™ Software). Data were analyzed using a two-tailed paired t-test or a two-way Analysis of Variance (ANOVA) comparing Oxygen consumption rate (OCR) values by sample type and time.

### Histology and Immunofluorescence Staining of Fresh Frozen Mouse Tissues and FFPE Human Tissue

Mouse organs were harvested and rapidly snap-frozen in OCT in plastic molds for cryosection (Tissue-Tek Cryomold). 10 μm slices were obtained using a cryostat and mounted on glass slides, dried for 20 minutes at −20° C., submerged in cold PBS for 5 minutes and fixed in ice cold acetone at −20° C. for 20 minutes. After fixation, slides were dried briefly at room temperature and stored at −80° C. until stained. For staining, slides were equilibrated at room temperature, washed in PBS twice for five minutes, blocked in serum-free blocking reagent (Dako) ON at 4C, followed by staining with Srebp2 (ab30682, Abcam), CD103 PE (2E7, Invitrogen), CD45.2 FITC (104, Invitrogen) diluted in Antibody diluent solution (Dako) ON at 4 C, followed by a 2 h incubation with Donkey Anti-Rabbit AF647 (Thermo Fisher), stained with DAPI, and mounted with coverslips using VECTASHIELD™ hardset mounting media. Images were acquired on a LSM700™ (Zeiss) confocal microscope and APERIO SCANSOPE™ at 20× and 63× resolution. Deidentified human Formalin-Fixed Paraffin-Embedded (FFPE) samples from healthy subjects were acquired from NovusBio. Slides were treated according to standard immunohistochemistry procedures and stained with anti-HMGCR (LS-B16059, LSBio), anti-CD3e (ab11089, Abcam), and anti-CD8a (66868, Thermo Fisher), followed by secondaries anti-rabbit AF647, anti-mouse AF488, and anti-rat AF594. Images were acquired at 20× with an Aperio FL fluorescence scanner system. Cell segmentation and quantification of HMGCR expression was done using QUPATH™ 6. In short, DAPI stains were used for cell detection with the following parameters; requestedPixelSizeMicrons 0.2, backgroundRadiusMicrons 2.5, sigmaMicrons 0.5, minAreaMicrons 5.0, maxAreaMicrons 50, threshold 300, waterShedPostProcess True, cellExpansionMicrons 1.5. CD8a cells were identified by training an object classifier based on “Random trees” using CD8α, and CD38 staining, as well as cell morphology features. HMGCR expression was obtained using compute intensity features on the ROI CD8 mask with TILESIZEMICRONS 10.0™

### Ultra-Low Input (ULI) RNA-Sequencing

For studies pertaining to the profiling of shCd19 or shSrebf2 P14 CD8 T cells: for each replicate, cells from 2 mice were pooled and then sorted for ULI RNA-seq following Immunological Genome Project (ImmGen) guidelines. Seven, and 14 days after initial infection with LCMV, 5×10³ Ametrine⁺ P14 cells from each congenic were sorted from the spleen, and 5×10³ iv⁻ Ametrine⁺ P14 cells were sorted from the IEL, kidney, and liver into PBS+5% BSA+2% FBS+0.5 M EDTA. 1×10³ cells were then resorted into 5 μl of 1×TCL lysis buffer+1% 2-mercaptoethanol.

For studies pertaining to the profiling of SI T_{RM} from mice fed low or high cholesterol-containing diets: Mouse previously infected (>30 days) with LCMV and transferred with P14 CD8 T cells were fed a low or high cholesterol diet for 7 days. After preparation of single-cell suspensions, 5×10³ iv⁻ P14 CD8 T cells were sorted from the IEL, into PBS+5% BSA+2% FBS+0.5 M EDTA. 1×10³ cells were then resorted into 5 μl of 1×TCL lysis buffer+1% 2-mercaptoethanol. Library preparation for ULI RNA-seq was performed as described online at Immgen (“ImmGenULI_RNAseq_methods.pdf”). TRIMMOMATIC™ was used to remove adapters and trim low-quality reads (NexteraPE-PE.fa:2:30:10:1 TRUE LEADING:3 TRAILING:3 SLIDINGWINDOW:4:15 MINLEN:25)⁷. Trimmed reads were then aligned to the gencode M25 annotation of the mm10 genome using STAR with the default conditions. Aligned reads were then quantified with FEATURECOUNTS™ (-t exon -g gene id -p -B), and differentially expressed genes were identified using DEseq2⁸. GSVA was performed using the Gene set variation analysis (GSVA) package in R⁹. When raw expression counts were used as input, kcdf was set to Poisson, otherwise kcdf was set to Gaussian.

### CRISPR-Cas9 Mutagenesis Screening Using the Lentiviral Metabolic Library

Lentiviral sgRNA metabolic library has been previously published^{10,11}. In short, this custom mouse metabolic library contains 3,017 genes synthesized based on the gene list from reported human metabolic genes. A total of 6 gRNAs were designed for each gene according to previously published criteria. Two sub-libraries (Lib5 and Lib7) were made, with each containing 3 gRNAs targeting one gene and 500 non-targeting controls. The synthesis, purification, and quality control of the library was described previously. The in vivo screening approach was modified from previous studies. In short, lentivirus was produced by co-transfecting the lentiviral metabolic library plasmids, psPAX2, and pCAG4-Eco in HEK293T cells. At 48 h after transfection, the supernatant was harvested and frozen at −80° C. Naive P14 Cas9eGFP+ were isolated and pooled from lymph nodes and spleens from 8 P14 Cas9eGFP mice followed by in vitro activation with plate-bound anti-CD3 (1 g/ml; 145-2C11; eBioscience) and anti-CD28 (1 g/ml; 37.51; eBioscience) antibodies for 24 hours. Immediately after, cells were spin infected by centrifugation with Lib5 and Lib7 library supernatants supplemented with 1:200 LentiBoost (Sirion Biotech) and 55 μM beta-mercaptoethanol for 3 h at 800 G, followed by 4 h incubation at 37° C. 5% CO₂. Immediately after, cells were washed and cultured with human IL-2 (20 IU/ml; Peprotech), mouse IL-7 (2.5 ng/ml; Peprotech) and IL-15 (25 ng/ml; Peprotech) for 4 days to expand and allow gene editing to occur. Next, cells were sorted based on the expression of Ametrine, and an aliquot of 5×10⁶ transduced Cas9eGFP P14 cells was saved as “input” (around 500×cell coverage per sgRNA). Transduced P14 cells (5×10⁵) were then transferred IV to 7, and 9 naive C57BL/6 mice for Lib5 and Lib7, respectively, followed by LCMV infection (2×10⁵ PFU) 1 h later. At day 13 and day 14 after LCMV infection for Lib 5 and Lib7, respectively, donor-derived T_{CM} (CD62L⁺CD127⁺), T_{EM}, and t-T_{EM} from the spleen, T_{EM} and T_{RM} from the liver, and T_{RM} from the SI were sorted into FBS and frozen at −80° C. until genomic DNA extraction. Cell coverage per sgRNA per population and library were: Lib5: T_{EM}=110, T_{CM}=16, t-T_{EM}=66, Liver T_{EM}=28, Liver T_{RM}=8, SI T_{RM}=12, and Lib7: T_{EM}=141, T_{CM}=30, t-T_{EM}=77, Liver T_{EM}=50, Liver T_{RM}=17, Gut T_{RM}=31.

### Targeted CRISPR Cas9 Screen

sgRNAs targeting the 50 selected genes of the mevalonate and cholesterol synthesis pathway and adjacent enzymes were picked from either CRISPICK™ (CRISPiCK) (Broad Institute) or from the 3017 metabolic library and ordered as primers with BbsI overhangs from IDT as OPOOLS OLIGO POOLS™ (IDT) and cloned into BbsI-linearized LsgA vector. Library preparation and equal sgRNA representation verification was done by subcloning into Stbl3 bacteria and sequencing of individual clones. Retroviral supernatant was titrated to achieve a targeted transduction efficiency of 30%, which is equivalent to an MOI of ~0.3. sgCd19 and sgFdft1 sgRNA were cloned into LMG retroviral plasmid. Double transduction of either LMG-sgCd19 or LMG-sgFdft1 with the targeted LsgA library was done via spinoculation of enriched and in vitro activated P14 Cas9eGFP T cells following by 24 h expansion in hIl-2 (10 u/mL). LMG-derived eGFP expression is two orders of magnitude stronger than eGFP on P14 T cells, thus making the identification of LMG-transduced P14 Cas9eGFP cells possible by flow cytometry. After sorting double positive (GFP⁺ Ame⁺) transduced P14 T cells, 5×10⁵ cells from either sgCd19 and sgFdft1 groups were transferred into 6 male B6 mice each and infected with LCMV 1 hour later. Cell pellets from double-transduced sgCd19 and sgFdft1 were kept as input. T_{E} and T_{MP} populations were sorted based on CD127 and KLRG1 stain from blood pooled from 6 mice of each condition at day 7 after infection and cell pellets were kept at −80 C. Spleen T_{EM} and liver T_{RM} populations were sorted at day 14 after LCMV infection based on the expression of CD127⁺ and CD62L⁻ stain (T_{EM}), and IV⁻ stain (T_{RM}) of total P14 in the spleen, and liver, respectively, and pellets were stored at −80° C. until processing. The SI T_{RM} population included LP and IEL fractions and were similarly sorted at day 14 after LCMV infection by FACS. The dLN and TIL populations were sorted from single cell preparations from 5 pooled dLN and 5 pooled MC38-GP₃₃₋₄₁ tumors 6 days after adoptive cell transfer. The cell yields and cell per sgRNA coverage for each sample were as follows: Input sgCd19, 1×10⁵ total cells at 645 cells/sgRNA, Input sgFdft1, 1×10⁵ total cells at 645 cells/sgRNA, sgCd19 T_{E}, 2.39×10⁵ total cells at 1542 cells/sgRNA, sgCd19 T_{MP}, 1×10⁴ total cells at 65 cells/sgRNA, sgCd19 Liver T_{RM}, 1×10⁴ total cells at 65 cells/sgRNA, sgCd19 Sp T_{EM}, 1×10⁴ total cells at 65 cells/sgRNA, sgFdft1 T_{E}, 1.6×10⁵ total cells at 1032 cells/sgRNA, sg Fdft1 T_{MP}, 1×10⁴ total cells at 65 cells/sgRNA, sgFdft1 Liver T_{RM}, 6×10³ total cells at 39 cells/sgRNA, and sgFdft1 Sp T_{EM}: 2.5×10³ at 16 cells/sgRNA. The cell yields and cell per sgRNA coverage of the SI T_{RM} targeted screen were as follows: Input, 1×10⁶ cells at 645 cells/sgRNA, SI T_{RM}, 4.8×10³ cells at 31 cells/sgRNA. The cell yields and cell per sgRNA coverage of the dLN and TIL screen in MC38-GP₃₃₋₄₁ were as follows: Input, 5×10⁴ total cells at 323 cells/sgRNA, dLN, 3×10³ total cells at 19 cells/sgRNA. TIL fractions were collected in two subfractions: Tim3^{low}, 4,425 cells at 29 cells/sgRNA, and Tim3^{high}, 3,065 total cells at 20 cells/sgRNA. Frequency of Tim3^{high} and Tim3^{low} was ~50%. Fastq files from these two fractions were later combined to simplify analysis and increase coverage for TIL fraction. Genomic DNA was extracted according to the manufacturer's instructions (DNeasy, Qiagen), Qiagen). The sgRNA-containing LsgA cassette was amplified with LV primers forward 5′ TTTCTTGGCTTTATATATCT 3′ and reverse 5′ GACTAGCCTTATTTTAACTT 3′. The PCR product was cleaned according to the manufacturer's instructions (QIAquick PCR purification), and Nextera handles were added by PCR using Nextera Handle forward 5′-TCGTCGGCAGCGTCAGATGTGTATAAGAGACAGTTTCTTGGCTTTATATAT CT 3′ (SEQ ID NO:9) and reverse 5′ GTCTCGTGGGCTCGGAGATGTGTATAAGAGACAGGACTAGCCTTATTTTA ACTT 3′ (SEQ ID NO:10). After PCR product purification, PCR product was indexed with Illumina compatible indices. All PCR steps were done using proof-reading DNA polymerase Q5 (NEB). Library sizes were verified in the high sensitivity D1000 screen tape system (TapeStation, Agilent). Sequencing was performed in a Novaseq in a PE100 run format. sgRNA frequencies were obtained using Mageck¹² count using the read 1 Fastq file and statistical testing against a background of six non-targeting sgRNAs using Mageck-test of each sample output to its corresponding input. Only the verified sgRNA for Srebf2 (Lib5_20788.3: ACTCCAGTGACAGTACACTG (SEQ ID NO:11)) was used for analysis.

### Adoptive Therapy Tumor Model

For adoptive therapy experiments, 5×10⁵ B16-GP₃₃₋₄₁ cells or MC38-GP₃₃₋₄₁, which were verified Mycoplasma negative by PCR, and authenticated by in vitro killing assays, were transplanted subcutaneously into the right flank of wild-type B6 mice. After tumors became palpable, 7-8 days after transplantation, in vitro activated and expanded P14 CD8 T cells were transferred intravenously. For comparison of TIL accumulation in a mixed transfer setting, naive P14 CD8 T cells were activated, transduced and expanded with 50 U/mL of hIL-2 for 2-3 days; cells transduced with control constructs (Cd19 shRNAmir) or experimental constructs (Srebf2 shRNAmir) were mixed 1:1 and 1×10⁶ P14 cells were transferred intravenously. For efficacy studies, transduced cells were expanded for 5-6 days; transduced cells were then sorted, and 1×10⁶-2.5×10⁶ cells were transferred intravenously into mice with established MC38-GP₃₃₋₄₁ or B16-GP₃₃₋₄₁ tumors. Tumors were monitored daily, and mice with ulcerated tumors or tumors exceeding 400 mm² were euthanized, in accordance with Univ. Calif. San Diego (UCSD) Institutional Animal Care and Use Committee (IACUC).

### Zaragozic Acid A (ZAA), Anti-PD1 Treatment, and CD8 Depletion in Mouse Tumor Models

At day 6 after tumor injection, the mice were randomized and treated every other day with ZAA (Santa Cruz Biotechnology) or vehicle control by alternating intravenous (200 pg/mouse) and intraperitoneal (100 pg/mouse) injections until day 16. For combination therapy with anti-PD1 (programmed cell death protein 1 (PD-1)) antibody, ZAA was administered with the same schedule, and three consecutive treatments of anti-PD1 (InVivoMab anti-mouse PD-1 (CD279), BioXCell, BE0033-2) at 200 μg/mouse were delivered IP on days 9, 10, and 11 after tumor injection. Isotype control for Syrian IgG was used (InVivoMAb polyclonal Syrian hamster IgG, BioXCell). For CD8-depleting experiments in combination with ZAA, anti-CD8a Ab (2.43 BioXCell) was administered intraperitoneally at a dose of 200 μg/mouse starting 2 days prior to tumor implantation for three consecutive days (−2, −1, 0), followed by 2 additional doses of 100 μg/mouse on days 4 and 8, and ZAA treatment scheduled was started at day 4 after tumor implantation. Isotype control for Rat IgG2 was used (InVivoMAb rat IgG2a isotype control, anti-trinitrophenol, BioXCell).

### Lovastatin Treatment

2 days after LCMV Arm infection, mice were randomized into two groups and treated with either Vehicle (DMSO) or 10 mg/kg of Lovastatin (Calbiochem, 438185) in DMSO via IP injection daily for 5 consecutive days, followed by treatment once every three days after day 7 after LCMV infection until day 21 after LCMV infection.

Cloning of Mouse Pdss2 CCDS in the pMIA Vector

Total cDNA from liver homogenates of B6 WT mouse was used to PCR amplify Pdss2 CCDS by using the primers F (5′-3′): TCTGAGGGTTTGGAATCGCC (SEQ ID NO:12), and R (5′-3′): CCGCCTCATTCTCTGGGTTT (SEQ ID NO:13) using a touch-down protocol starting at 62 with −0.5 C delta for 10 rounds, followed by 25 rounds of amplification at 58 C. PCR product was cleaned and reamplified with primers F (5′-3′): TAGCGGATCCCAATTGCTCGAGgccaccATGAGCCTCCGGCAGCTGCTGTTG CGCTTG (SEQ ID NO:14) and R (5′-3′): GGGCGGAATTGATCCCGCTCGAGTCAAGAAAATCTGGTCACAGCAAAC (SEQ ID NO:15). This second set of primers adds complementary overhangs to the pMIA vector to allow cloning into Xho-I linearized pMIA plasmid using the NEBuilder HiFi DNA Assembly standard protocol (NEB), and add the Kozak sequence (gccacc) to promote gene expression. Cloned Pdss2 gene sequence was verified by full plasmid sequencing (Primordium).

shRNAmir Sequences and sgRNA Cloning in LsgA and LMG Vectors

The following sequence was used for the shRNAmir targeting of Srebf2 with the pLMPd-Amt retroviral construct: 5′-TGCTGTTGACAGTGAGCGATGCTCTAGAGTATTTGAAATATAGTGAAGCC ACAGATGTATATTTCAAATACTCTAGAGCACTGCCTACTGCCTCGGA-3′ (SEQ ID NO:16), and Cd19 (control): 5′-TGCTGTTGACAGTGAGCGAATGGATAAGTCTGACGACCTATAGTGAAGCC ACAGATGTATAGGTCGTCAGACTTATCCATGTGCCTACTGCCTCGGA (SEQ ID NO:17). Non-targeting (NT) shRNA (TRNSU6001), and shRNAmir targeting Fdft1 (ULTRA-3215720) were purchased from Transomics. The following primer pairs used for CRISPR/Cas9-mediated gene disruption were hybridized by heating at 95° C. for 5 minutes followed by a ramp down of 1° C. each 30 s to 4° C. in UltraPure Distilled Water (Invitrogen):

sgCd19 forward
(SEQ ID NO: 18)
5′-CACCGACCTTCACGTGCCTCTCGA
sgCd19 reverse
(SEQ ID NO: 19)
5′-AAACTCGAGAGGCACGTGAAGGTC-3′
sgSrebf2 (Lib5-20788.3) forward
(SEQ ID NO: 20)
5′-CACCGACTCCAGTGACAGTACACTG-3′
sgSrebf2 (Lib5-20788.3) reverse
(SEQ ID NO: 21)
5′-AAACCAGTGTACTGTCACTGGAGTC-3′
sgFdft1 (Lib7-14137.1) forward
(SEQ ID NO: 22)
5′-CACCCCATCCCACACCCCATCCGG-3′
sgFdft1 (Lib7-14137.1) reverse
(SEQ ID NO: 23)
5′-AAACCCGGATGGGGTGTGGGATGG-3′
sgHpd (Lib5-15445.3) forward
(SEQ ID NO: 24)
5′-CACCTGGAGATACCACACACACCC-3′
sgHpd (Lib5-15445.3) reverse
(SEQ ID NO: 25)
5′-AAACGGGTGTGTGTGGTATCTCCA-3′
sgPdss2 (Lib5-71365.1) forward
(SEQ ID NO: 267)
5′-AAACATGATATTGGAATCTCGACC-3′
sgPdss2 (Lib5-71365.1) reverse
(SEQ ID NO: 27)
5′-AAACGGTCGAGATTCCAATATCAT-3′

Hybridized primer pairs were cloned into BbsI-linearized LsgA or LMG constructs by overnight ligation (T4 DNA Ligase, NEB) at room temperature, following transformation into DH5α (ThermoFisher). In some instances, a “G” was added at the beginning of sgRNA sequences not naturally starting with a G to increase transcription efficiency.

qPCR and Sanger Sequencing Validation of Gene Targeting

For validation of Srebf2, Fdft1, Pdss2 and Hpd gene targeting efficiencies with shRNAmir and sgRNA retroviral constructs, enriched CD8 T cells were activated, transduced, and expanded for 4-6 d in 25 mg/ml hIL-2 (Cetus). Ametrine⁺ cells (Srebf2 shRNAmir, Srebf2 sgRNA, Hpd sgRNA, Pdss2 sgRNA, Fdft1 sgRNA, Fdft1 shRNA, non-targeting shRNA, or control Cd19 shRNAmir and sgRNA) were sorted directly into TRIzol (Life Technologies) for RNA quantification of shRNAmir knockdown, or MACS buffer with 2% FBS for genomic DNA isolation. RNA, and DNA were extracted per manufacturer specifications (RNeasy, Qiagen for RNA, DNeasy, Qiagen for genomic DNA). cDNA was then synthesized using Superscript II (Life Technologies) and quantitative PCR was performed using the Stratagene Brilliant II Syber Green master mix (Agilent Technologies). Srebf2 and Hmgcr expression levels were normalized to the housekeeping genes Hprt1. The following primers were used for qPCR:

Srebf2 forward:
(SEQ ID NO: 28)
5′-GCGTTCTGGAGACCATGGA-3′
Srebf2 reverse
(SEQ ID NO: 29)
5′-ACAAAGTTGCTCTGAAAACAAATCA-3′
Hmgcr forward
(SEQ ID NO: 30)
5′-CTCGTGGAATGCCTTGTGATTG-3′
Hmgcr reverse
(SEQ ID NO: 31)
5′-AGCCGAAGCAGCACATGAT-3′
Hmgcs1 forward
(SEQ ID NO: 32)
5′-GCCGTGAACTGGGTCGAA-3′
Hmgcs1 reverse
(SEQ ID NO: 33)
5′-GCATATATAGCAATGTCTCCTGCAA-3′
Ldlr forward
(SEQ ID NO: 34)
5′-AGGCTGTGGGCTCCATAGG-3′
Ldlr reverse
(SEQ ID NO: 35)
5′-TGCGGTCCAGGGTCATCT-3′
Hprt1 forward
(SEQ ID NO: 36)
5′-TGAAGAGCTACTGTAATGATCAGTCAAC-3′
Hprt1 reverse:
(SEQ ID NO: 37)
5′-AGCAAGCTTGCAACCTTAACCA-3′
Pdss2 forward:
(SEQ ID NO: 38)
5′-CACCATGATATTGGAATCTCGACC-3′
Pdss2 reverse:
(SEQ ID NO: 39)
5′-AAACGGTCGAGATTCCAATATCAT-3′
Fdft1 forward:
(SEQ ID NO: 40)
5′-GGATGTGACCTCCAAACAGGAC-3′
Fdft1 reverse:
(SEQ ID NO: 41)
5′-CAGACCCATTGAGTTGGCACAC-3′

Targeting efficiency of sgSrebf2, sgFdft1, sgPdss2, and sgHpd constructs on genomic DNA was assessed by PCR amplification of 100 ng of genomic DNA using a high-fidelity polymerase (Q5 HIGH FIDELITY™ DNA polymerase 2×, New England Biolabs) using a touchdown protocol (63° C. Tm −0.5 C/cycle for 10 cycles, followed 25 by 25 cycles at 58 C). PCR products were purified according to the manufacturer's instructions (QIAQUICK™ PCR purification, Qiagen). Genomic DNA was amplified with the following primers:

Srebf2 forward
(SEQ ID NO: 42)
5′-ATCATGGCTGTTCTGACTCGG-3′
Srebf2 reverse
(SEQ ID NO: 43)
5′-GAAGCCCATTTTTGCACAGGG-3′
Fdft1 forward
(SEQ ID NO: 44)
5′-CTTCATGGGTCCTTGGGGAAC-3′
Fdft1 reverse
(SEQ ID NO: 45)
5′-CCTGTGGGGCGTGTATTGTCA-3′
Hpd forward
(SEQ ID NO: 46)
5′-GCATCACAGTCTCCCATCCT-3′
Hpd reverse
(SEQ ID NO: 47)
5′-CTGTCTCACCTCCCGAAGTTT-3′
Pdss2 forward
(SEQ ID NO: 48)
5′-TTGTCCCCTGCTACCATGTTC-3′
Pdss2 reverse
(SEQ ID NO: 49)
5′-GAATGCCATGTCTTGGACCG-3′

Sanger sequencing of purified PCR products (Eton Biosciences) was done using the following primers:

Fdft1
(SEQ ID NO: 50)
5′-AACCATGAAGTGTGTCAT-3′
Srebf2
(SEQ ID NO: 51)
5′-AGCTGCTTTTCTGAAGGT
Hpd
(SEQ ID NO: 52)
5′-ACTACCGAGCAAAACGGCAG-3′
Pdss2
(SEQ ID NO: 53)
5′-GCCCTGTCTTCATGATGCCC-3′

Genome editing by CRISPR/Cas9 for each construct was calculated with TIDE¹³.

### Curation of Metabolic Signatures

We constructed a list of minimally overlapping metabolic gene sets (Metab205) by aggregating custom gene lists, and all gene sets from KEGG™, BIOCARTA™, and REACTOME™ listed in the C2.CP.gmt file from MSIGDB™ (Broad Institute), and iterating rounds of curation using the GSVA tool computeGeneSetsOverlap in R. To use Metab205 on mouse data sets, bulk microarray and RNAseq expression data sets were converted to human gene symbols using the Gene Set Enrichment Analysis (GSEA) Collapse Dataset tool with Mouse_Gene_Symbol_Remapping_Human_Orthologs_MSigDb.chip file with default options. For MAGECK analysis of pathway enrichments, and single cell RNAseq analysis, human Metab205 gene symbols were translated to mouse gene symbols using bioDBnet.

scRNAseq of P14 in the Subcutaneous MC38-GP₃₃₋₄₁ Model

P14 from tumors and spleens of mice were profiled by scRNAseq in two independent repeats. For each repeat, 2×10⁵ P14 cells coming from pooled spleens and MC38-GP₃₃₋₄₁ tumors from three independent mice were sorted into T cell culture media. Samples were spun down at 500 rcf for 5 minutes and resuspended in PBS+0.04% (w/v) bovine serum albumin. Samples were then loaded into CHROMIUM CHIP B™ (10× Genomics) and partitioned into Gel Bead In-Emulsions (GEMs) in a chromium controller (10× Genomics). Single cell RNA libraries were generated according to the Chromium Single Cell 3′ Reagent Kits v3 User Guide and sequenced on a HISEQ 4000™. Reads were aligned to the mm10 genome using CELLRANGER™ count and final data matrices for all samples were generated with CELLRANGER™ aggregate. The resulting counts matrix was then processed using Seurat in RSTUDIO™ and cells with mitochondrial read % greater than 7 were discarded. Data was normalized and scaled using NORMALIZEDATA™ and SCALEDATA™ followed by batch correction of biological replicates was corrected with Harmony¹⁴. The top 2000 most variable genes were calculated using FINDVARIABLEGENES™ and used then used in the PCA calculation using RUNPCA™ (principal components analysis (PCA)). After adding Harmony embeddings, we applied dimensionality reduction by uMAP using harmony reduction, and 1:20 dimensions. Louvain clustering was performed with Seurat's FINDCLUSTERS™ based on the top 20 principal components with the resolution set to 0.5. Final gene expression values were normalized using sctransform in SEURAT™. Additionally, data imputation was performed using MAGIC™¹⁵ with the log-normalized expression values and the default settings and the exact solver. Seurat's ADDMODULESCORE™ with default settings function was used to calculate scores for the mevalonate/cholesterol synthesis pathway gene list. Heatmap was generated by extracting average SCT gene expression values for each group, scaled gene expression values by row, and plotted with COMPLEXHEATMAP™¹⁶, installed from Bioconductor, with column clustering.

### Bioinformatic Analysis of Publicly Available Single-Cell RNA-Seg Datasets

Human single cell RNAseg of immune populations from PBMC, SI (Ileum) and colon of healthy patients: Data were downloaded from GEO (GSE125527). Count matrices for each sample were acquired using CELLRANGER™ “count” followed by CELLRANGER™ aggregation to obtain a pooled data set. Filtered_feature_bc_matrix files were loaded into Seurat in RSTUDIO™ to construct the Seurat object. Cells with nCount between 500 and 15000, and less than 25% mitochondrial reads were retained. Gene expression was normalized with SCTRANSFORM™, followed by standard Seurat clustering and dimensionality reduction steps with 40 dimensions and 0.8 resolution. CD8 T cells from healthy subjects were further subset using SingleR¹⁷. Imputation of gene expression was performed using MAGIC¹⁵. Core T_{RM} score¹⁸ were calculated with UCELL™ ¹⁹ in CD8 T cells from the spleen and the SI, and values were tested for statistical significance between statin and non-statin users for each tissue using the ggsignif package. The inclusion criteria for statin use included subjects who were actively taking statin medication at least two weeks before sampling.

Pan-cancer single-cell RNAseg atlas of tumor-infiltrating CD8 T cells²⁰: integrated SingleCellExperimnt.rds file (int.CD8.S35.sce.merged.rds) was retrieved from the Zenodo repository (10.5281/zenodo.5461803) and transformed to a Seurat Object in R. Mevalonate/cholesterol synthesis pathway scores were calculated using ADDMODULESCORE™. The heatmap was generated with ComplexHeatmap with Euclidean distances to cluster rows (cancer type) and columns (meta.cluster.coarse). DIMPLOT™ was used to plot umap.harmony embeddings in a 2D plot grouped by “meta.cluster.coarse” annotations. Data from the CELLTYPIST™ resource was downloaded from (https://www.tissueimmunecellatlas.org/) as an h5ad file, converted to a Seurat object, and mevalonate/cholesterol signature scores were calculated with UCELL™. DOTPLOT™ using the original metadata were used to plot scores grouped by different categories. The human signature for the mevalonate cholesterol synthesis pathway used was: ACAT1, ACAT2, CYP51A1, EBP, FDFT1, FDPS, GGPS1, HMGCR, HMGCS1, HMGCS2, IDIJ, LBR, LSS, MVD, MVK, PMVK, TM7SF2, DHCR7, DHCR24, NSDHL, HSD17B7, MSMO1, SC5DL, LOC651621, SQLE, LOC730412, IDI2. (Note; The SC5DL gene name was updated to SC5D on Jun. 1, 2020.)

### In Vivo Bodipy-Cholesterol, Bodipy-LDL Uptake Assays, and Serum Total Cholesterol Measurements

200 μl of a 10 mg/ml of Bodipy-Cholesterol in corn oil (Sigma) solution (2 mg Bodipy-Cholesterol) was orally gavaged into C57B6 mice. After 16 minutes, mice were iv labeled with CD8α, antibody, and 4 minutes later mice were taken down for immune cell profiling by flow cytometry. Bodipy-Cholesterol incorporation into spleen CD8 T cells was used as a negative control. One mouse per experiment was orally gavaged with 200 ul of corn oil to establish baseline signal. 200 μl of 1 mg/ml of Human Bodipy-LDL (Invitrogen) was delivered IV by RO injection. After 26 minutes, mice were iv labeled with 3 μg of CD8a antibody, and 4 minutes later mice were taken down for immune cell profiling by flow cytometry. One mouse per experiment was RO injected with PBS to establish baseline signal. Cholesterol measurement in mouse serum was done using the AMPLEX RED CHOLESTEROL ASSAY™ (Thermo Fischer) according to the manufacturer's instructions. Mouse serum was collected by submandibular bleeding followed by centrifugation at 7000 rcf for 15 minutes at 4° C.

Intracellular Metabolite Isolation with Liquid Chromatography-Tandem Mass Spectrometry (LC-MS/MS) and Analysis of Metabolomics Data.

To assess relative steady-state variation between metabolic intermediates in tissue resident and peripheral T cell populations, freshly sorted populations were washed twice with ice-cold phosphate-buffered saline, centrifuged at low speed to avoid lysis, and stored without buffer at −80° C. prior to analysis. Intracellular metabolite isolates were prepared as previously described²¹. Briefly, pelleted cells were lysed in a cold solution of 4:1 methanol:water and rapidly cycled between −80° C. for one minute and 37° C. for one minute, three times. Lysates were centrifuged at 14,000 RPM for 10 minutes at 4° C. to separate soluble metabolite extract from insoluble cell debris. Extracts were dried by vacuum concentrator and resuspended in a solution of cold 2:2:1 acetonitrile:methanol:water at 20,000 cell equivalents per microliter, and transferred to glass liquid chromatography vials for analysis. All solvents used were HPLC-grade (Fisher Scientific). Identification and quantification of extracted metabolites by LC-MS/MS analysis was described previously²². Sampling and liquid chromatography was performed on a VANQUISH™ Ultra High Performance Liquid Chromatograph (UHPLC) system (Thermo Scientific) with SEQUANT ZIC-PHILIC™ polymeric column chromatography (100 mm×2.1 mm, 5 μm) (EMD Millipore), maintained at 45° C. Cell extracts were separated by autosampler injection of 2 μL sample and eluting on a constant 0.4 mL/minute fluid flow rate. Elution followed a linear flow gradient of: (A) 20 mM ammonium bicarbonate in water, pH 9.6, and (B) acetonitrile; 90% B for 0.25 minutes, a linear gradient to 55% B at 4 minutes, and maintained at 55% B until 6 minutes. Solid phase chromatography was then re-equilibrated for 2.5 minutes at 90% B between samples. Metabolite detection was performed via a coupled QEXACTIVE HYBRID QUADRUPOLE-ORBITRAP MASS SPECTROMETER™ (Thermo Scientific). Samples were eluted in duplicate for acquisition in positive and negative ionization mode. Ionization was by a heated electrospray ionization (HESI) source: spray voltage of 3.5 kV; auxiliary gas, sheath gas, and sweep gas flow rates of 20, 40, and 2 AU, respectively. Capillary gas heater temperature was 275° C. and auxiliary gas heater temperature was 350° C. MS1 profile spectra were acquired in a mass range of 67-1000 m/z, with resolution of 35,000, AGC volume of 1×10⁶ maximum IT of 75 ms. Data-dependent tandem mass spectra were acquired to aid in metabolite identification, using a normalized collision energy of 25 eV, an isolation window of 1.5 m/z, resolution of 17,500, a loop count of 5, AGC volume of 1×10⁵, and with maximum IT of 50 ms. Metabolite identifications were made against an in-house library of standards or against tandem spectral matching from publicly available databases housed in the Global Natural Product Social Molecular Networking (GNPS) Library (gnps.ucsd.edu) and meeting criteria for a match score of >0.70 and accurate mass window of 10 ppm. Tandem mass spectra matching against commercial standards of 4-hydroxybenzaldehyde (Millipore Sigma, 91554) and mevalonate (Millipore Sigma, 42147) were derived from the most abundant ion for each compound: m/z=121.0295 for 4-hydroxybenzaldehyde [M-H], and m/z=147.0663 for mevalonate [M-H]. Black spectra are derived from cell lysate, and red mirrored spectra from HPLC and GC-grade commercial standards. Intensity matrices for identified metabolites, or identified and annotated metabolites, were processed using METABOANALYST 5.0™ to perform clustered heatmaps and PCA.

### Quantification of CoQ Species in CD8 T Cells

Enriched, in vitro-activated CD8 T cells with CD3/CD28, as previously described, were expanded in 50 U/mL of human IL-2 for 24 and treated with ZAA at 1 μM for 48 h. Similarly, in-vitro-activated CD8 T cells were transduced with LsgA-sgCd19, LsgA-sgFdft1, pMIA-Empty, or pMIA-Pdss2 constructs and expanded 48 h in 50 U/mL of human IL-2 before sorting 1-2×10⁶ Ame⁺ cells per sample. 1-2×10⁶ cells were pelleted and frozen at −80° C. until processing. Lipids were extracted using a modified version of the Bligh-Dyer method. Briefly, frozen cell pellets were resuspended in 350 μL of MILLIQ™ water shaken in an Eppendorf tube with 300 μL methanol and 1 mL chloroform containing internal standards (d7-Cholesterol and d6-CoQ10) for 30 s. The resulting mixture was vortexed for 15 s and centrifuged at 2600×g for 8 minutes to induce phase separation. The organic (bottom) layer was retrieved using a Pasteur pipette, dried under a gentle stream of nitrogen, and reconstituted in 2:1 chloroform:methanol for LC/MS analysis. Lipidomic analysis was performed on a Vanquish HPLC online with a Q-Exactive quadrupole-orbitrap mass spectrometer equipped with an electrospray ion source (Thermo). Data was acquired in positive ionization mode. Solvent A consisted of 95:5 water:methanol, Solvent B was 70:25:5 isopropanol:methanol:water. Solvents A and B contained 5 mM ammonium formate with 0.1% formic acid. An XBRIDGE™ (Waters) C8 column (5 m, 4.6 mm×50 mm) was used for separation. The gradient was held at 0% B between 0 and 5 min, raised to 20% B at 5.1 min, increased linearly from 20% to 100% B between 5.1 and 55 min, held at 100% B between 55 minutes and 63 min, returned to 0% B at 63.1 min, and held at 0% B until 70 min. Flow rate was 0.1 mL/minute from 0 to 5 min, 0.3 mL/minute between 5.1 minutes and 55 minutes, and 0.4 mL/minute between 55 minute and 70 min. Spray voltage was 3.5 kV; S-lens RF level is 65; Sheath, auxiliary, and sweep gases were 50, 10 and 1, respectively. Capillary temperature was 325° C. and auxiliary gas heater temperature was 200° C. Data was collected in full MS/dd-MS2 (top 10). Full MS was acquired from 150-1500 m/z with resolution of 70,000, AGC target of 1×10⁶ and a maximum injection time of 100 ms. MS2 was acquired with a resolution of 17,500, a fixed first mass of 50 m/z, AGC target of 1×10⁵ and a maximum injection time of 200 ms. Stepped normalized collision energies were 20, 30 and 40%. SKYLINE™ 2³ was used to measure peak areas of a list of lipids of interest (CoQ9, CoQ10, DMQ9, DMQ10). Normalized peak areas were used in data reporting. Data were normalized using internal standards and total protein quantification.

### REFERENCES

1. Masopust, D. et al. Tissue-Resident T Cells and Other Resident Leukocytes. Annu Rev Immunol (2019) doi:10.1 146/annurev-immunol-042617-053214.
2. Park, S. L., et al. Tissue-Resident Memory T Cells in Cancer Immunosurveillance. Trends Immunol 40, 735-747 (2019).
3. Byrne, A. et al. Tissue-resident memory T cells in breast cancer control and immunotherapy responses. Nature Reviews Clinical Oncology Preprint at https://doi.org/10.1038/s41571-020-0333-y (2020).
4. Konjar, S. et al. Mitochondria maintain controlled activation state of epithelial-resident T lymphocytes. Sci Immunol 3, 2543 (2018).
5. Steinert, E. M. et al. Quantifying Memory CD8 T Cells Reveals Regionalization of Immunosurveillance. Cell 161, 737-749 (2015).
6. Wijeyesinghe, S. et al. Expansible residence decentralizes immune homeostasis. Nature 592, 457-462 (2021).
7. Soerens, A. G. et al. Functional T cells are capable of supernumerary cell division and longevity. |Nature|614, (2023).
8. Gebhardt, T. et al. Memory T cells in nonlymphoid tissue that provide enhanced local immunity during infection with herpes simplex virus. Nature Immunology 2009 10:5 10, 524-530 (2009).
9. Iijima, N. & Iwasaki, A. A local macrophage chemokine network sustains protective tissue-resident memory CD4 T cells. Science (1979) 346, 93-98 (2014).
10. Jiang, X. et al. Skin infection generates non-migratory memory CD8+ T_{RM} cells providing global skin immunity. Nature 2012 483:7388 483, 227-231 (2012).
11. Teijaro, J. R. et al. Cutting Edge: Tissue-Retentive Lung Memory CD4 T Cells Mediate Optimal Protection to Respiratory Virus Infection. The Journal of Immunology 187, 5510-5514 (2011).
12. Ariotti, S. et al. Skin-resident memory CD8+ T cells trigger a state of tissue-wide pathogen alert. Science (1979) 346, 101-105 (2014).
13. Schenkel, J. M. et al. Resident memory CD8 T cells trigger protective innate and adaptive immune responses. Science (1979) 346, 98-101 (2014).
14. Mackay, L. K. et al. Long-lived epithelial immunity by tissue-resident memory T (TRM) cells in the absence of persisting local antigen presentation. Proc Natl Acad Sci USA 109, 7037-7042 (2012).
15. Shin, H. & Iwasaki, A. A vaccine strategy that protects against genital herpes by establishing local memory T cells. Nature 2012 491:7424 491, 463-467 (2012).
16. Wu, T. et al. Lung-resident memory CD8 T cells (TRM) are indispensable for optimal cross-protection against pulmonary virus infection. J Leukoc Biol 95, 215-224 (2014).
17. Milner, J. J. et al. Runx3 programs CD8+ T cell residency in non-lymphoid tissues and tumours. Nature 552, 253-257 (2017).
18. Nizard, M. et al. Induction of resident memory T cells enhances the efficacy of cancer vaccine. Nature Communications 20178:18, 1-11 (2017).
19. Malik, B. T. et al. Resident memory T cells in the skin mediate durable immunity to melanoma. Sci Immunol 2, 6346 (2017).
20. Ganesan, A. P. et al. Tissue-resident memory features are linked to the magnitude of cytotoxic T cell responses in human lung cancer. Nature Immunology 2017 18:8 18, 940-950 (2017).
21. Djenidi, F. et al. CD8+ CD103+ Tumor-Infiltrating Lymphocytes Are Tumor-Specific Tissue-Resident Memory T Cells and a Prognostic Factor for Survival in Lung Cancer Patients. The Journal of Immunology 194, 3475-3486 (2015).
22. Reina-Campos, M., Scharping, N. E. & Goldrath, A. W. CD8+ T cell metabolism in infection and cancer. Nat Rev Immunol 21, (2021).
23. Huang, H. et al. In vivo CRISPR screening reveals nutrient signaling processes underpinning CD8+ T cell fate decisions. Cell 184, 1245-1261.e21 (2021).
24. Crowl, J. et al. Tissue-resident memory CD8+ T cells possess unique transcriptional, epigenetic and functional adaptations to different tissue environments. Nat Immunol (2022).
25. Pan, Y. et al. Survival of tissue-resident memory T cells requires exogenous lipid uptake and metabolism. Nature 543, 252-256 (2017).
26. Milner, J. J. & Goldrath, A. W. Transcriptional programming of tissue-resident memory CD8+ T cells. Curr Opin Immunol 51, 162-169 (2018).
27. Frizzell, H. et al. Organ-specific isoform selection of fatty acid-binding proteins in tissue-resident lymphocytes. Sci Immunol 5, 9283 (2020).
28. Wei, J. et al. Targeting REGNASE-1 programs long-lived effector T cells for cancer therapy. Nature 576, 471-476 (2019).
29. Locke, F. L., Zha, Y., Zheng, Y., Driessens, G. & Gajewski, T. F. Conditional deletion of PTEN in peripheral T cells augments TCR-mediated activation but does not abrogate CD28 dependency or prevent anergy induction. J Immunol 191, 1677 (2013).
30. Pauls, S. D. & Marshall, A. J. Regulation of immune cell signaling by SHIP1: A phosphatase, scaffold protein, and potential therapeutic target. Eur J Immunol 47, 932-945 (2017).
31. Kidani, Y. et al. Sterol regulatory element-binding proteins are essential for the metabolic programming of effector T cells and adaptive immunity. Nat Immunol 14, 489-499 (2013).
32. Yu, B. et al. Epigenetic landscapes reveal transcription factors that regulate CD8+ T cell differentiation. Nat Immunol 18, (2017).
33. Fernandez-Ruiz, D. et al. Liver-Resident Memory CD8+ T Cells Form a Front-Line Defense against Malaria Liver-Stage Infection. Immunity 45, 889-902 (2016).
34. Shimano, H. & Sato, R. SREBP-regulated lipid metabolism: convergent physiology divergent pathophysiology. Nat Rev Endocrinol 13, 710-730 (2017).
35. Zeiser, R. Immune modulatory effects of statins. Immunology 154, 69-75 (2018).
36. Sullivan, Z. A. et al. T6 T cells regulate the intestinal response to nutrient sensing. Science (1979) 371, (2021).
37. Menk, A. V. et al. 4-1BB costimulation induces T cell mitochondrial function and biogenesis enabling cancer immunotherapeutic responses. Journal of Experimental Medicine 215, (2018).
38. Scharping, N. E. et al. The Tumor Microenvironment Represses T Cell Mitochondrial Biogenesis to Drive Intratumoral T Cell Metabolic Insufficiency and Dysfunction. Immunity (2016) doi:10.1016/j.immuni.2016.07.009.
39. Zheng, L. et al. Pan-cancer single-cell landscape of tumor-infiltrating T cells. Science (1979) 374, (2021).
40. Lanterna, C. et al. The administration of drugs inhibiting cholesterol/oxysterol synthesis is safe and increases the efficacy of immunotherapeutic regimens in tumor-bearing mice. Cancer Immunol Immunother 65, 1303-1315 (2016).
41. Shires, J., Theodoridis, E. & Hayday, A. C. Biological Insights into TCRγδ+ and TCRαβ+Intraepithelial Lymphocytes Provided by Serial Analysis of Gene Expression (SAGE). Immunity 15, 419-434 (2001).
42. Fahrer, A. M. et al. Attributes of γδ intraepithelial lymphocytes as suggested by their transcriptional profile. Proc Natl Acad Sci USA 98, 10261-10266 (2001).
43. Hoytema van Konijnenburg, D. P. et al. Intestinal Epithelial and Intraepithelial T Cell Crosstalk Mediates a Dynamic Response to Infection. Cell 171, 783-794.e13 (2017).
44. Passi, S., Stancato, A., Aleo, E., Dmitrieva, A. & Littarru, G. P. Statins lower plasma and lymphocyte ubiquinol/ubiquinone without affecting other antioxidants and PUFA. BioFactors 18, 113-124 (2003).
45. Xu, S. et al. Uptake of oxidized lipids by the scavenger receptor CD36 promotes lipid peroxidation and dysfunction in CD8+ T cells in tumors. Immunity 54, 1561-1577.e7 (2021).
46. Ma, X. et al. Cholesterol Induces CD8+ T Cell Exhaustion in the Tumor Microenvironment. Cell Metab 30, 143-156.e5 (2019).
47. Mackay, L. K. et al. Hobit and Blimp1 instruct a universal transcriptional program of tissue residency in lymphocytes. Science 352, 459-463 (2016).
48. Kurd, N. S. et al. Early precursors and molecular determinants of tissue-resident memory CD8+ T lymphocytes revealed by single-cell RNA sequencing. Sci Immunol 5, (2020).
49. Boland, B. S. et al. Heterogeneity and clonal relationships of adaptive immune cells in ulcerative colitis revealed by single-cell analyses. Sci Immunol 5, (2020).
50. Milner, J. J. et al. Delineation of a molecularly distinct terminally differentiated memory CD8 T cell population. Proc Natl Acad Sci USA 117, 25667-25678 (2020).

### METHODS REFERENCES

1. O'Connor, A., et al. Responsiveness of cardiometabolic-related microbiota to diet is influenced by host genetics. Mamm Genome 25, 583-599 (2014).
2. Anderson, K. G. et al. Intravascular staining for discrimination of vascular and tissue leukocytes. Nature Protocols 2013 9:1 9, 209-222 (2014).
3. Steinert, E. M. et al. Quantifying Memory CD8 T Cells Reveals Regionalization of Immunosurveillance. Cell 161, 737-749 (2015).
4. McCausland, M. M. & Crotty, S. Quantitative PCR technique for detecting lymphocytic choriomeningitis virus in vivo. J Virol Methods 147, 167-176 (2008).
5. Crowl, J. et al. Tissue-resident memory CD8+ T cells possess unique transcriptional, epigenetic and functional adaptations to different tissue environments. Nat Immunol (2022).
6. Bankhead, P. et al. QuPath: Open source software for digital pathology image analysis. Scientific Reports 2017 7:1 7, 1-7 (2017).
7. Bolger, A. M., Lohse, M. & Usadel, B. Trimmomatic: a flexible trimmer for Illumina sequence data. Bioinformatics 30, 2114-2120 (2014).
8. Love, M. I., Huber, W. & Anders, S. Moderated estimation of fold change and dispersion for RNA-seq data with DESeq2. Genome Biol 15, 1-21 (2014).
9. Hanzelmann, S., Castelo, R. & Guinney, J. GSVA: Gene set variation analysis for microarray and RNA-Seq data. BMC Bioinformatics 14, 1-15 (2013).
10. Wei, J. et al. Targeting REGNASE-1 programs long-lived effector T cells for cancer therapy. Nature 576, 471-476 (2019).
11. Huang, H. et al. In vivo CRISPR screening reveals nutrient signaling processes underpinning CD8+ T cell fate decisions. Cell 184, 1245-1261.e21 (2021).
12. Li, W. et al. MAGeCK enables robust identification of essential genes from genome-scale CRISPR/Cas9 knockout screens. Genome Biol 15, 554 (2014).
13. Brinkman, E. K., Chen, T., Amendola, M. & Van Steensel, B. Easy quantitative assessment of genome editing by sequence trace decomposition. Nucleic Acids Res 42, e168-e168 (2014).
14. Korsunsky, I. et al. Fast, sensitive and accurate integration of single-cell data with Harmony. Nature Methods 2019 16:12 16, 1289-1296 (2019).
15. van Dijk, D. et al. Recovering Gene Interactions from Single-Cell Data Using Data Diffusion. Cell 174, 716-729.e27 (2018).
16. Gu, Z., Eils, R. et al. Complex heatmaps reveal patterns and correlations in multidimensional genomic data. Bioinformatics 32, 2847-2849 (2016).
17. Aran, D. et al. Reference-based analysis of lung single-cell sequencing reveals a transitional profibrotic macrophage. Nature Immunology 2019 20:2 20, 163-172 (2019).
18. Milner, J. J. et al. Runx3 programs CD8+ T cell residency in non-lymphoid tissues and tumours. Nature 552, 253-257 (2017).
19. Andreatta, M. et al. UCell: Robust and scalable single-cell gene signature scoring. Comput Struct Biotechnol J 19, 3796-3798 (2021).
20. Zheng, L. et al. Pan-cancer single-cell landscape of tumor-infiltrating T cells. Science (1979) 374, (2021).
21. Kushnareva, Y. et al. Functional Analysis of Immune Signature Genes in Th1* Memory Cells Links ISOC1 and Pyrimidine Metabolism to IFN-γ and IL-17 Production. J Immunol206, 1181-1193 (2021).
22. Ye, Q. et al. HORMA Domain Proteins and a Trip13-like ATPase Regulate Bacterial cGAS-like Enzymes to Mediate Bacteriophage Immunity. Mol Cell 77, 709-722.e7 (2020).
23. MacLean, B. et al. Skyline: an open source document editor for creating and analyzing targeted proteomics experiments. Bioinformatics 26, 966-968 (2010).

A number of embodiments of the invention have been described. Nevertheless, it can be understood that various modifications may be made without departing from the spirit and scope of the invention. Accordingly, other embodiments are within the scope of the following claims.

1: A CAR-T cell or a genetically engineered T cell or a pharmaceutical formulation comprising a CAR-T cell or a genetically engineered T cell,
wherein the CAR-T cell or the genetically engineered T cell is genetically modified to have no expression of, or substantially no expression or activity of, or less than wild type levels of expression or activity of, a squalene synthase or Fdft1 enzyme or gene.

2: The CAR-T cell or a genetically engineered T cell of claim 1, wherein the CAR-T cell or the genetically engineered T cell is formulated as a pharmaceutical, or formulated for in vivo administration.

3: A pharmaceutical formulation comprising a CAR-T cell or a genetically engineered T cell,
wherein the CAR-T cell or the genetically engineered T cell comprises or has contained therein a compound or composition capable of generating a phenotype of: no expression of, or substantially no expression or activity of, or less than wild type levels of expression or activity of, a squalene synthase or Fdft1 enzyme or gene.

4: The CAR-T cell or the genetically engineered T cell of claim 3, wherein the CAR-T cell or the genetically engineered T cell is formulated or manufactured as or with a pharmaceutical, an implant, a device, a patch, or is formulated for in vivo administration.

5: A product of manufacture or kit, or a pharmaceutical formulation, comprising a CAR-T cell or the genetically engineered T cell of claim 1.

6: A method for:
metabolically enhancing chimeric antigen receptor (CAR)-T cell function and persistence in vitro or in vivo, or
enhancing CAR-T cell efficacy in cancer immunotherapy,
the method comprising:
(a) administering to an individual in need thereof a CAR-T cell or a genetically engineered T cell with an inhibitor of a squalene synthase or Fdft1;
(b) administering to an individual in need thereof a CAR-T cell or a genetically engineered T cell that has been ex vivo exposed to an inhibitor of a squalene synthase or an Fdft1 inhibitor;
or
(c) administering to an individual in need thereof a CAR-T cell or a genetically engineered T cell that has been genetically modified to have a phenotype comprising: no expression of, or substantially no expression or activity of, or less than wild type levels of expression or activity of, a squalene synthase or Fdft1 enzyme or gene.

7: The method of claim 6, wherein the small molecule Fdft1 inhibitor comprises:
(a) mevastatin, or a compound having the formula: 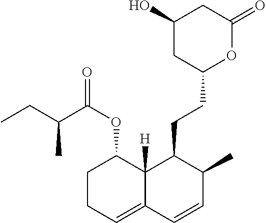 [SMILES: CC[C@H](C)C(=O)O[C@H]1CCC=C2C=C[C@H](C)[C@H](CC[C@@H]3C[C@@H](O)CC(=O)O3)[C@H]21]
(b) zaragozic acid A (ZAA), or a compound having the formula: 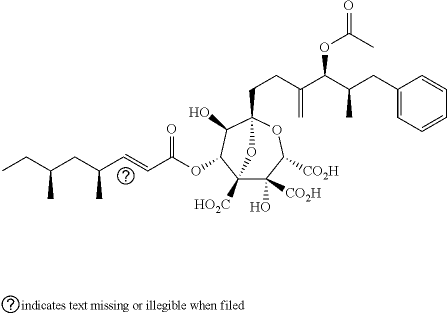
(c) a compound having the formula: 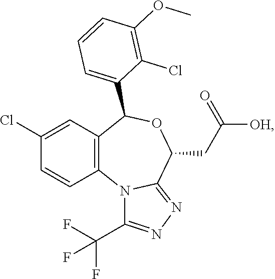 [SMILES: COc1cccc([C@H]2O[C@H](CC(=O)O)c3nnc(C(F)(F)F)n3-c3ccc(Cl)cc32)c1Cl]
(d) bavachinin, or a compound having the formula: 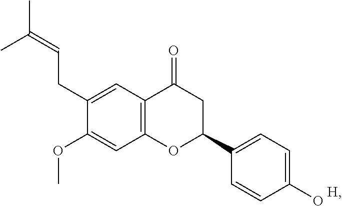 [SMILES: COc1cc2c(cc1CC=C(C)C)C(=O)C[C@@H](c1ccc(O)cc1)O2]
or
(e) a salt, an isomer, deuterated isoform, optical isomer or stereoisomer, a racemate or racemic mixture, an enantiomer, an individual diastereomer or a diastereomeric mixture, an analog, a crystalline product or a crystalline intermediate, a pharmaceutically acceptable salt thereof, or a prodrug or a bioisostere of any of (a) to (d).

8: The method of claim 7, wherein the inhibitor of the squalene synthase or Fdft1 inhibitor is formulated:
(a) in a liquid, a gel, a hydrogel, a vesicle, a liposome, a nanoparticle, a nanolipid particle, a powder or an aqueous or a saline formulation, or for administration in vitro or in vivo;
(b) for enteral or parenteral administration;
(c) in or as a liposome, a nanoparticle, or a nanoliposome;
(d) in or as a dendrimer, a tablet, a pill, a capsule, a gel, a hydrogel, a geltab, a liquid, a powder, an emulsion, a lotion, an aerosol, a spray, a lozenge, an aqueous or a sterile or an injectable solution, an eye drop, or an implant; or
(e) for intravenous injection, subcutaneous injection, intramuscular injection, inhalation, or intravitreal injection.

9-10. (canceled)

11: A method for treating or ameliorating a cancer in an individual in need thereof comprising administering to the individual in need thereof an inhibitor of a squalene synthase or an Fdft1 inhibitor,
wherein optionally the inhibitor of a squalene synthase or Fdft1 is a small molecule, a peptide or a polypeptide, or a nucleic acid inhibitor of squalene synthase or Fdft1 expression or activity,
and optionally the nucleic acid inhibitor of squalene synthase or Fdft1 inhibitor expression or activity comprises an antisense or miRNA molecule, or a CRISPR cassette or nucleic acid module,
and optionally the polypeptide inhibitor of squalene synthase or Fdft1 inhibitor expression or activity comprises an anti-squalene synthase or Fdft1 antibody.

12: The method of claim 11, further comprising administration, or co-administration, of a programmed cell death protein 1 (PD-1) inhibitor,
wherein optionally the PD1 inhibitor is pembrolizumab, nivolumab or cemiplimab.

13: The method of claim 11, wherein the small molecule Fdft1 inhibitor comprises:
(a) mevastatin, or a compound having the formula: 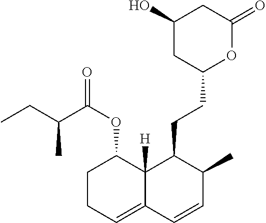 [SMILES: CC[C@H](C)C(=O)O[C@H]1CCC=C2C=C[C@H](C)[C@H](CC[C@@H]3C[C@@H](O)CC(=O)O3)[C@H]21]
(b) zaragozic acid A (ZAA), or a compound having the formula: 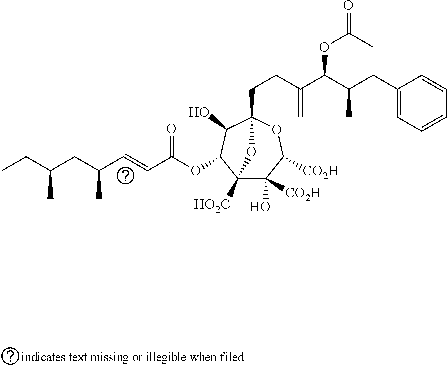
(c) a compound having the formula: 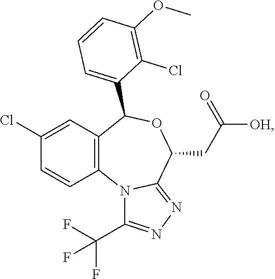 [SMILES: COc1cccc([C@H]2O[C@H](CC(=O)O)c3nnc(C(F)(F)F)n3-c3ccc(Cl)cc32)c1Cl]
(d) bavachinin, or a compound having the formula: 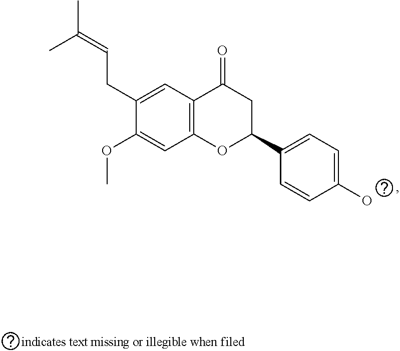 [SMILES: COc1cc2c(cc1CC=C(C)C)C(=O)C[C@@H](c1ccc(O)cc1)O2]
or
(e) a salt, an isomer, deuterated isoform, optical isomer or stereoisomer, a racemate or racemic mixture, an enantiomer, an individual diastereomer or a diastereomeric mixture, an analog, a crystalline product or a crystalline intermediate, a pharmaceutically acceptable salt thereof, a prodrug or a bioisostere of any of (a) to (d).

14: The method of claim 11, wherein the inhibitor of a squalene synthase or Fdft1 inhibitor is formulated:
(a) in a liquid, a gel, a hydrogel, a vesicle, a liposome, a nanoparticle, a nanolipid particle, a powder or an aqueous or a saline formulation, or for administration in vitro or in vivo;
(b) for enteral or parenteral administration;
(c) in or as a liposome, a nanoparticle, or a nanoliposome;
(d) in or as a dendrimer, a tablet, a pill, a capsule, a gel, a hydrogel, a geltab, a liquid, a powder, an emulsion, a lotion, an aerosol, a spray, a lozenge, an aqueous or a sterile or an injectable solution, an eye drop, or an implant; or
(e) for intravenous injection, subcutaneous injection, intramuscular injection, inhalation, or intravitreal injection.

15: An inhibitor of a squalene synthase or an Fdft1 inhibitor for use in treating cancer,
wherein optionally the inhibitor of a squalene synthase or Fdft1 is a small molecule, a peptide or a polypeptide, or a nucleic acid inhibitor of squalene synthase or Fdft1 expression or activity,
and optionally the nucleic acid inhibitor of squalene synthase or Fdft1 inhibitor expression or activity comprises an antisense or miRNA molecule, or a CRISPR cassette or nucleic acid module,
and optionally the polypeptide inhibitor of squalene synthase or Fdft1 inhibitor expression or activity comprises an anti-squalene synthase or Fdft1 antibody,
and optionally the inhibitor of a squalene synthase or the Fdft1 inhibitor is administered with, or formulated with, a programmed cell death protein 1 (PD-1) inhibitor.

16-17. (canceled)

18: The method of claim 1, wherein the genetically engineered T cell is a genetically engineered CD8⁺ T cell.

19: The method of claim 3, wherein the genetically engineered T cell is a genetically engineered CD8⁺ T cell.

20: The method of claim 6, wherein the inhibitor of a squalene synthase or Fdft1 inhibitor is a small molecule, a peptide or a polypeptide, or a nucleic acid inhibitor of squalene synthase or Fdft1 expression or activity.

21: The method of claim 6, wherein the CAR-T cell or the genetically engineered T cell is contacted with the squalene synthase or Fdft1 inhibitor before in vivo administration to an individual in need thereof, during or with in vivo administration of the CAR-T cell, and/or after in vivo administration of the CAR-T cell or the genetically engineered T cell.

22: The method of claim 6, wherein the inhibitor of a squalene synthase or Fdft1 inhibitor is a small molecule, a peptide or a polypeptide or a nucleic acid inhibitor of squalene synthase or Fdft1 expression or activity.

23: The method of claim 6, wherein the nucleic acid inhibitor of squalene synthase or Fdft1 inhibitor expression or activity comprises an antisense or miRNA molecule, or a CRISPR cassette or nucleic acid module.

24: The method of claim 6, wherein the polypeptide inhibitor of squalene synthase or Fdft1 inhibitor expression or activity comprises an anti-squalene synthase or Fdft1 antibody.
